SEQUENCE LISTING (182 sequences)

SEQ ID NO: 1 - DNA, 793 bp, Escherichia coli
catcaagctgtttgttcgtccgccggcggtgaaggggcgaccggatgatgtggccggcaaggtggagtggcagagggccggcaacaggctgaagggggttaacccgacgccgttttacatcaacctgtccacgctgacggtggggggtaaggaagtgaaggagcgtgaatatattgcgccgttttcctcccgtgaatatccgctgcctgcggggcatcgggtaaggttcagtggaaggtgataacggattacggcgggaccagtaagcagtttgaggcagagctgaagggttgaatacataaggtgataacagggtaaatgacgggctgacagatgcgtgatacttcttcagggcggatgagaacgggggtgacagggctggcgctggctgtgatggtggcctgtgtgatgtttcgtgcggagagtggtattgcgcgcacctactcctttgatgcggccatgctgaaaggtggcgggaagggggtggacctgaccctgtttgaggaaggtgggcagttacccggcatttatccggttgacattatcctgaatggttcccgtgtggattcacaggagatggcctttcacgcggagagggacgcggagggcaggccttatctgaagacctgtctgacccgtgagatgctggcgcgttacggggtcaggattgaggaatatccggcgttgttccgtgcatccggagagggtcgtggtgcctccgtggcggaggaggcctgtgctgacctgacggcgataccgcaggccacggagagttatcagtttgctgcccagc

SEQ ID NO: 2 - DNA, 470 bp, Escherichia coli
gcgatcatggccgcgaccagcactatcctcgcgatgagctcctcgcatgcagcgttcacaggaagtggtagcaccggtacgacaaaactaaccgttaccgaacagtgccaagtgctggtcaccggatctgacgtcaccaaaacgcgcggagaactcaccgacggggcccgtgtgggggtcctgtccgtaaccgcaaaaggctgtaacaccgagcatgcagcgttgcgtgcacagccagacaactaccaccagggcaagatcgtactgatccgcgatgactatcaggcacggataaatgtccgcttgcaggccaccgacgggcgtgcgtggaataccaacggcgacaccgtataccgcgccgatgctgggaactggggtggcagcttgttcgtagtcgtggacggggacaacgtggacaaaccgaccgggtcctacacactgaacctggactggggctactgggtgagttg

SEQ ID NO: 3 - DNA, 618 bp, Escherichia coli
gctaaatcaactgttgatgttgcaacggattctgttgatacatcctttatcatccgggacgactgtgcgatttctgttactgcgcagtctccaaaaacgtttaccttgagtgaagtcaaaaatcatgttagagcagcagatattaccattacacctacatgtggtggtaaatatttatgggcagaaatgaaagaggttgactctcagggatttggtattgcgagaactgatgctggtgatcttgcttctattacttgggtacaggatgggaattgggatgctggtgaaggtgagagagtggctaagacaaccctaccgaccgtggcttctcaaggggttccttatcctgcagtcttcgtaactcaggggagtacgggttaccgaagaaagctggagaatacaaatttggcctcactgttggctattgggtggagtgagttagaccgcaaaaattagtgttctctacaaagcatactgattgattaactttcaaggaagttcattatgcagggatgcagtcaagtgcaaaatcgtatcgtgattgttaccaacagtgtgaaagtgactcttctgttagcgccagtgatataagacggtaattcgccatttggattgtcc

SEQ ID NO: 4 - DNA, 351 bp, Escherichia coli
gttgaactgagtcttaataccagtgatggaaggagtggcgagttaaaagacggtacgaaggtggcaacaggaaggattatctgccgaggcacctatacaagttttcatatctggatgaatagcagacaaatgggaaatattcctggtcactatattatactgggtagacatgacagtcataatgaaatgcgggttaggctggatggcgcaggatggttgccatcggtaagtgatgggcaaggtatggtcagtaccgggatacctgagcagcatacatttgatgttgtgattgacggaaatcagctgcttgggcctgatgaatatatattatcagttagcggagaatgctca

SEQ ID NO: 5 - DNA, 432 bp, Escherichia coli
gcgttagaaagacctccaataaaagcaactgagacaatccgcctcaccgttacaaatgattgtcctgttactatagctacaaatagtccaccaaatgttggtgtatcgtcaacaacaccaataatatttaacgcaacagtaacgacgacagagcaatgtgctaaaagcggtgcaagggtctggttatggggaacaggtgccgctaataagtgggtcctagagcatactacaaatacaaaacaaaaatacacattaaatccatctatagatggaaattcatatttccagactccaggaactaatgcagcaatttataaaaatgtgacaaccagagacagagttctgaaggcaagtgtcaaggttgaccctaaaattcaagtattaataccaggcgaatatagaatgatactccatgccggaattaatttttaa

SEQ ID NO: 6 - DNA, 528 bp, Escherichia coli
tattaaaccatggtagcggggggatagacttaactctacttgagaaaggagggcagttgcctggtatttatccggttgatataattttaaatggttcgcgtattgattcaagggatatattcttttacacaaaaaaaaataggcatggtgaatattacctgaaaccctgtttaactcgagatattttgattaattacggagtaaaaacagaagaataccctaatcttttccggcaaaatagtgaaaaaaatagagacagtagcgattgtgctgacttatcagtgatcccccaagctacagaagactatcattttataaaacagcaactaatactcggaattccacaagttgcgatccgcccaccattgacaggcattgcccatgaaacaatgtgggatgatggtatatcagcatttttgttgaactggcaagtagaggggagtcattgggagtatagaagtaatactcgaaattcttcagacaatttttgggccagtttggaacctggaatcaatctcggatcttggc

SEQ ID NO: 7 - DNA, 586 bp, Escherichia coli
acagtatcatatggagccactccagacaggcctggattgtggcctcagagttagccagaggacatggttttgtccttgcaaaaaatacactgctggtattggcggttgtttccacaatcggaaatgcatttgcagtaaatatttcaggcacagtatcttcaggaggaactgtttcttccggcgaaacacaaatcgtgtattccggtcggggaaacagtaatgccactgtaaatagtggaggaacacaaatcgtcaataatggtgggaaaaccactgctacaactgttaatagttcaggaagccagaacgtcgggacttcaggagcaacaataagcacaattgtcaattctggtggcattcagcgagtcagttcaggtggtgtggcctctgcaacaaatttaagtggcggggctcagaacatctataatcttggccatgcatcaaataccgttatttttagcggtggaaatcagacgattttttcaggaggtataactgatagtacaaatatcagctccggtggccaacagcgtgtcagtagtggtggcgttgcctcgaacaccaccattaatagttctggcgcaca

SEQ ID NO: 8 - DNA, 324 bp, Escherichia coli
aatggtgcttgcgcttgctgccaccgttaccgcaggtgtgatgttttactaccagtctgcgtctgattccaataagtcgcagaatgctatttcagaagtaatgagcgcaacgtctgcaattaatggtctgtatattgggcagaccagttatagtggattggactcaacgattttacttaacacatctgcaattccggataattacaaagatacaacaaacaaaaaaataaccaacccatttgggggggaattaaatgtaggtccagcaaacaataacaccgcatttggttactatctgacgcttaccaggttggataaagcggc

SEQ ID NO: 9 - DNA, 505 bp, Escherichia coli
atggcgctaacttgccatgctgtgacagtaacagccactcatacagttgaatcagatgctgaattcacaatagattgggtcgacgctgggccaacgactacagatgcaaaagatggtgaggtttgggggcaccttgatatgactcaaaccaggggaacaccaacattcggaaaactccgcaatcctcaaggagagacttcgccaggaccgttgaaggcgccattcagttttaccgggccagatggtcatactgcaagagcgtaccttgattcatacggcgcaccgattcacaactacgcaggggataaccttgctaatggggtgaaggtaggtagtggaagcggaaacactccatttgttgttgggacagcaagtcgactaactgcaagaatcttcggagaccagacattggttccaggagtctaccggacaacctttgaattaactacttggaccgactgacggaaaattaacctgatgaaagaagggggctatatgtccccct

SEQ ID NO: 10 - DNA, 412 bp, Escherichia coli
caatagtcgcccacaggagttgtttatatatttctcacgtgttgatgcattcgctaacagagtaaatcttgcgattgtttcaaacagaagagctgatgaggtgattgtattacctcctccaactgttgtatcacgaccgatcatcggcattagaattggtaatgatgttttcttctcaacccatgcattggcgaatcggggcgtggattcaggagcaattgtaaatagtgtttttgagttcttcaacagacaaacggatcctataagacaggccgctaactggatgattgcaggagattttaaccgttcaccggctacactattttcaactcttgaaccagggattcgcaatcatgtaaatattattgctccaccagatccaacgcaagccagtggtggtgttcttgattat

SEQ ID NO: 11 - DNA, 556 bp, Escherichia coli
gaaagtaaatggaatataaatgtccggcaattaatttctggtgaaaatgctgtagacattttagctgtacaagaggcaggctctccgccgtcaacggctgtagatacaggtacacttattccttccccaggaattcccgtccgagagcttatctggaacttgtcgacaaatagcaggccacagcaagtatatatatatttttccgctgttgatgccctcggtggaagagtcaatcttgctctggttagcaatcggcgggccgatgaagtgtttgttcttagtcctgtaagacaaggtggacgaccattgcttggcatacgaattggtaatgatgcatttttcactgcacacgccatagctatgcgaaacaatgatgccccggctcttgttgaggaagtgtataacttcttccgcgacagcagagacccagtacaccaggcgcttaactggatgattcttggtgatttcaaccgtgaacctgcggatttagagatgaaccttactgttcccgtaagaagggcatcagaaattatttcaccagcggcggcaacacaaa

SEQ ID NO: 12 - DNA, 556 bp, Escherichia coli
gaaagtaaatggaatataaatgtccgacaattaatttctggtgaaaatgccgtagatattttagctgtgcaggaggcaggttctccgccatcaacggctgtagatacaggtagagttattccttccccaggcattcctgtccgggagcttatctggaacttgtctacaaatagcagaccacagcaagtatatatatatttttctgctgttgatgcctttggtggaagggtcaatcttgctctggttagcaatcggcaggccgatgaagtgtttgttcttcgcccggtaaggcaaggtgggcggccattgcttggcatacggattggcaatgatgcatttttcactgcacatgcgatagctacgcgaaacaatgacgctcccgctcttgttgaagaagtctatagcttctttcgtgacagccgagacccagtccaccaggccattaactggatgattcttggtgattttaatcgcgaacctgatgatttagaggtgaaccttacagttcctgtaagaaatgcatcagaaattattttccctgctgcaccgacacaaa

SEQ ID NO: 13 - DNA, 479 bp, Escherichia coli
ggtgcaatggctctgaccacaatgtttgtagcagtgagtgcttcagcagtagagaaaaatattactgtaacagctagtgttgatcctgcaattgatcttttgcaagctgatggcaatgctctgccatcagctgtaaagttagcttattctcccgcatcaaaaacttttgaaagttacagagtaatgactcaagttcatacaaacgatgcaactaaaaaagtaattgttaaacttgctgatacaccacagcttacagatgttctgaattcaactgttcaaatgcctatcagtgtgtcatggggaggacaagtattatctacaacagccaaagaatttgaagctgctgctttgggatattctgcatccggtgtaaatggcgtatcatcttctcaagagttagtaattagcgctgcacctaaaactgccggtaccgccccaactgcaggaaactattcaggagtagtatctcttgtaatgac

SEQ ID NO: 14 - DNA, 403 bp, Escherichia coli
gggcgctctctccttcaacaacactatcaaggaaatgacaggtgacagtaagctgctgaccatcactcagtctgaaccagctcctattcttttagggcgcacaaaagaggcgtttgcagcatcgattgttggtgttggtgcaattcctttaattgcgttcagtgattatgaagggaacggagttgccttacagagttctggggataacggtaaggggttctttgaattgcccatgaaagatgatagtggaaataatctcggtagcgtaaaagttaatgttacttctgctggcctgttttcctatagtgaaatatcaacaggtttagttggtataacttctgttgccagtggcgataatacaagtatttattatggtggtctggtgtcgccagcaattagggcg

SEQ ID NO: 15 - DNA, 1112 bp, Escherichia coli
gggggaagtacagaagaattacacgaaattttgttaggtcagggcccacagtcaagcttaggttttactgaatatacctcaaatgttaacagtgcagatgcagcaagcagacgacactttctggtagttataaaagtgcacgtaaaatatatcaccaataataatgtttcatatgttaatcattgggcaattcctgatgaagccccggttgaagtactggctgtggttgacaggagatttaattttcctgagccatcaacgcctcctgatatatcaaccatacgtaaattgttatctctacgatattttaaagaaagtatcgaaagcacctccaaatctaactttcagaaattaagtcgcggtaatattgatgtgcttaaaggacggggaagtatttcatcgacacgtcagcgtgcaatctatccgtattttgaagccgctaatgctgatgagcaacaacctctctttttctacatcaaaaaagatcgctttgataaccatggctatgatcagtatttctatgataatacagtggggctaaatggtattccaacattgaacacctatactggggaaattccatcagactcatcttcactcggctcaacttattggaagaagtataatcttactaatgaaacaagcataattcgtgtgtcaaattctgctcgtggggcgaatggtattaaaatagcacttgaggaagtccaggagggtaaaccagtaatcattacaagcggaaatctaagtggttgtacgacaattgttgcccgaaaagaaggatatatttataaggtacatactggtacaacaaaatctttggctggatttaccagtactaccggggtgaaaaaagcagttgaagtacttgagctacttacaaaagaaccaatacctcgcgtggagggaataatgagcaatgatttcttagtcgattatctgtcggaaaattttgaagattcattaataacttactcatcatctgaaaaaaaaccagatagtcaaatcactattattcgtgataatgtttctgttttcccttacttccttgataatatacctgaacatggctttggtacatcggcgactgtactggtgagagtggacggcaa

SEQ ID NO: 16 - DNA, 1241 bp, Escherichia coli
tatcatacggcaggaggaagcaccgaagaattgcatgagattttgttggggcaaggcccacagtcgagtttaggttttactgaatatacttcaaatattaacagtgcagatgcggcaagcagacgacattttcttgtagtcataaaagtgcaagtgaaatatataaacaataataacgtttcgcatgttaatcactgggcaattcctgatgaggctccagtagaagtactggctgtggttgacaggagatttaatttccctgagccatcaactccacctaatatatcaattatacacaagttgttatctctgagatattttaaagaaaatatcgaaagtacatcaaggcttaacttacagaaattaaatcgtggtaatattgatatatttaaagggagggggagtatttcatcaacacgtcagcgtgcgatttatccgtattttgaatctgctaatgctgatgagcaacaacctgtctttttctacatcaaaaaaaaccggtttgatgactttggctatgatcaatatttctataatagtacagtggggttgaatggtattcccacattgaacacctatactggagaaattctatcagacgcatcctcgctcggctcaacttattggaaaaagtataatctcactaatgaaacaagcatcattcgtgtatcaaattctgctcgaggggcaaatggtataaaaatagcacttgaagaagtgcaggaaggtaaaccggtaatcattacaagcggaaatttgagcggttgtacaacaattgttgctcgaaaaggaggatacctttataaggtacatacaggtacaacaatacctttagctggttttacaagtacaacaggggtaaaaaaagctgtagaagtttttgaattacttacaaataatccaatgccgcgcgtagagggagtaatgaataatgattttttggtaaattatctggcggaaagttttgatgagtctttaataacgtactcatcatctgaacaaaaaataggtagtaagattactatttctcgcgacaatgtttctacttttccttactttcttgataacataccagaaaaaggctttggtacatcggtgactatattggtaagagtagatggtaatgttatcgtaaaatccttatctgagagttattctttaaatgtagaaaactccaatatatcagtattgcatgttttttcaaaagatttttgattcggaaaattattgtctattgtgac

SEQ ID NO: 17 - DNA, 321 bp, Escherichia coli
gctcacaccatcaacaccgttgttcatacaaatgactcagataaaggtgttgttgtgaagctgtcagcagatccagtcctgtccaatgttctgaatccaaccctgcaaattcctgtttctgtgaatttcgcaggaaaaccactgagcacaacaggcattaccatcgactccaatgatctgaactttgcttcgagtggtgttaataaagtttcttctacgcagaaactttcaatccatgcagatgctactcgggtaactggcggcgcactaacagctggtcaatatcagggactcgtatcaattatcctgactaagtcaacg

SEQ ID NO: 18 - DNA, 401 bp, Escherichia coli
gggcccactctaaccaaagaactggcattaaatgtgctttctcctgcagctctggatgcaacttgggctcctcaggataatttaacattatccaatactggcgtttctaatactttggtgggtgttttgactctttcaaataccagtattgatacagttagcattgcgagtacaagtgtttctgatacatctaagaatggtacagtaacttttgcacatgagacaaataactctgctagctttgccaccaccatttcaacagataatgccaacattacgttggataaaaatgctggaaatacgattgttaaaactacaaatgggagtcagttgccaactaatttaccacttaagtttattaccactgaaggtaacgaacatttagtttcaggtaattaccg

SEQ ID NO: 19 - DNA, 731 bp, Escherichia coli
gcacaattactgctgatgcgtataaagacaaatgggaatggatggttgggggcgctctctccttcaacaacactatcaaggaaatgacaggtgacagtaagctgctgaccatcactcagtctgaaccagctcctattcttttagggcgcacaaaagaggcgtttgcagcatcgattgttggtgttggtgcaattcctttaattgcgttcagtgattatgaagggaaaggagttgccttacagagttctggggataacggtaaggggttctttgaattgcccatgaaagatgatagtggaaataatctcggtagcgtaaaagttaatgttacttctgctggcctgttttcctatagtgaaatatcaacaggtttagttggtataacttctgttgccagtggcgataatacaagtatttattatggtggtctggtgtcgccagcaattagggcgggtaaagacgcagcatcagctgtgtcgaaatttggcaactataatcatacacaattgctgggccagcttcaagcagtaaaccctaacgcgggcaatagaggacaagtaaataaaaatagtgcggtctcacaaaatatggtgatgactactggtgatgtaattgcatcctcttacgcacttggtattgaccagggacagactattgaagcaacctttactaatcctgtggttagcaccacccagtggagtgctccgctgaacgtggcagtaacttataac

SEQ ID NO: 20 - DNA, 677 bp, Escherichia coli
cacacacaaacgggagctgtttgtagcgaagccactcgttcaaatcaattctcttgacgtggggaaatccgttttccaagcggaccccttatagggggttgagggcctcctacccttcactcttgactatgttaacgataatcattatcgttagtgtttgtgtggtaatgggatagaaagtaatgggataaaaagtaatggatagaaaaagaacaaaattagagttgttatttgcatttataataaatgccaccgcaatatatattgcattagctatatatgattgtgtttttagaggaaaggactttttatccatgcatacattttgcttctctgcattaatgtctgcaatatgttactttgttggtgataattattattcaatatccgataagataaaaaggagatcatatgagaactctgactctaaatgaattagattctgtttctggtggtgcttcagggcgtgatattgcgatggctataggaacactatccgggcaatttgttgcaggaggaattggagcagctgctgggggtgtggctggaggtgcaatatatgactatgcatccactcacaaacctaatcctgcaatgtctccatccggtttagggggaacaattaagcaaaaacccgaagggataccttcagaagcatggaactatgctgcgggaag

SEQ ID NO: 21 - DNA, 260 bp, Escherichia coli
cgtgtgggagccctgagccttaacgcgagagggtgtaacaccgagcatgcagcgctgcgcgctcaagcagacaactaccacaatggcaagatcgtactgctacgtgaagaccaacaagcgcggataaatgtgcgcttggtggcatccgatgggggccaatggactaacgacggcgcaaccacataccgcgacgctgccggggactggggcgggagcttgtacgtagtcgtggacggggacaatactagcaaccaggccgg

SEQ ID NO: 22 - DNA, 791 bp, Escherichia coli
cattatggaacggcagaggttaatctgcagagtggtaataactttgacggtagttcactggacttcttattaccgttctatgattccgaaaaaatgctggcatttggtcaggtcggagcgcgttacattgactcccgctttacggcaaatttaggtgcgggtcagcgttttttccttcctgaaaatatgttgggctataacgtcttcattgatcaggatttttctggtgataatacccgtttaggtattggtggcgaatactggcgagactatttcaaaagtagtattaacggctatttccgcatgagcggctggcatgagtcatacaataagaaagactatgatgagcgcccagcaaatggcttcgatatccgttttaatggctatctgccatcatacccggcattaggtgccaagctgatgtatgagcagtattatggtgataatgttgctttgtttaattctgataagctgcagtcgaatcctggtgcggcgaccgttggtgtaaactatactccgattcctctggtgacgatggggatcgattaccgtcatggtacgggtaatgaaaatgatctcctttactcaatgcagttccgttatcagtttgataaaccgtggtctcagcaaattgagccacaatatgttaacgagttaagaacattatcaggcagccgttacgatctggttcagcgtaataacaatattattctggagtacaaaaagcaggatattctttctctgaatattccgcatgatattaatggtactgaacgcagtacgcagaagat

SEQ ID NO: 23 - DNA, 397 bp, Escherichia coli
  misc_feature (224)..(224): n is a, c, g, or t
cagggtaaaagaaagatgataagttaacgcttggagtgatcgaacgggatccaaatcactgatcgatgttccatgcgaataagtgatcgatcatgtcggaatatccaaaaacccgaaatcaccagttgccacattgaacggcgctggtgatttcgggttcgtcactttatggataccatcaacccattccccggagaaagtatgggctggctaaagtgtagcgncattaagagcagttatttagtattttaatgagtatcgaatctttatatttgcatcattccgttgttggtccgccttctgacaagctgtgttggcagaagaaacgtcgttagcggttcctattttgttactacctagatatatatcaggtttttgataatacatggtccccata

SEQ ID NO: 24 - DNA, 117 bp, Escherichia coli
tcggatgccatcaacacagtatatccgaaggcccgcatccagttatgcatcgtgcatatggtgcacaacagcctgcgcttcgtgtcatggaaggactacaaagccgtcactcgcgac

SEQ ID NO: 25 - DNA, 159 bp, Escherichia coli
gtttattctggggcaggctcatcagaagtatttgctggtgaaggtcatgataccgtatcttataataagacggatgttggtaaactaacaattgatgcaacaggagcatcaaaacctggtgaatatatagtttcaaaaaatatgtatggtgacgtgaag

SEQ ID NO: 26 - DNA, 525 bp, Escherichia coli
ggatacatcaactgcaacatcagttgctagtgcgaacgcgagtacttcgacatcgacagtctatgacttaggcagtatgtcgaaagacgaagtagttcagctatttaataaagtcggtgttttcagtgcgcttctcatgtttgcctatatgtatcaggcacaaagcgatctgtcgattgcaaagtttgctgatatgaatgaggcatctaaggagtcaaccacagcccaaaaaatggctaatcttgtggatgctaaaattgctgatgttcagagtagttctgacaagaatgcaaaagccaaactacctaaagaagtgattgactatataaatgatcctcgcaatgacattacagtaagtggtattagcgatctaaatgctgaattaggcgctggtgatttgcaaacggtgaaggccgctatttcggccaaatcgaataacttgaccacggtagtgaataatagccagcttgaaatacagcaaatgtcaaatacgttaaacctattaacgagtgcacgttctgatat

SEQ ID NO: 27 - DNA, 523 bp, Escherichia coli
cgacatcgacgatctatgacttaggtaatatgtcgaaggatgaggtggttaagctatttgaggaactcggtgtttttcaggctgcgattctcatgttttcttatatgtatcaggcacaaagtaatctgtcgattgcaaagtttgctgatatgaatgaggcatctaaagcgtcaaccacggcacaaaagatggctaatcttgtggatgccaaaattgctgatgttcagagtagcactgataagaatgcgaaagccaaacttcctcaagacgtgattgactatataaacgatccacgtaatgacataagtgtaactggtattcgtgatcttagtggtgatttaagcgctggtgatctgcaaacagtgaaggcggctatttcagctaaagcgaataacctgacaacggtagtgaataatagccagctcgaaattcagcaaatgtcgaatacattaaatctcttaacgagtgcacgttctgatgtgcaatctctacaatatagaactatttcagcaatatcccttgg

SEQ ID NO: 28 - DNA, 487 bp, Escherichia coli
gggaacatgtcgaaagatgaagttgttgagctgtttaaaaaagttggcgtatttcaggctgcgcttatcatgtttgcttatatgtaccaggcacaaagcgagctatctattgctacatatgcagacatgaatgagtcatctaaggaatccaccgaggcacaaaaaatggccaatttggtggatgccaagatcgctgaagttcagtctagttcggaaaaggataaaaaggtcaaacttcctgacgaagtaattagttatattcaagattcacgaaatgggatttccgtaagtagtgatattgacatcaccaaagagttgggtgctggtgacctgcaaaccgtaaaagctgctatttcagcaaaagcaaataacctgacaacgacggtgaataataaccagcttacgttgcagcaaatgtctaatacgctgaatttattaacaaatgctcgttcagatatgcagtcgttgcaatatcgaactattcagg

SEQ ID NO: 29 - DNA, 502 bp, Escherichia coli
cggagagtacgaccggcgcttccagtgcagttgccgcatctgctttatcaattgattcatctctgcttactgatggtaaggttgatatttgtaagctgatgctggaaattcaaaaactcctcggcaagatggtgactctattgcaggattaccaacaaaaacaattggcgcaaagctatcagattcagcaggccgtttttgagagccagaataaagctattgaggaaaaaaaagccgcggcaaccgctgctttggttggcgggattatttcatcagcattggggatcttaggttcttttgcagcaatgaacaacgcggctaaaggggctggtgagattgctgaaaaagcaagctctgcatcttcaaaggctgctggtgcggcttctgaggttgcaaataaagctctggtcaaggctacggaaagtgttgctgatgtcgcagaggaggcatccagtgcgatgcagaaagcgatggccacaacaacgaaagcagccagccgtgc

SEQ ID NO: 30 - DNA, 377 bp, Escherichia coli
gctgccattaatagcgcaactaaaggcgcgagtgatgtcgctcagcaagccgcttctacttctgcgaagtctatcggtacagtctctgaagcttcaactaaagcactggcgaaggcttccgaaggtattgcagatgcagcagatgatgcagctggcgcaatgcagcaaactatcgcgacagctgcaaaagcggccagtcgtacatccggtatcactgatgatgttgctacttcggctcagaaagcttctcaggtagctgaagaggctgctgatgctgctcaagaattagcacagaaggcaggattattaagtcgctttactgctgctgccggaaggatttccggttcaacgccatttattgttgttaccagccttgc

SEQ ID NO: 31 - DNA, 395 bp, Escherichia coli
gtaatgacggttaattctgtttcggagaatactaccggctctaatgcaattaccgcatctgctattaattcatctttgcttaccgatggtaaggtcgatgtttctaaactgatgctggaaattcaaaaactcctgggcaagatggtgcgtatattgcaggattaccaacagcaacagttgtcgcagagctatcagatccaactggccgtttttgagagccagaataaagccattgatgaaaaaaaggccgctgcaacagccgctctggttggtggggctatttcatcagtattggggatcttaggctcttttgcagcaattaacagtgctacgaaaggcgcgagtgatattgctcaaaaaaccgcctctacatcttctaaggctattgatgcggc

SEQ ID NO: 32 - DNA, 500 bp, Escherichia coli
cccataacggaacaactcatcatgcaataagcacccaaaactggggacaaagctcatataaatatatagaccggatgacgaatggagattttgctgtaacacgacttgataagtttgttgttgaaacaacaggggtaaaaaattcagtagatttttctctcaatagtcatgatgctcttgaacgttatggtgtggagatcaatggtgagaaaaaaatcattggtttcagggttggggctgggacgacttataccgttcaaaatggtaatacatatagtacaggacaggtatacaatcctcttttgttaagcgcttcaatgtttcagttaaactgggataacaaaagaccatataataacacgacacctttttataatgaaactaccggtggagacagtggttccggtttctatctgtatgataacgtaaaaaaagaatgggttatgcttggtactttatttggaatagcatccagtggtgcagatgtttggtctattctg

SEQ ID NO: 33 - DNA, 1830 bp, Escherichia coli
aaacagcaggcacttgaacgttacggggttaattataaaggagaaaagaaacttatcgcattcagagccggctctggtgtggtatccgttaaaaaaaatggacgcataactccatttaatgaggtttcttataagccagaaatgttaaatggctctttcgttcacattgatgactggagtggatggctgatattaaccaacaaccagtttgatgagtttaataacattgcctctcagggtgacagcggttcagcactgttcgtctatgataaccaaaagaaaaagtgggttgtcgctggaactgtctgggggatttataattacgccaatggcaaaaaccacgcagcatacagtaaatggaaccagacaaccattgacaacctgaagaacaagtattcttacaacgtggatatgtcaggggctcaggttgcaaccattgaaaatggaaaactgacaggcactggctcagacaccaccgatataaaaaataaggacttaatatttactggcggtggagatatcctcctgaaatcctcttttgataatggtgctggcggtcttgtctttaatgataaaaagacctatcgagtaaacggggatgatttcacctttaaaggtgccggtgttgatacaagaaacggcagcaccgttgagtggaatatccggtatgataataaagacaaccttcacaaaattggtgatggcacattagatgtccgaaaaacccagaacaccaacctgaaaacaggtgagggtcttgtcattcttggagctgaaaaaacattcaataatatctacataaccagtggtgatggaactgtccgactgaatgcagaaaatgcactgtctggcggtgaatacaacggtattttctttgcgaaaaatggcggaactcttgacctgaacggatataatcagtctttcaataaaattgctgcaactgattcaggtgctgtaataaccaatacgtcaaccaaaaaatccattttatccctgaataatactgctgactatatctatcacggtaacataaacgggaatctggacgtacttcagcatcatgagacgaaaaaagagaaccgtcgtcttattcttgatgggggcgtggacacaacaaatgatataagcctgcgtaatacacaactgtccatgcagggacatgccactgaacatgccatttatcgggatggagctttctcttgttcactaccagctcctatgcgctttttgtgtggcagtgattatgttgcaggaatgcaaaatacagaagctgatgctgtaaaacaaaacggaaatgcctataaaaccaacaatgctgtctctgatttatcgcagccagactgggaaaccggaacattcagatttggaacgctacatcttgaaaattccgatttttctgttggtcgtaatgcaaatgtaatcggggacattcaggccagtaaatcaaacattactattggtgacactacagcatatattgatttgcatgctggtaaaaatattaccggtgatggttttggcttccgccagaatattgtgcgtggaaactcacaaggagaaacgctgtttacaggagggatcacagcagaagacagcactatcgttattaaagataaagcaaaagcattattttcaaattatgtatacctgctgaacacaaaagcaaccatagagaacggtgctgatgtgacaactcaaagtggtatgttctccacgagcgatatcagcatctctggtaatctgtccatgacaggcaatcccgacaaagacaataaattcgagccctcaatatatctgaatgatgcttcttatctactgactgacgactcc

SEQ ID NO: 34 - DNA, 499 bp, Escherichia coli
ggccactttcaatgttggtcaggaggtcccggtactttcgggctcacagacaacctctggggacaatatttttaacacggtcgagcgcaaaacggtggggatcaaactcagggtaaaaccccagatcaacgagggtgattccgtgttactggagatagaacaggaggtgtccggtgtggcggacactgcagtagccaccactactgacttgggagcaaccttcaacacccgaacagtgaccaatgccatgctggtcgggaatggcgaaacggtggtggtcggaggattactggataagtcgatcagggggagtgagagtaaagtgccactgctgggggatatcccggtactggggcatctttttcgcgcaaaaagcgaacagacagctaagcgtaatctgatgctgttcattcggccaactattattcgtgagcgcgacggatttcgtcatgcttcggccgaaaaataccagtcgtttaatcaggaacaggtgcagtcg

SEQ ID NO: 35 - DNA, 441 bp, Escherichia coli
atgcagaaaattcaatttatccttggaatactggcggctgcatcatcttctgctacgcttgcttatgacggtaaaattacttttaatggaaaagttgttgatcaaacttgttctgttacaacagaaagcaagaatttgacagttaagttaccaactgtctctgctaattcattagcttcaagcggaaaagtggtgggacttactcctttcacaattttgctggaagggtgcaatacgcctgccgtgacaggtgctcagaatgtaaatgcttatttcgaacctaatgcgaacacggattacaccactggtaatttaactaatacggcttcttctggtgcatctaatgttcagattcagctactgaatgcagatggggttaaagctattaaacttggtcaggctgctgcagctcagagtgtggatacagttgctattaatgat

SEQ ID NO: 36 - DNA, 950 bp, Escherichia coli
tgttggaccgtctcagggctcttattccagcactcatgcaatggataacctgccatttgtctataataccggttacaacattggatatcagaatgcaaatgtctggcgtattagtggcgggttttgtgttggtctggacgggaaagtggatttacccgtggttggcagtcttgacgggcagagtatttatgggctgacggaggaggtgggactccttatatggatgggggacacgaattattccaggggtaccgcgatgagtggaaactcatgggagaatgtcttttccggatggtgcgtgggaaattatgtatcaacgcagggactgtctgttcacgtaagaccggtaattttaaaaagaaattcctctgcgcaatacagtgtacagaaaaccagtatcgggagtatcagaatgaggccctataacggttcatctgcaggcagtgttcagaccacagtgaatttcagcctgaatccatttacgctgaatgacacagtaacatcgtgcagattactgacaccttccgcagtcaatgtcagcctggctgcaatttctgccggacaactaccatcatccggtgatgaagttgtcgccgggacaacatcactgaaattacagtgtgatgccggagtaacagtatgggcaacactgactgatgcgaccacaccgtccaacagaagcgatatactcacactgacgggggcatcgactgcaaccggagtcgggctgagaatatacaaaaacactgacagtacgcccctgaagtttggacctgattcgccggtaaagggaaatgaaaaccagtggcagttatcgacaggaacggaaacgtcaccctcagtccggttgtatgtaaagtatgtgaatactggtgagggaattaatccgggtacggttaacggaatatcaacatttacgttttcctatcagtaacagcgagttccgggag

SEQ ID NO: 37 - DNA, 510 bp, Escherichia coli
gtgaaaagactagtgtttatttcttttgttgcgctgtccatgacagcgggttccgcaatggctcagcaaggggatgttaaattctttggtaacgtatcagcaactacctgtaatttgacaccacaaataagtggcactgtaggagataccattcagcttggtactgttgcaccaagcggaactggtagtgaaattccttttgcactgaaggcttcttcaaatgttggcggttgtgcttccttgtccactaaaacagctgatataacttggagcgggcagttaaccgaaaaaggttttgctaatcaagggggggtggcaaatgattcatatgtcgctctgaaaaccgtgaacggtaaaacacaggggcaggaggttaaggcgtcgaatagcactgtaagtttcgatgcatcaaaagcaactacggaaggtttcaaatttactgctcaactgaaaggtggtcaaaccccgggtgacttccagggggcagcggcttacgcggttacttacaag

SEQ ID NO: 38 - DNA, 858 bp, Escherichia coli
atgaaaaagactctgattgcactggcaattgctgcatctgctgcatctggtatggcacatgcctggatgactggtgatttcaatggttcggtcgatatcggtggtagtatcactgcagatgattatcgtcagaaatgggaatggaaagttggtacaggtcttaatggatttggtaatgtattgaatgacctgaccaatggtggaaccaaactgaccattactgttactggtaataagccaattttgttaggccgaaccaaagaagcatttgctacgccagtaagtggtggtgtagatggaattcctcagattgcatttactgactatgaaggagcttctgtaaaactcagaaacactgatggtgaaactaataaaggtttagcatattttgttctgccgatgaaaaatgcagagggcactaaagttggttcagtgaaagtgaatgcatcttatgccggtgtgttcgggaaaggtggggttacttctgcggacggggagctgttttcgctttttgccgacgggttgcgcgctatcttttatggtggtttgacgacgactgtttcgggtgctgcactcacgagtgggagtgccgcagcggcgcgcacagagttgtttggaagtctatcaagaaatgatattctcggacagattcaaagagtaaacgcaaatattacttctcttgttgacgtcgcaggttcttacagggaagacatggagtacactgatggaactgttgtttctgctgcctatgcactgggtattgcaaacggtcagactattgaggcaacttttaatcaggctgtaactaccagcactcagtggagcgctccgctgaacgtagcaattacttattactaa

SEQ ID NO: 39 - DNA, 431 bp, Escherichia coli
gagggactttcatcttttagcaatactacaaatgaaattgttaaacggaagttgaatatttctgttccaacggatgaattatttttagcagcgaagatgagtgatgggattaaaggtgttttcgtagggaatacactcattcctaagattgaaatggcatcttatgatggtagtgttattacacctagtttcacttcaaatacagcaatggatattgctgtaaaagtaaaaaactcaggtgataatactgagctagggactctttctgttcctttgtcatttggtgcggcagttgcaactatttttgatggcgatactactgatagcgctgtagcgcatattatcggtggttctgctggtacagtatttgaagggcttgttaatccaggtcgatttactgatcagaatatagcctataaatggaatggact

SEQ ID NO: 40 - DNA, 450 bp, Escherichia coli
tgcgactaccaatgcttctgcgaatacaggtactattaacttcaatggcaaaataacgagtgctacttgtacaattgaccctgaggtcaatggtaatcgtacatcaactatagatcttgggcaggctgctattagtggtcatggcactgtagtggattttaaactaaaaccagcgcccggcagtaatgactgcctagcgaaaacaaatgctcgtattgactggtctggttctatgaacagtttaggttttaataatacagcttcaggaaatactgctgctaaaggataccatatgactttgcgcgcaacaaacgttggaaatgggtctggtggtgctaatattaatacttcattcactacggctgaatacactcacacttctgcaattcagtcatttaactattcagcccagctgaaaaaagatgaccgcgctccgtctaatggtggata

SEQ ID NO: 41 - DNA, 954 bp, Escherichia coli
aaatttagaaaagtgcattatgcttatcactagataagaaaataaaacacgaaatatagcgagccatatagcctgttgtgtttgtaatagataaaaaacacgcaattgattatttatgtatctttttgtttgtatttttttattaaaaaaagcacacaattactgcgtgcatcgaaatgagttgaagtggatgcatatatgcatgaaatgcttttaacttgaaagtcttaatgtttctattaattaagataaggtaatatgagaatgaaaaaatccgcattaacattagcagtgctttcctctctgttcagtggttactcgctcgcagcgcccgctgaaaacaacaccagccaggcaaatttagactttactggtaaagttactgccagtctatgccaagtggatacttctaatctgtcgcaaaccatagatcttggagagttgtctacttctgctcttaaagctactggcaaggggcctgccaagtcatttgcagttaatcttatcaactgcgatacaacattgaattctattaaatacactattgctggtaataataatacaggaagtgatactaaatatttagttccagcctccaatgatactagtgcatcaggagttggcgtatacattcaggacaacaacgcccaggctgtggaaattggtactgaaaaaactgtacctgtggtatcaaatggcggattagctctttcagaccaaagtattccactgcaagcatacatcggaaccaccacagggaatcctgatacaaacggtggagttacggccggtactgtcactgctagtgcagtaatgactattcgttcagcaggtacaccgtaattagataacaatttttatacaacaaaacaggaaggattttgaactaatccttcctgttattggagattgaaatgtctaagtttgtaatatttcttgtgtttttgtttatat

SEQ ID NO: 42 - DNA, 331 bp, Escherichia coli
gttgatcaaaccgttcagttaggacaggttcgtaccgccactttgaagcaggctggagcaaccagctctgctgtcggttttaacattcagctgaatgattgcgataccactgttgccacaaaagccgctgttgccttcttggggacggcgattgacagtactcatcctaaagtcctggctctacagagttcagctgcgggtagcgcaacaaacgttggcgtgcagattctggacagaacaggtaatgagctgacgctggacggtgcgacatttagtgcagaaacaaccctgaataacggtactaacaccattccgttccaggcgcgttatt

SEQ ID NO: 43 - DNA, 506 bp, Escherichia coli
tcgagaacggataagccgtggccggtggcgctttatttgacgcctgtgagcagtgcgggcggggtggcgattaaagctggctcattaattgccgtgcttattttgcgacagaccaacaactataacagcgatgatttccagtttgtgtagaatatttacgccaataatgatgtggtggtgcctactggcggctgcgatgtttctgctcgtgatgtcaccgttactctgccggactaccctggttcagtgccaattcctcttaccgtttattgtgcgaaaagccaaaacctggggtattacctctccggcacaaccgcagatgcgggcaactcgattttcaccaataccgcgtcgttttcacctgcacagggcgtcggcgtacagttgacgcgcaacggtacgattattccagcgaataacacggtatcgttaggagcagtagggacttcggcggtgagtctgggattaacggcaaattatgcacgtaccggagggcaggtgactgc

SEQ ID NO: 44 - DNA, 625 bp, Escherichia coli
gcgctgtcgagttctatcgagcgtctgtcttctggcttgcgtattaacagcgcgaaggatgacgccgcaggtcaggcgattgctaaccgttttacttctaacattaaaggcctgactcaggcggcccgtaacgccaacgacggtatttctgttgcgcagaccaccgaaggcgcgctgtccgaaatcaacaacaacttacagcgtattcgtgaactgacggttcaggccactacagggactaactccgattctgacctggactccatccaggacgaaatcaaatctcgtcttgatgaaattgaccgcgtatccggccagacccagttcaacggcgtgaacgtgctggcgaaagacggttcaatgaaaattcaggttggtgcgaatgacggcgaaaccatcacgatcgacctgaaaaaaatcgattctgatactctgggtctgaatggctttaacgtaaatggtaaaggtactattaccaacaaagctgcaacggtaagtgatttaacttctgctggcgcgaagttaaacaccacgacaggtctttatgatctgaaaaccgaaaataccttgttaactaccgatgctgcattcgataaattagggaatggcgataaagtcacagttg

SEQ ID NO: 45 - DNA, 359 bp, Escherichia coli
cagcacaggcagtggatacgacgattactgttacagggagggtattgccacgtacctgtaccattggtaatggaggaaacccaaacgccaccgttgttttggataacgcttacacttctgacctgatagcagccaacagcacctctcagtggaaaaatttttcgttgacattgacgaattgtcagaatgtaaacaatgttactagctttggtggaaccgcagaaaatacaaattattacagaaatacaggggatgctactaatatcatggttgagctacaggaacaaggtaatggtaatacccccttgaaagttggttcaacaaaagttgttacagtgagcaatgggcaggcgacattc

SEQ ID NO: 46 - DNA, 207 bp, Escherichia coli
ggcggcgtgcgcttctcgcatgataaatccagtacacaatatcacggcagcatgctcggcaacccgtttggcgaccagggtaagagcaatgacgatcaggtgctcgggcagctatccgcaggctatatgctgaccgatgactggagagtgtatacccgtgtagcccagggatataaaccttccgggtacaacatcgtgcctactgcg

SEQ ID NO: 47 - DNA, 500 bp, Escherichia coli
tgttgaaagatcagtcctcattacccagcaacattgggatacgctgataggtgagttagctggtgtcaccagaaatggagacaaaacactcagtggtaaaagttatattgactattatgaagaaggaaaacgtctggagaaaaaaccggatgaattccagaagcaagtctttgacccattgaaaggaaatattgacctttctgacagcaaatcttctacgttattgaaatttgttacgccattgttaactcccggtgaggaaattcgtgaaaggaggcagtccggaaaatatgaatatattaccgagttattagtcaagggtgttgataaatggacggtgaagggggttcaggacaaggggtctgtatatgattactctaacctgattcagcatgcatcagtcggtaataaccagtatcgggaaattcgtattgagtcacacctgggagacggggatgataaggtctttttatctgccggctcagccaatatctacgcag

SEQ ID NO: 48 - DNA, 556 bp, Escherichia coli
aggttcttgggcatgtatcctggctctgggccagttccccattacacagaaactggccagtctctttgtttgcaataaatgtattacctgcaatacgggctaaccaatatgctttattaacccgggataattaccctgttgcatattgtagttgggctaatttaagtttagaaaatgaaattaaatatcttaatgatgttacttcattagtcgcagaagactggacttctggtgatcgtaaatggttcattgtctggattgctcctttcggggataacggtgccctgtacaaatatatgcgaaaaaaattccctgatgaactattcagagccatcagggtggatcccaaaactcatgttggtaaagtatcagaatttcacggaggtaaaattgataaacagttagcgaataaaatttttaaacaatatcaccacgagttaataactgaagtaaaaaacaagtcagatttcaatttttcattaacaggttaagaggtaattaaatgccaacaataaccgctgcacaaattaaaagcacactgcagtctgcaaagcaa

SEQ ID NO: 49 - DNA, 170 bp, Escherichia coli
aggcaggtgtgcgccgcgtactacacattaccgccgttgatgttatcaagcagggcaataatttactcggcgtaataacagagagtaaatctggtcgtcaggctattttggcaaatgtcattattgactgtactggtgatgctgatattgcatggtttgccggagcacca

SEQ ID NO: 50 - DNA, 827 bp, Escherichia coli
ctggcggaggctctgagatcagtagagggtgtggatgttgaaagtggtacgggtaaaaccggagggctggaaatcagcatccgaggaatgccagccagttacacgctgatactgattgatggtgttcgtcagggcggaagcagtgacgtgactcccaacggtttttctgccatgaataccgggttcatgccccctctggccgccattgagcgtattgaggttatcagggggccgatgtccacactgtatggctctgatgcgatgggcggtgtggtgaatatcattaccagaaagaatgcagacaaatggctctcttccgtcaatgcagggctgaatctgcaggaaagcaacaaatggggtaacagcagccagtttaatttctggagcagtggtccccttgtggatgattctgtcagcctgcaggtacgcggtagcacacaacagcgtcagggttcatcggtcacatcactgagcgatacagcaggcacgcgtattccttatcccacggagtcacagaattataatcttggtgcacgtcttgactggaaggcgtcggagcaggatgtgctctggtttgatatggataccacccggcagcgttatgataaccgggatgggcaactggggagtctgacggggggatatgaccggaccctgcgctatgagcgaaacaaaatttcagctggctatgatcatactttcaccttcggaacatggaaatcgtatctgaactggaacgagacagaaaataaaggtcgtgagcttgtacgcagtgtactgaagcgcgacaaatgggggcttgccggtcagccgcgggagcttaagga

SEQ ID NO: 51 - DNA, 258 bp, Escherichia coli
tctgatatagtttatatgggtaataaggctctttatttaatccttatcttttccttatggccagtaggtatagctacggttattggattaactattggtttattacagacagtgactcaacttcaagagcagacacttccttttggtataaagcttataggtgtctcaatatctttgctacttctttctggatggtatggtgaggttttattgtctttttgtcatgaaataatgtttttaattaagagtggggtttga

SEQ ID NO: 52 - DNA, 500 bp, Escherichia coli
ttgcaaaagcaattttgcaacaaactactgcttgatacaaataaggagaatgttatggaaattcaaaacacaaaatcaacccagattttatatacagatatatccacaaaacaaactcaaagttcttccgaaacacaaaaatcacaaaattatcagcagattgcagcgcatattccacttaatgtcggtaaaaatcccgtattaacaaccacattaaatgatgatcaacttttaaagttatcagagcaggttcagcatgattcagaaatcattgctcgccttactgacaaaaagatgaaagatctttcagagatgagtcacacccttactccagagaacactctggatatttccagtctttcttctaatgctgtttctttaattattagtgtagccgttctactttctgctctccgcactgcagaaactaaattgggctctcaattgtcattgattgcgttcgatgctacaaaatcagctgcagagaacattgttcggca

SEQ ID NO: 53 - DNA, 668 bp, Escherichia coli
aagtcaaagcaggggttgcccgaacctttaaagccccaaacctgtatcaatccagtgaaggctatctgctctactcgaaaggcaatggctgtccaaaagatattacatcaggcgggtgctacctgatcggtaataaagatctcgatccggaaatcagcgtcaataaagaaattggactggagttcacctgggaagattaccacgcaagtgtgacctacttccgcaatgattaccagaataagatcgtggccggggataacgttatcgggcaaaccgcttcaggcgcatatatcctcaagtggcagaatggcgggaaagctctggtggacggtatcgaagccagtatgtctttcccactggtgaaagagcgtctgaactggaataccaatgccacatggatgatcacttcggagcaaaaagacaccggtaatcctctgtcggtcatcccgaaatatactatcaataactcgcttaactggaccatcacccaggcgttttctgccagcttcaactggacgttatatggcagacaaaaaccgcgtactcatgcggaaacccgcagtgaagatactggcggtctgtcaggtaaagagctgggcgcttattcactggtggggacgaacttcaattacgatattaataaaaatctgcgtcttaatgtcggcgtc

SEQ ID NO: 54 - DNA, 1689 bp, Escherichia coli
gcgatgtttaaccccgattcggcgcagctggacaatatggcctgggcgcagccggcgattgtcgcgtttgaaatcgcgatggcggcgcactggcacgctgaaggactgaagccagacttcgccattgggcattccgtcggtgaatttgccgctgccgtcgtctgcggacactatacgattgaacaggtcatgccactggtttgtcgacgcggcgcactgatgcagcagtgcgcaagcggcgcaatggtggcggtatttgcagacgaagacacgctgatgccgctggctcgccagtttgagctggatctcgccgccaacaacggtacgcaacatacggtattttccgggccggaagcccgtctcgcggtattttgcaccacgctctcgcagcataacattaactatcgtcgcctgagcgtaaccggcgcggcgcactccgctttactggaaccgatactcgatcggttccaggacgcctgcgcggggctgcacgcggagccggggcaaataccgattatttccacgctcaccgccgacgtcattgatgagtcaacgctcaaccaggcggattactggcgccgacacatgcgccagccggtgcgttttatccagagtattcagatggcgcatcagctcggcgcccgcgtttttctggagatggggcccgatgcccagttggttgcttccgggcagcgcgaataccgcgataacgcatactggatagccagcgcccggcgtaacaaagaggcgagcgatgtcctcaatcaggccctgctccagctttacgctgccggtgtcgccttaccgtggaccgacctactggcgggtgatggacaacgtatcgctgcgccatgttatccgtttgatactgagcgttactggaaagagcgcgtctccccggcctgcgaacctgccgacgcagcgctgtctgccgggctggaggtggcgagtcgcgccgcgacagcgctcgatctcccccgtctggaagcgcttaaacagtgcgccacgcgactgcacgccatctacgtcgatcaactggtacaacgctgtaccggcgatgccattgaaaacggcgtggacgccataaccatcatacgccgtggacgtctgctgccccgctaccagcagctactccagcgcctgctgaataactgcgtggtcgacggcgattaccgctgcaccgacgggcgatacgtccgcgcccaccccattgaacatcaacagcgggaatcactgctgacggaacttgccggttattgtgaaggttttcaggctattcccgacaccatcgcccgtgccggcgatcggttatatgacatgatgagcggcgcggaagaaccggtggcgattatcttcccgcaaagcgcctccgacggcgtggaagtgctgtatcaggaattcagctttggccgctatttcaaccaaatcgccgccggggtattacgcggcattgtccagacgcgtcagccccgccagtcgttgcgtattcttgaagttggcggcggaaccggcggcaccaccgcgtggctgctgccggaactcaacggcgttccggcgctggagtaccacttcaccgatatctcagcgctgttcacccgccgcgcccagcagaaattcgccgactatgattttgtgaagtatagcgagctggatctcgaaaaagaggcgcagtctcagggtttccaggca

SEQ ID NO: 55 - DNA, 1241 bp, Escherichia coli
gccggaaagcctggcctttaaccatccggccagcgccccgtatattcaggaactggcgacaatttgccaacagcttgcacagcgcttacagcgcccggtacgcctgcttgaggtgggaacccgcaccggccgcgccgcagaatcgctgttggcacagctcaacgccggacagattgagtatgtcgggcttgagcagagccaggagatgctactgagcgcccggcagaggctcgcctcctggcctggtgcccgtctgtccccctggaatgcagacacgctggcggcgcacgctcactcgggggacattatctggcttaataacgccctgcatcgtctgctgccggaagatcccgggctccttgcgacattacaacagcttgccgttcccggcgcgctgctctacgtgatggagtttcgccagttaacgccgtccgccctgctcagcacgctcctgttaaccaatgggcagccggaggccttgctgcataacagcgccgactgggcggcattatttagcgcggccgccttcaactgtcagcatagcgatgaggtcgcggggttacaacgcttcctcgtacaatgtcctgacaggcaggtgcgccgcgatccccgtcaacttcaggccgccctcgccgggcgtctgccggggtggatggtgccgcaacggatcgtcttcctcgacgccttaccgctgacggctaacgggaaaattgactaccaggcgctgaagcgtcgtcatacccctaaagcggaaaaccaggccgaagcggatttaccccagggcgacattgaaaaacaggttgccgccctctggcagcaactcttatcgactggcaatgtcaccagagaaaccgacttcttccagcaaggcggcgatagcctgctggcgacccgtctgaccgggcaacttcatcaggcaggttatgaagcgcaattaagcgacctgtttaatcatccccggctggcggattttgccgccacgctgcgtaaaatcgacgtcccggtcgaacaaccattcgtccactctcctgaagaacgctaccagccctttgcgcttaccgacgtgcagcaggcttacctggtggggcgtcagccgggctttaccctgggcggcgtcggctcacatttctttgttgaatttgaaattgccgatctggacctcacccggctggagacggtctggaaccgattaatcgcccgccacgatatgctacgcgccgtcgtgcttgatggacagcaac

SEQ ID NO: 56 - DNA, 607 bp, Escherichia coli
tcacataggattctgccgtttttaacaatgcaggataataagatgaaaaaaatgttattttctgccgctctggcaatgcttattacaggatgtgctcaacaaacgtttactgttggaaacaaaccgacagcagtaacaccaaaggaaaccatcactcatcatttcttcgtttccccaattggacagagaaaactgttgatgcagccaaaatttgttggcggtgcagaaaatgttgttaaaacagaaactcagcaaacattcgtaaatgcattgcccggttttatcacttttggcatctatactccgcgggaaacccgtgtatattgctcacaataggcccatcgatatggggagctcatctgcactgttcattataacttctgggctccctacagttgtttttgcatagtgataagcctctctctgagggaggaaataatcctgttcagcgatgtctaccagtcgggggggctgcattatccaccccgaggcggtggtggcttcacgcggggatgggcagattgatctgatatgcaaccgacgacgaccagcggcaacatcatcacgcagagcttcattttcagatttgggccaccttttgatttct

SEQ ID NO: 57 - DNA, 778 bp, Escherichia coli
aagtgtcgattttattggtgtagggacagggccatttaatctcagcattgctgcgttgtcacatcagatcgaagaactggactgtctcttctttgatgaacatcctcatttttcctggcatccgggtatgctggtaccggattgtcatatgcagaccgtctttctgaaagatctggtcagtgctgttgcacctacaaatccctacagttttgttaactatctggtgaagcacaaaaagttctatcgcttccttacaagcagactacgtacagtatcccgtgaagagttttctgactacctccgctgggctgctgaagatatgaataacctgtatttcagtcataccgttgaaaacattgatttcgataaaaaacgtcgattgtttctggtgcaaaccagccagggacaatattttgcccgcaatatctgccttggtacaggaaaacaaccttatttaccaccctgtgtgaagcatatgacacaatcctgtttccatgccagtgaaagtaatcttcgtcggccggatcttagtggaaaacggataaccgtggttggtggaggacagagtggtgcagacctgttccttaatgcattacgcggggaatggggagaagcggcggaaataaactgggtgtcccggcgtaataattttaacgcactggatgaggctgcttttgctgatgattattttacacctgaatatatttcaggcttctccggactggaggaagatattcgccatcagttactggatgagcagaaaactgacatcggatgg

SEQ ID NO: 58 - DNA, 302 bp, Escherichia coli
ggctggacatcatgggaactggtacgctgaacatcgatgaatcccggcagcttcagttgatcacacagtactataaaagccagggcgacgacgattacgggcttaatctcgggaaaggcttctctgccatcagagggaccagcacgccattcgtcagtaacgggctgaattccgaccgtattcccggcactgacgggcatttgatcagcctgcagtactctgacagcgcttttctgggacaggagctggtcggtcaggtttactaccgcgatgagtcgttgcgattctacccgttcccgacg

SEQ ID NO: 59 - DNA, 2126 bp, Escherichia coli
cttcctgttctgattcttctggcgctatcggggagcttttctaccgctgtagccgctgataaaaaagagactcaaaatttctactatccagaaacactggatttaactcctctgagattacacagccctgaatcaaatccctggggggctgattttgattatgccaccagatttcaacagctggatatggaggctctgaaaaaagatatcaaagatttgctgacaacttcccaggattggtggcctgcggattatggtcattatggtcctttctttattcgtatggcttggcacggtgccggaacatacaggacatatgatggccggggaggcgccagtggtggtcagcaacgttttgaaccgctgaacagctggccggataacgttaatctggataaagcccgtcgattgctgtggccagtcaagaaaaaatacggctccagtatttcctggggagacctgatggtcctgactggtaatgttgcccttgaatccatgggatttaaaacgctgggatttgctggcggaagagaagatgactgggagtcggacctggtatactgggggcctgacaacaagcctcttgcagataaccgggataaaaacgggaaacttcagaaacctcttgccgccacgcagatgggacttatttatgtcaatcctgaaggccccggtggaaaaccagatcctctggcttccgcgaaagatatcagggaagctttttcacgtatggccatggatgatgaggagactgtggccctgatcgcgggagggcatacatttggtaaagcacatggtgcagcgtctcctgaaaaatgtattggcgcagggcctgatggtgcacctgtggaggagcagggactgggatggaaaaataaatgtggtacaggaaacggcaaatataccatcaccagtggcctggaaggagcctggtcgacatcgccaacccagttcacaatgcagtatctgaagaatttatataaatatgaatgggagctgcacaagagtcctgccggtgcttatcagtggaagcctaaaaaagcggcaaatatagttcaggacgcgcatgatccgtctgtcctgcatccgttgatgatgtttacgacggatattgctcttaaagttgatcctgaatataagaaaataaccacccgtttcctgaatgatccaaaagcttttgagcaggcattcgcaagagcatggtttaaactgacccaccgggatatgggaccggcagcccgatatcttggtaatgaagttcctgcagaatcatttatctggcaggatcctcttcctgcggcggattatacaatgattgatggtaaagacattaagtcgctgaaagagcaggttatggatttgggtatccctgcatctgagctgataaagacagcctgggcttcagcttccacatttcgtgtgactgattatcgtgggggaaataatggtgcccgcatcaggttacagcccgaaattaactgggaagttaatgagcctgaaaaactgaagaaagtactggcatccctgacctcattacagcgtgaatttaacaaaaaacagtctgacggaaagaaagtgtcgttggctgatttaattgttctttcgggtaatgctgcaatcgaagatgcggccagaaaagccggggtggaacttgagattccctttactccgggaagaactgacgcctctcaggagcagacggatgttgcctcattcagtgtactggagccgacagcagatggattcagaaattattactcaaaaagcagaagtcatatatcgccggttgaaagcctcattgataaagccagtcagctggatctcaccgttcctgaaatgacggcattactgggtggtctgcgggtaatggatattaatacaaataattcttcgttgggagtgtttaccgatacccctggtgttctggataacaagttttttgttaatctgctggatatgtcaacacgatggagtaaagcagataaagaagatacatacaatggattcgatcgtaaaacgggagcattaaaatggaaagcatcctctgttgatttaatcttcagttcaaatcctgaattacgtgcggtggcagaagtatatgcctcggatgatgcgagaaataagtt

SEQ ID NO: 60 - DNA, 501 bp, Escherichia coli
aattgttttaaaatctgttctttttctgatattgcctgagtgagttttgattctttttcgcttatctcttcttcatatttctgtatgtcatttatttttttttcaatatcatctggatatttttcttgtctcacaacatcgttgtgatttatttttgaaagtttaattacctcttccttttctttttttagctcattaatcctgttcaataataaaaattgtcttttcttaaatatagatttagtcaattcaatatctgcatccttcttttttgactcttgcaccaaatgagatacaatattgaagatagaatttctctcttcgactacctcccataatgcagcctcagccgaaacgttatctttcgatgtttctatataaggtaggtttgcataggcttgtaactcattataaagcttcagaacctcggttctttcttttattaattcagatactaagtactcactaacctttgtatcattaaatgtaatttcagtctca

SEQ ID NO: 61 - DNA, 270 bp, Escherichia coli
gcgcatttgctgatactgttgggcatttttggttacattatgcaccgcacgatgccagacatctcattcccggtgtttttacttaatggcctgattcccttttttatctttagcagtatcagcaatcgttctgtaggcgctattgaagcgaaccaggggttgtttaattatcgaccagtaaaacccatcgatacgatcattgcacgcgcactgcttgagacgctgatttacgttgctgtttatatattgctcatgcttatcgtctggatg

SEQ ID NO: 62 - DNA, 390 bp, Escherichia coli
tcctcttgctactattccccctcaatatcagcattggtttttatggaatccacttgtgcatgctgtagaactaatccgaagggcatggatatctggttatcgtagtcctgatgtaagttgggcgtatctgtcggttgtcaccttattattgctcacttttgctatgagttgttaccgattacggcatcgccaattgattgctagttagcgttaagaaaaatgattattcttgataatgtatcaaaatattatccgactaaatttggacgaaattatgtcctgaggaatgtaaatattgagctaccaagggaccgtaatataggtattctaggtatcaatggagcaggaaaatctactttgttacgtttgttaggagggatggatacgcct

SEQ ID NO: 63 - DNA, 659 bp, Escherichia coli
cagttcgctcgtaaagcagaaaaatgcgacagaagatgttgttttaataggcaaaatgattttagatgaagttagaagttacagaactatacataatgatcgaaatatcgtaagtaactcaggaaactggaaaacatcttttttatgtaatcttgctagactactatatagcatatttaatggtagtaactatttttgttcccgagagggtgaaaataattcatcccccagttctactctacttactatacatcagcctgaaaagcaggaactattacaacaaaagagtatcaaacatttaccaacaagtaataacatcgacggatacattaaaataagaaaaacaagaggcgctgaagatcaaacaacaactatcactcaaagtttgataattaatgagttgttaaatggagttgatagaaataccatcccttttcagaaaataagtgagctcaatgatatcatacattcatatgaaaatatgcaaattaaaaatagtcgaaaaggtatagaaatacttgttaagcagggagagctgttatcatcattaataaatgataataaaggaaataaacaattatcagacaatgcatctaaaataataaacttattgggtatagagtatcagtcacataaagtagacatagagccatttatac

SEQ ID NO: 64 - DNA, 501 bp, Escherichia coli
gaacaattcaaacagttcagtattgaaaaacaggctgcgattaactcgctattacagttgcgcggaatgttagaaatgctgggagagatggggataaacatcagcgacgatttacaaaaagtcacttctgcaattaatgccatcgaatctgatgtcctgcgtattgctctgttgggggcgttctccgatggcaaaaccagcgttatcgccgcatggctgggtaaagtaatggatgatatgaatatttccatggatgagtcctccgatcggttgagtatttacaaaccggaaggtctgccagatcagtgtgaaattgttgatacgcccgggctgtttggtgataaagagcgcgaggtggacgggagactggtgatgtatgaagacctgaccagacgctatatatctgaagcacacctgattttttacgtggttgatgccacgaacccgctcaaggagagccacagcgacatcgtaaaatgggtattgcgcgatttgaataaa

SEQ ID NO: 65 - DNA, 424 bp, Escherichia coli
caaatacagtccgcgtacgaatgaaagatgcttatcaacgtgatggtaaatatccagattttgtggacccattaagccttactgcaaatacaattaaaactgatacaagcggaatacctgcagcacagttagttcagcttgggaaaattacaccagacgaagtgcgtaataacatttctggcgactttatcgctattggcggtgctttaacttcgaatggtgctcaagttaaaaaaggttttgctatcgaacttaatggattaagccaagagcagtgccgttctattcttgggcaagttgggaataactgggaatatgttgctattggtacttctgcgtctggttcatatgccatgacagcaactggtgtagatatgtctgtggccgcctctacaactgttttacgctctttaggtaacaatgg

SEQ ID NO: 66 - DNA, 275 bp, Escherichia coli
ttacggcgttactatcctctctatgtgcatacggagctccccagtctattacagaactatgttcggaatatcgcaacacacaaatatatacgataaatgacaagatactatcatatacggaatcgatggcaggcaaaagagaaatggttatcattacatttaagagcggcgcaacatttcaggtcgaagtcccgggcagtcaacatatagactcccaaaaaaaagccattgaaaggatgaaggacacattaagaatcacatatctgaccgagacc

SEQ ID NO: 67 - DNA, 500 bp, Escherichia coli
ttggcagttacaggaatgcattgtgataatgcgtatggaaatacaatacatattatagaacaagataattttaatattatcaaggttgtggatataaatatcaatacaacttcacatactcacattctccattcaatgagtgtttgcctcaattcgtttggtgattttttttcaaataacacatatgatgcggttatggttttaggcgatagatatgaaatattttcagtcgctatcgcagcatcaatgcataatattccattaattcatattcatggtggtgaaaagacattagctaattatgatgagtttattaggcattcaattactaaaatgagtaaactccatcttacttctacagaagagtataaaaaacgagtaattcaactaggtgaaaagcctggtagtgtgtttaatattggttctcttggtgcagaaaatgctctttcattgcatttaccaaataagcaggagttggaactaaaatatggttcactgtt

SEQ ID NO: 68 - DNA, 537 bp, Escherichia coli
gcttactgattctgggatggattaacagaacacaactggcttgtccataagcaaaatgaaggcaaaaaaatatgaaaatcaaatatacaatgaaaatggccgccgttgccagcgtcatggtcgccggctagctatcgcggatgcaaatgggctcaacactgtgaacgccggggatggcaagaatctgggcaccgcaaccgcgacgatcaccactctgcagagctgctctgtcgacctgaatctcgttaccccgaacgcgacagtgaacagagcaggaatgctagcaaaccgcgaaatcactaaattttcggtggggagtaaggattgccctagcgacacctatgctgtatggtttaaagagatcgatggcgaaggacagggggtcgcgcagggcactacggtgaccaacaagttttaccttaaaatgacatcggccgacgggaccgcgagcgtaggggacatcaacataggaaccaaatcaggcaaaggcctgagtggtcaactggtagggggaaaattcgacggaaaaataacggt

SEQ ID NO: 69 - DNA, 1422 bp, Escherichia coli
cttgcggaggcttgtctgagcggtttccgcgattctcttctgtaaattgtcgctgacaaaaaagattaaacataccttatacaagacttttttttcatatgcctgacggagttcacacttgtaagttttcaactacgttgtagactttacatcgccaagggtgctcggcataagccgaagatatcggtagagttaatattgagcagatcccccggtgaaggatttaaccgtgttatctcgttggagatattcatggcgtattttggatgataacgaggcgcaaaaaatgaaaaagacagctatcgcgattgcagtggcactggctggtttcgctaccgtagcgcaggccgctccgaaagataacacctggtacactggtgctaaactgggctggtcccagtaccatgatactggtttcatcaacaacaatggcccgacccatgaaaaccaactgggcgctggtgcttttggtggttaccaggttaacccgtatgttggctttgaaatgggttacgactggttaggtcgtatgccgtacaaaggcagcgttgaaaacggtgcatacaaagctcagggcgttcaactgaccgctaaactgggttacccaatcactgacgacctggacatctacactcgtctgggtggcatggtatggcgtgcagacactaaatccaacgtttatggtaaaaaccacgacaccggcgtttctccggtcttcgctggcggtgttgagtacgcgatcactcctgaaatcgctacccgtctggaataccagtggacgaacaacatcggtgacgcacacaccatcggcactcgtccggacaacggcatgctgagcctgggtgtttcctaccgtttcggtcagggcgaggcagctccagtagttgctccggctccagctccggcaccggaagtacagaccaagcacttcactctgaagtctgacgttctgttcaacttcaacaaagcaaccctgaaaccggaaggtcaggctgctctggatcagctgtacagccagctgagcaacttggatccgaaagacggttccgtagttgttctgggttacaccgaccgcatcggttctgacgcttacaaccagggtctgtccgagcgccgtgctcagtctgttgttgattacctgatctccaaaggtatcccggcagacaagatctccgcacgtggtatgggcgaatccaacccggttactggcaacacctgtgacaacgtgaaacagcgtgctgcactgatcgactgcctggctccggatcgtcgcgtagagatcgaagttaaaggtatcaaagacgttgtaactcagccgcaggcttaagttctcgtctggtagaaaaacgctgctgcgggtttttttttgcctttagtaaattgaactgactttcgtcagttattccttacccagcaatgcct

SEQ ID NO: 70 - DNA, 559 bp, Escherichia coli
atctagccgaagaaggaggccgaaaagtcagtcaactcgactggaaattcaataacgctgcaattattaaaggtgcaattaattgggatttgatgccccagatatctatcggggctgctggctggacaactctcggcagccgaggtggcaatatggtcgatcaggactggatggattccagtaaccccggaacctggacggatgaaagtagacaccctgatacacaactcaattatgccaacgaatttgatctgaatatcaaaggctggctcctcaacgaacccaattaccgcctgggactcatggccggatatcaggaaagccgttatagctttacagccagaggtggttcctatatctacagttctgaggagggattcagagatgatatcggctccttcccgaatggagaaagagcaatcggctacaaacaacgttttaaaatgccctacattggcttgactggaagttatcgttatgaagattttgaactcggtggcacatttaaatacagcggctgggtggaatcatctgataacgatgaacactatgacccggg

SEQ ID NO: 71 - DNA, 360 bp, Escherichia coli
atgaggaacataatggcaggttttttaatattcctgtcttctgctgcttatgctgatatcaatctgtatggtcctggtggcccgcatacagccttgcttgatgcagccaaactttatgccgaaaaaacaggtattatagtgaacgttcattacggcccacagaacaaatggaatgaagatgccaaaaaaaatgcagatatcttgtttggcgcatcagaacaatctgctctggctatcattcgggaccataaagacagcttcagtgaaaaagatattcagcctctttatctgcgaaaaagtattttactggtaaagaaaggtaatcctaaaaatatccggagtattgacgacctgaccaga

SEQ ID NO: 72 - DNA, 721 bp, Escherichia coli
atggcagtggtgtcttttggtgtaaataatgctgctccaactattccacaggggcagggtaaagtaacttttaacggaactgttgttgatgctccatgcagcatttctcagaaatcagctgatcagtctattgattttggacagctttcaaaaagcttccttgaggcaggaggtgtatccaaaccaatggacttagatattgaattggttaattgtgatattactgcctttaaaggtggtaatggcgccaaaaaagggactgttaagctggcttttactggcccgatagttaatggacattctgatgagctagatacaaatggtggtacgggcacagctatcgtagttcagggggcaggtaaaaacgttgtcttcgatggctccgaaggtgatgctaataccctgaaagatggtgaaaacgtgctgcattatactgctgttgttaagaagtcgtcagccgttggtgccgctgttactgaaggtgccttctcagcagttgcgaatttcaacctgacttatcagtaatactgataatccggtcggtaaacagcggaaatattccgctgtttatttctcagggtatttatcatgagactgcgattctctgttccacttttcttttttggctgtgtgtttgttcatggtgtttttgccggtccgtttcctccgcccggcatgtcccttcctgaatactggggagaagagcacgtatggtgggacg

SEQ ID NO: 73 - DNA, 318 bp, Escherichia coli
gacggctgtactgcagggtgtggcggttggattgtcagcctcaaggtctaaatatctggggcgtgataacgattctgcttacctgcgtatatccgtgccgctggggacggggacagcgagctacagtggcagtatgagtaatgaccgttatgtgaatatggccggctacactgacacgttcaatgacggtctggacagctacagcctgaacgccggccttaacagtggcggtggactgacatcgcaacgtcagattaatgcctattacagtcatcgtagtccgctggcaaatttgtccgcgaatattgcatccctgca

SEQ ID NO: 74 - DNA, 336 bp, Escherichia coli
gcaacagcaacgctggttgcatcatattcgtaatagtatcaactaaaatacgttaattttatatctcgtaaaataaaatgttttctgtaccgctctccggagggggaatgattcgtttatcattatttatatcgttgcttctgacatcggtcgctgtactggctgatgtgcagattaacatcagggggaatgtttatatccccccatgcaccattaataacgggcagaatattgttgttgattttgggaatattaatcctgagcacgtggacaactcacgtggtgaagtcacaaaaaccataagcatatcctgtccgtataagagtggctctctct

SEQ ID NO: 75 - DNA, 461 bp, Escherichia coli
tcgtgctcaggtccggaatttgcgagtggagtgtattttcaggagtatctggcctggatggttgttcctaaacatgtctatactaatgaggggtttaatatatttcttgatgttcagagcaaatatggttggtctatggagaatgaaaatgacaaagatttttacttctttgttaatggttatgaatgggatacatggacaaataatggtgcccgtatatgtttctatcctggaaatatgaagcagttgaacaataaatttaatgatttagtattcagggttcttttgccagtagatctccccaagggacattataattttcctgtgagatatatacgtggaatacagcaccattactatgatctctggcaggatcattataaaatgccttacgatcagattaagcagctacctgccactaatacattgatgttatcattcgataatgttgggggatgcca

SEQ ID NO: 76 - DNA, 190 bp, Escherichia coli
gggatgagcgggcctttgatgcaggtaatttgtgtcagaaaccaggagaaacaactcgtctgactgagaaatttgacgatattatttttaaagtcgccttacctgcagatcttcctttaggggattattctgttacaattccatacacttccggcatacagcgtcatttcgcgagttacttgggggcccg

SEQ ID NO: 77 - DNA, 268 bp, Escherichia coli
taagctatgtggcctgcaatggatttacctggactcatggtctttactggtctgagtattttgcatggctggttgttcctaaacatgtttcctataatggatataatatatatcttgaacttcagtccagaggaagtttttcacttgatgcagaagataatgataattactatcttaccaagggatttgcatgggatgaagcaaacacatctggacagacatgtttcaatatcggagaaaaaagaagtctggcatggtcatttggtgg

SEQ ID NO: 78 - DNA, 922 bp, Escherichia coli
tcgccaccaatcacagccgaaccgccgattggcgtaaagcggaaaactgacgtcaccagatggtttaagccaaaaggaatcgtcacgcgttcggcaactgcatagaagaaataaccaacaggaccggaagttgaaatccagtgtccaatgagcatgaaaagattgaaaaacggcggccagataaaaggaatgatcagaccaaatccactcatcacaatcagtgtaatgataggcaccagacgtgggccgctataagaacctaacgattcaggaatgcgtaaattaacgatctttttatacatgctggcgactaataacccagcaacaattccccccaacacgctggtattgtaggactggatccccagaatgatggtttgcccatgtgtcgacatttggtcagcaacgaccaataagtcgtgctgtttaagataaaagttcgttcccagatgcatcgccataaaaccaattaagccagaaaaagcaccataggctttatcctctttatcttttaataatcctaagggaatcgctatcgcaaacaatacaggtaaattaacaaaggcaaacaaaccaagactaacaatgaaatcaagtatggttttaattattggaatagccagaaatggaattaactttgccatatcatcactggctaaaccacttcccagccctagcatcatgccacatacacttagcagagcaatgggatacataaatgccttccccaggctctgaaaaaaactccaggctttcttttgtttcatgtgggttatctcatataaatgttatatataattagtccattaatactttggtacgaatagagagatatagtttttcttctaaaattaattcatatttaaaagtggcatacagataccgttcaatttcatgaattgcgcgctgtaacaggatgtcc

SEQ ID NO: 79 - DNA, 501 bp, Escherichia coli
ggtgatcgattattccgctgagcgtattcagtctttaaaagacaaatacagcctgccggatgagtttatcttgtcgctggcgatgatcgagccgcggaaaaatattgaagcgcttattcacgcctacagcttgctgcctgccgagctgcagcagcgctatccgatggtgctggcgtataaagtgcagccagaacaactggagcggatcctgcgtctggcggaaagctatggtttgtcacgcagccagcttatctttactgggttcctgaccgacgacgatctgattgccctgtacaacctgtgcaaactgtttgtgttcccgtcgctgcatgaaggtttcggcctgccgccgctggaagcgatgcgctgcggggcggcgaccttaggttcaaacattaccagcctgccggaagtcattggctgggaagatgccatgttcaatccgcatgatgtgcaggacattcgccgggtcatggagaaggcgctgaccgatgaggcgtt

SEQ ID NO: 80 - DNA, 500 bp, Escherichia coli
tctgcacgtttaaaattattgcctgggttaaagtcaactgagtatgtgtattcagatcttcatgctttacttgatactaatggtgggagttccttaggtccgaacattggtagtgatggttctaacctaacaataacatgtttatcgatgagcagagtttttcttactgaaaaacttgttaattctatatatcagcatataccttattttaaaggtgatattctgattgttgataatggcagcacagtagaagaactttcaattttacaagatttaagtgataggatcccgttaaatattagagttgtcgagcttggtaataattttggcgtaagtggtggaagaaacaaaactttagagcatataaaaacagaatgggcaatgtttctcgataatgatatttatttcataaataatccacttccgagattgcaaaatgatatttcaagacttggttgtcattttatcaatatgccattgcttgattctgacggagaaac

SEQ ID NO: 81 - DNA, 406 bp, Escherichia coli
tagagaaattatcaagttagttccattagtatcaattgatctgctaattgaaaacgagaatggtgaatatttatttggtcttaggaataatcgaccggccaaaaattatttttttgttccaggtggtaggattcgcaaaaatgaatctattaaaaatgcttttaaaagaatatcatctatggaattaggtaaagagtatggtatttcaggaagtgtttttaatggtgtatgggaacatttctatgatgatggttttttttctgaaggcgaggcaacacattatatagtgctttgttacacactgaaagttcttaaaagtgaattgaatctcccagatgatcaacatcgtgaatacctttggctaactaaacaccaaataaatgctaaacaagatgttcataactat

SEQ ID NO: 82 - DNA, 292 bp, Escherichia coli
gtgtccatttatacggacatccatgtgatatggaacaaattgtagaactggccaaaagtagaaatttgtttgtaattgaagattgcgctgaagcctttggttctaaatataaaggtaaatatgtgggaacatttggagatatttctacttttagcttttttggaaataaaactattactacaggtgaaggtggaatggttgtcacgaatgacaaaacactttatgaccgttgtttacattttaaaggccaaggattagctgtacataggcaatattggcatgacgttatagg

SEQ ID NO: 83 - DNA, 259 bp, Escherichia coli
cggacatccatgtgatatggaacaaattgtagaactggccaaaagtagaaatttgtttgtaattgaagattgcgctgaagcctttggttctaaatataaaggtaaatatgtgggaacatttggagatatttctacttttagcttttttggaaataaaactattactacaggtgaaggtggaatggttgtcacgaatgacaaaacactttatgaccgttgtttacattttaaaggccaaggattagctgtacataggcaa

SEQ ID NO: 84 - DNA, 786 bp, Escherichia coli
atccatcaggaggggactggataggttattttctccattatgactgcatggttaatgagcagtgtaataatggttttataatgtttgaacctggatatgaattaattgtttccttatttggatatttgggatttcagacaattattatttttatagccgctgtaaatgtaattctaatattaaattttgcaaagcattttgaaaacggaagttttgttattgttgcgataatgtgcatgttcctttggagtgtttatgttgaggcgattagacaggctctggccttatctatagttatatttgggattcattctctttttttgggtagaaaaaggaaatttataacattagtattatttgcgtcaactttccatataactgctttgatttgttttcttctaatgactcctctattttcaaagaaattaagcaagataataagttatagcctattaattttcagtagcttctttttcgctttttctgaaaccatattaagtgcactccttgcaattttgccagaaggatccattgccagtgaaaaattaagtttttacttagcaaccgagcaatacaggccacagttatctattgggagtggcactattcttgacattatacttatttttctgatatgtgtaagttttaaacgaataaagaaatatatgctcgctaattataatgctgcaaatgagatattgcttattggttgctgtctttatatttctttcggtatttttatcgggaaaatgatgccagttatgactcgcattggttggtatggtt

SEQ ID NO: 85 - DNA, 521 bp, Escherichia coli
ctaccgtagcgggcgatggtagctggacaaccaccgtacccgccgccgatctcagcgtgttacgcgacggcgacgccaccgtgcaggccagcgtcagcactattaacggcaacacggcttcggcaacccacgcctacagcgtcgatgccacggccccgacgcttgccattaacaccatcgccaccgacgatattctgaacgctgccgaggcgggcaatccgttaaccatcagcggtagcagcaccgccgaagcggggcagacggtaaccgtcacgcttaatggtgtgacttacagcggctccgtccaggcggacggcagctggagcgtcagcttaccgacggcggatctcagcaatctgaccgccagccagtacaccgttagtgcctcggtaagcgataaagcgggtaacccggcgtccgctaaccacgggctggcggtggatctcaccgtgccggtgctgaccatcaacaccgtctccggcgatgacattattaacgccgccgaacacggacaggcgctg

SEQ ID NO: 86 - DNA, 408 bp, Escherichia coli
ctccggagaactgggtgcatcttacccgcggagatatgaaactgcatatgcaggcgaggtataaggccacacattatcccgtcgccgggggaaaggcaaatggacaggtatggttttctctgacctatctgtaactggcagatataatgccatttaattaaggctgttaataacatgatgaagcacatgcgtatatgggccgttctggcatcatttttagtctttttttatattccgcagagctatgccggggttgctctgggtgccacccgtgagatttaccctgaagggcaaaaacaggtacaactggcggtaacaaataatgatgataaaagtagttaccttattcagtcatggattgaaaatgctgaaggaaaaaaggatgccaggtttgtaattactcctccg

SEQ ID NO: 87 - DNA, 500 bp, Escherichia coli
ccctgaccttgggtgttgcgacaaatgcgtctgctgtcaccacggttaatggtggtacagttcattttaagggggaagttgttgatgctgcatgtgctgtaaacactaattcagcaaatcaaacgttttctgggcaagttcgttcagctaagttggcgaatgatggagagaagagttcccctgttggatttagtattgaacttaatgactgtagttctgcaactgccgggcatgcatcaattatctttgcaggaaatgttattgctacacacaatgatgtgctgtctctacagaatagtgctgcaggtagtgcaacaaatgtaggtattcagatattggatcatacaggtactgcagttcaatttgacggagtgactgcatctacacaatttacattaacagatggcaccaataaaattcctttccaggcagtttattatgcaacaggtaagtcaacgcctggtattgccaacgccgacgccacctttaaagttcagtac

SEQ ID NO: 88 - DNA, 214 bp, Escherichia coli
aagaaatcaatattatttatttttctttctgtattgtctttttcacctttcgctcaggatgctaaaccagtagagtcttcaaaagaaaaaatcacactagaatcaaaaaaatgtaacattgcaaaaaaaagtaataaaagtggtcctgaaagcatgaatagtagcaattactgctgtgaattgtgttgtaatcctgcttgtaccgggtgctatt

SEQ ID NO: 89 - DNA, 163 bp, Escherichia coli
tcccctcttttagtcagtcaactgaatcacttgactcttcaaaagagaaaattacattagagactaaaaagtgtgatgttgtaaaaaacaacagtgaaaaaaaatcagaaaatatgaacaacacattttactgctgtgaactttgttgtaatcctgcctgtgc

SEQ ID NO: 90 - DNA, 368 bp, Escherichia coli
gcaataaggttgaggtgattttatgaaaaagaatatcgcatttcttcttgcatctatgttcgttttttctattgctacaaatgcctatgcatctacacaatcaaataaaaaagatctgtgtgaacattatagacaaatagccaaggaaagttgtaaaaaaggttttttaggggttagagatggtactgctggagcatgctttggcgcccaaataatggttgcagcaaaaggatgctaatatatttatcaatagcattcagcaccatatacacaaaaataatttttcataaaaagaactctataaaataaatattttttgtgacaatgtcctaacgcaagacggacattgtccatttctcactgcaggc

SEQ ID NO: 91 - DNA, 583 bp, Escherichia coli
acactggatgatctcagtgggcgttcttatgtaatgactgctgaagatgttgatcttacattgaactggggaaggttgagtagtgtcctgcctgattatcatggacaagactctgttcgtgtaggaagaatttcttttggaagcattaatgcaattctgggaagcgtggcattaatactgaattgtcatcatcatgcatcgcgagttgccagaatggcatctgatgagtttccttctatgtgtccggcagatggaagagtccgtgggattacgcacaataaaatattgtgggattcatccactctgggggcaattctgatgcgcagaactattagcagttgagggggtaaaatgaaaaaaacattattaatagctgcatcgctttcatttttttcagcaagtgcgctggcgacgcctgattgtgtaactggaaaggtggagtatacaaaatataatgatgacgatacctttacagttaaagtgggtgataaagaattatttaccaacagatggaatcttcagtctcttcttctcagtgcgcaaattacggggatgactgtaaccattaaaactaatgcctgtc

SEQ ID NO: 92 - DNA, 1612 bp, Escherichia coli
agtcctcgatggcggtccattatctgcattatgcgttgttagctcagccggacagagcaattgccttctgagcaatcggtcactggttcgaatccagtacaacgcgccatatttatttaccaggctcgcttttgcgggccttttttatatctgcgccgggtctggtgctgattacttcagccaaaaggaacacctgtatatgaagtgtatattatttaaatgggtactgtgcctgttactgggtttttcttcggtatcctattcccgggagtttacgatagacttttcgacccaacaaagttatgtctcttcgttaaatagtatacggacagagatatcgacccctcttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgtgtatacgatgacgccgggagacgtggacctcactctgaactgggggcgaatcagcaatgtgcttccggagtatcggggagaggatggtgtcagagtggggagaatatcctttaataatatatcagcgatactggggactgtggccgttatactgaattgccatcatcagggggcgcgttctgttcgcgccgtgaatgaagagagtcaaccagaatgtcagataactggcgacaggcctgttataaaaataaacaatacattatgggaaagtaatacagctgcagcgtttctgaacagaaagtcacagtttttatatacaacgggtaaataaaggagttaagcatgaagaagatgtttatggcggttttatttgcattagcttctgttaatgcaatggcggcggattgtgctaaaggtaaaattgagttttccaagtataatgaggatgacacatttacagtgaaggttgacgggaaagaatactggaccagtcgctggaatctgcaaccgttactgcaaagtgctcagttgacaggaatgactgtcacaatcaaatccagtacctgtgaatcaggctccggatttgctgaagtgcagtttaataatgactgaggcataacctgattcgtggtatgtgggtaacaagtgtaatctgtgtcacaattcagtcagttgacagttgcctgtcagactgagcatttgttaaaaaaatttcgcatggtgaatccccctgtgtggaggggcgactggtgaaaaatccttgcttgtgattcattatcgacac

SEQ ID NO: 93 - DNA, 502 bp, Escherichia coli
gcgaaggaatttaccttagacttctcgactgcaaagacgtatgtagattcgctgaatgtcattcgctctgcaataggtactccattacagactatttcatcaggaggtacgtctttactgatgattgatagtggctcaggggataatttgtttgcagttgatgtcagagggatagatccagaggaagggcggtttaataatctacggcttattgttgaacgaaataatttatatgtgacaggatttgttaacaggacaaataatgttttttatcgctttgctgatttttcacatgttacctttccaggtacaacagcggttacattgtctggtgacagtagctataccacgttacagcgtgttgcagggatcagtcgtacggggatgcagataaatcgccattcgttgactacttcttatctggatttaatgtcgcatagtggaacctcactgacgcagtctgtggcaagagcgatgttacggtttgttactgtgacagctg

SEQ ID NO: 94 - DNA, 482 bp, Escherichia coli
cttgaacatatatctcaggggaccacatcggtgtctgttattaaccacaccccaccgggcagttattttgctgtggatatacgagggcttgatgtctatcaggcgcgttttgaccatcttcgtctgattattgagcaaaataatttatatgtggccgggttcgttaatacggcaacaaatactttctaccgtttttcagattttacacatatatcagtgcccggtgtgacaacggtttccatgacaacggacagcagttataccactctgcaacgtgtcgcagcgctggaacgttccggaatgcaaatcagtcgtcactcactggtttcatcatatctggcgttaatggagttcagtggtaatacaatgaccagagatgcatccagagcagttctgcgttttgtcactgtcacagcagaagccttacgcttcaggcagatacagagagaatttcgtcaggcactgtctgaaactgctcctgt

SEQ ID NO: 95 - DNA, 151 bp, Escherichia coli
ggtggagtatacaaaatataatgatgacgatacctttacagttaaagtgggtgataaagaattatttaccaacagatggaatcttcagtctcttcttctcagtgcgcaaattacggggatgactgtaaccattaaaactaatgcctgtcat

SEQ ID NO: 96 - DNA, 211 bp, Escherichia coli
ttctgttaatgcaatggcggcggattgtgctaaaggtaaaattgagttttccaagtataatgaggatgacacatttacagtgaaggttgacgggaaagaatactggaccagtcgctggaatctgcaaccgttactgcaaagtgctcagttgacaggaatgactgtcacaatcaaatccagtacctgtgaatcaggctccggatttgctgaa

SEQ ID NO: 97 - DNA, 226 bp, Escherichia coli
gaagaagatgtttatagcggttttatttgcattggtttctgttaatgcaatggcggcggattgtgctaaaggtaaaattgagttttccaagtataatgaggataatacctttactgtgaaggtgtcaggaagagaatactggacgaacagatggaatttgcagccattgttacaaagtgctcagctgacagggatgactgtaacaatcatatctaatacctgcagt

SEQ ID NO: 98 - DNA, 442 bp, Escherichia coli
attggtgccggtgttactgctgctcttcatcggaaaaaccaaccggcagaacaaacaatcactacacgtacggtagtcgataatcagcctacgaataacgcatctgcgcagggcaatactgacacaagtgggccagaagagtccccggcgagcagacgtaattcgaatgccagcctcgcatcgaacgggtctgacacctccagcacgggcacggtagagaatccgtatgctgacgttggaatgcccagaaatgattcactggctcgcatttcagaggaacctatttatgatgaggtcgctgcagatcctaattatagcgtcattcaacatttttcagggaacagcccagttaccggaaggttagtgggaaccccagggcaaggtatccaaagtacttatgcgcttctggcaagcagcggcggattgcgtttaggtatgggag

SEQ ID NO: 99 - DNA, 1521 bp, Escherichia coli
atgcctattggtaatcttggtcataatcccaatgtgaataattcaattcctcctgcacctccattaccttcacaaaccgacggtgcaggggggcgtggtcagctcattaactctacggggccgttgggatctcgtgcgctatttacgcctgtaaggaattctatggctgattctggcgacaatcgtgccagtgatgttcctggacttcctgtaaatccgatgcgcctggcggcgtctgagataacactgaatgatggatttgaagttcttcatgatcatggtccgctcgatactcttaacaggcagattggctcttcggtatttcgagttgaaactcaggaagatggtaaacatattgctgtcggtcagaggaatggtgttgagacctctgttgttttaagtgatcaagagtacgctcgcttgcagtccattgatcctgaaggtaaagacaaatttgtatttactggaggccgtggtggtgctgggcatgctatggtcaccgttgcttcagatatcacggaagcccgccaaaggatactggagctgttagagcccaaagggaccggggagtccaaaggtgctggggagtcaaaaggcgttggggagttgagggagtcaaatagcggtgcggaaaacaccacagaaactcagacctcaacctcaacttccagccttcgttcagatcctaaactttggttggcgttggggactgttgctacaggtctgatagggttggcggcgacgggtattgtacaggcgcttgcattgacgccggagccggatagcccaaccacgaccgaccctgatgcagctgcaagtgaaactgaaactgcgacaagagatcagttaacgaaagaagcgttccagaacccagataatcaaaaagttaatatcgatgagctcggaaatgcgattccgtcaggggtattgaaagatgatgttgttgcgaatatagaagagcaggctaaagcagcaggcgaagaggccaaacagcaagccattgaaaataatgctcaggcgcaaaaaaaatatgatgaacaacaagctaaacgccaggaggagctgaaagtttcatcgggggctggctacggtcttagtggcgcattgattcttggtgggggaattggtgttgccgtcaccgctgcgcttcatcgaaaaaatcagccggtagaacaaacaacaacaacaactactacaactacaactacaagcgcacgtacggtagagaataagcctgcaaataatacacctgcacagggcaatgtagatacccctgggtcagaagataccatggagagcagacgtagctcgatggctagcaccttgtcgactttctttgacacttccagcatagggaccgtgcagaatccgtatgctgatgttaaaacatcgctgcatgattcgcaggtgccgacttctaattctaatacgtctgttcagaatatggggaatacagattctgttgtatatagcaccattcaacatcctccccgggatactactgataacggcgcacggtta

SEQ ID NO: 100 - DNA, 446 bp, Escherichia coli
attggtgctggtgtaacgactgcgctccatagacgaaatcagccggcagaacagacaactactacaacaacacatacggtagtgcagcagcagaccggagggaataccccagcacaaggtggcactgatgccacaagagcagaagatgcttctctgaatagacgtgattcgcaggggagtgttgcatcgacacactggtcagattcctctagcgaagtggttaatccatatgctgaagttggggagcctcggaatagtctatcgactcgtcagcaagaagagcatatttacgatgaggtcgctgcagatcctgtttatagcgtcattcagaatttttcacggaatgctccagttaccggaaggttaatgggaagcccagggcaaggtatccaaagtacttatgcgcttctggcaaacagcgctggattgcgtttaggtatgggagg

SEQ ID NO: 101 - DNA, 288 bp, Escherichia coli
ggtgtggtgcgatgagcacagcaatcaagaagcgtaaccttgaggtgaagactcagatgagtgagaccatctggcttgaacccgccagcgaacgcacggtatttctgcagatcaaaaacacgtctgataaagacatgagtgggctgcagggcaaaattgctgatgctgtgaaagcaaaaggatatcaggtggtgacttctccggataaagcctactactggattcaggcgaatgtgctgaaggccgataagatggatctgcgggagtctcagggatggctgaaccgtg

SEQ ID NO: 102 - DNA, 640 bp, Escherichia coli
ggtggtgcactggagtggagctttaacagcagtaccggagctggtgcgctgacacagggaaccaccacatatgccatgcacgggcagcagggaaatgacctgaatgctggtaagaacctgatatttcaggggcagaatggtcagattaaccttaaggattcggtttctcagggggcgggttccctgacgttccgtgataattacacagtaacaacctctaacggaagtacctggaccggtgccggtattgttgtggacaacggggtgtccgtaaactggcaggttaatggtgttaagggcgataacctgcataaaattggtgaaggtacgctgacggtacagggtacaggtattaatgaaggtggcctgaaggtcggggacggaaaggttgtactgaaccagcaggcggacaataaaggacaggtgcaggcgttcagcagtgttaatattgccagtggccggccgaccgtggtactgactgatgagcggcaggtaaatccggataccgtctcatggggatatcgtgggggcacactggatgttaatggtaacagtctgacgtttcatcagttgaaggcggcagattatggtgccgtgctggcgaataacgttgataaacgggccactatcacgctggact

SEQ ID NO: 103 - DNA, 250 bp, Escherichia coli
gcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatca

SEQ ID NO: 104 - DNA, 501 bp, Escherichia coli
ctactgttcccgagtagtgtgttggcgactcaaatatggggaaaatggtcgctcagtggcgtactcagtgcaacccgcggctcttacatcggtgcgttggcatctgctttgtatattccctctgcgggcgagggcagtgctcgcgtgcccggacgtgatgagttctggtatgaggaagaactgcggcagaaagcactagcaggcagtaccgccaccacccgggtacgttttttctggggaactgacattcacggcaagcctcaggtgtatggtgttcatacgggtgaaggtacgccgtatgaaaacgtccgcgtggcgaacatgcagtggaacgagcagacgcagcgttatgaatttacccccgctcacgatgtcgatggccccctgattacctggacgccggaaaatccggaacatgggaatgttccgggccataccggtaacgacaggccgccgctggatcagcccaccattctggtgacgccgattccggacggcacc

SEQ ID NO: 105 - DNA, 22 bp, Artificial sequence
  Artificial Sequence = Primer
gcgatcatggccgcgaccagca

SEQ ID NO: 106 - DNA, 22 bp, Artificial sequence
  Artificial Sequence = Primer
caactcacccagtagccccagt

SEQ ID NO: 107 - DNA, 22 bp, Artificial sequence
  Artificial Sequence = Primer
gaaagtaaatggaatataaatg

SEQ ID NO: 108 - DNA, 23 bp, Artificial sequence
  Artificial Sequence = Primer
tttgtgttgccgccgctggtgaa

SEQ ID NO: 109 - DNA, 22 bp, Artificial sequence
  Artificial Sequence = Primer
gaaagtaaatggaatataaatg

SEQ ID NO: 110 - DNA, 23 bp, Artificial sequence
  Artificial Sequence = Primer
tttgtgtcggtgcagcagggaaa

SEQ ID NO: 111 - DNA, 21 bp, Artificial sequence
  Artificial Sequence = Primer
ggtgcaatggctctgaccaca

SEQ ID NO: 112 - DNA, 21 bp, Artificial sequence
  Artificial Sequence = Primer
gtcattacaagagatactact

SEQ ID NO: 113 - DNA, 21 bp, Artificial sequence
  Artificial Sequence = Primer
gctcacaccatcaacaccgtt

SEQ ID NO: 114 - DNA, 21 bp, Artificial sequence
  Artificial Sequence = Primer
cgttgacttagtcaggataat

SEQ ID NO: 115 - DNA, 21 bp, Artificial sequence
  Artificial Sequence = Primer
gggcccactctaaccaaagaa

SEQ ID NO: 116 - DNA, 21 bp, Artificial sequence
  Artificial Sequence = Primer
cggtaattacctgaaactaaa

SEQ ID NO: 117 - DNA, 21 bp, Artificial sequence
  Artificial Sequence = Primer
cgtgtgggagccctgagcctt

SEQ ID NO: 118 - DNA, 20 bp, Artificial sequence
  Artificial Sequence = Primer
ccggcctggttgctagtatt

SEQ ID NO: 119 - DNA, 21 bp, Artificial sequence
  Artificial Sequence = Primer
catcagttgctagtgcgaatg

SEQ ID NO: 120 - DNA, 20 bp, Artificial sequence
  Artificial Sequence = Primer
cagcaaatgtcaaatacgtt

SEQ ID NO: 121 - DNA, 21 bp, Artificial sequence
  Artificial Sequence = Primer
cgacatcgacgatctatgact

SEQ ID NO: 122 - DNA, 21 bp, Artificial sequence
  Artificial Sequence = Primer
ccaagggatattgctgaaata

SEQ ID NO: 123 - DNA, 21 bp, Artificial sequence
  Artificial Sequence = Primer
catcagttgctagtgcgaatg

SEQ ID NO: 124 - DNA, 20 bp, Artificial sequence
  Artificial Sequence = Primer
cagcaaatgtcaaatacgtt

SEQ ID NO: 125 - DNA, 21 bp, Artificial sequence
  Artificial Sequence = Primer
cggagagtacgaccggcgctt

SEQ ID NO: 126 - DNA, 21 bp, Artificial sequence
  Artificial Sequence = Primer
gcacggctggctgctttcgtt

SEQ ID NO: 127 - DNA, 21 bp, Artificial sequence
  Artificial Sequence = Primer
gctgccattaatagcgcaact

SEQ ID NO: 128 - DNA, 21 bp, Artificial sequence
  Artificial Sequence = Primer
tattgttgttaccagccttgc

SEQ ID NO: 129 - DNA, 21 bp, Artificial sequence
  Artificial Sequence = Primer
gtaatgacggttaattctgtt

SEQ ID NO: 130 - DNA, 21 bp, Artificial sequence
  Artificial Sequence = Primer
gccgcatcaatagccttagaa

SEQ ID NO: 131 - DNA, 20 bp, Artificial sequence
  Artificial Sequence = Primer
cccataacggaacaactcat

SEQ ID NO: 132 - DNA, 22 bp, Artificial sequence
  Artificial Sequence = Primer
cagaatagaccaaacatctgca

SEQ ID NO: 133 - DNA, 21 bp, Artificial sequence
  Artificial Sequence = Primer
ggccactttcaatgttggtca

SEQ ID NO: 134 - DNA, 22 bp, Artificial sequence
  Artificial Sequence = Primer
cgactgcacctgttcctgatta

SEQ ID NO: 135 - DNA, 21 bp, Artificial sequence
  Artificial Sequence = Primer
tctgatatagtttatatgggt

SEQ ID NO: 136 - DNA, 21 bp, Artificial sequence
  Artificial Sequence = Primer
tcaaaccccactcttaattaa

SEQ ID NO: 137 - DNA, 21 bp, Artificial sequence
  Artificial Sequence = Primer
ttgcaaaagcaattttgcaac

SEQ ID NO: 138 - DNA, 21 bp, Artificial sequence
  Artificial Sequence = Primer
tgccgaacaatgttctctgca

SEQ ID NO: 139 - DNA, 21 bp, Artificial sequence
  Artificial Sequence = Primer
aattgttttaaaatctgttct

SEQ ID NO: 140 - DNA, 21 bp, Artificial sequence
  Artificial Sequence = Primer
tgagactgaaattacatttaa

SEQ ID NO: 141 - DNA, 21 bp, Artificial sequence
  Artificial Sequence = Primer
gaacaattcaaacagttcagt

SEQ ID NO: 142 - DNA, 21 bp, Artificial sequence
  Artificial Sequence = Primer
ttattcaaatcgcgcaatacc

SEQ ID NO: 143 - DNA, 20 bp, Artificial sequence
  Artificial Sequence = Primer
caaatacagtccgcgtacga

SEQ ID NO: 144 - DNA, 21 bp, Artificial sequence
  Artificial Sequence = Primer
ccattgttacctaaagagcgt

SEQ ID NO: 145 - DNA, 21 bp, Artificial sequence
  Artificial Sequence = Primer
ttggcagttacaggaatgcat

SEQ ID NO: 146 - DNA, 21 bp, Artificial sequence
  Artificial Sequence = Primer
aacagtgaaccatattttagt

SEQ ID NO: 147 - DNA, 20 bp, Artificial sequence
  Artificial Sequence = Primer
atgaggaacataatggcagg

SEQ ID NO: 148 - DNA, 20 bp, Artificial sequence
  Artificial Sequence = Primer
tctggtcaggtcgtcaatac

SEQ ID NO: 149 - DNA, 21 bp, Artificial sequence
  Artificial Sequence = Primer
ggtgatcgattattccgctga

SEQ ID NO: 150 - DNA, 21 bp, Artificial sequence
  Artificial Sequence = Primer
acgcctcatcggtcagcgcct

SEQ ID NO: 151 - DNA, 21 bp, Artificial sequence
  Artificial Sequence = Primer
tctgcacgtttaaaattattg

SEQ ID NO: 152 - DNA, 21 bp, Artificial sequence
  Artificial Sequence = Primer
gtttctccgtcagaatcaagc

SEQ ID NO: 153 - DNA, 21 bp, Artificial sequence
  Artificial Sequence = Primer
ctaccgtagcgggcgatggta

SEQ ID NO: 154 - DNA, 21 bp, Artificial sequence
  Artificial Sequence = Primer
cagcgcctgtccgtgttcggc

SEQ ID NO: 155 - DNA, 21 bp, Artificial sequence
  Artificial Sequence = Primer
ccctgaccttgggtgttgcga

SEQ ID NO: 156 - DNA, 20 bp, Artificial sequence
  Artificial Sequence = Primer
gtactgaactttaaaggtgg

SEQ ID NO: 157 - DNA, 20 bp, Artificial sequence
  Artificial Sequence = Primer
aagaaatcaatattatttat

SEQ ID NO: 158 - DNA, 18 bp, Artificial sequence
  Artificial Sequence = Primer
aatagcacccggtacaag

SEQ ID NO: 159 - DNA, 20 bp, Artificial sequence
  Artificial Sequence = Primer
gcgaaggaatttaccttaga

SEQ ID NO: 160 - DNA, 20 bp, Artificial sequence
  Artificial Sequence = Primer
cagctgtcacagtaacaaac

SEQ ID NO: 161 - DNA, 20 bp, Artificial sequence
  Artificial Sequence = Primer
cttgaacatatatctcaggg

SEQ ID NO: 162 - DNA, 21 bp, Artificial sequence
  Artificial Sequence = Primer
acaggagcagtttcagacagt

SEQ ID NO: 163 - DNA, 21 bp, Artificial sequence
  Artificial Sequence = Primer
ggtggagtatacaaaatataa

SEQ ID NO: 164 - DNA, 21 bp, Artificial sequence
  Artificial Sequence = Primer
atgacaggcattagttttaat

SEQ ID NO: 165 - DNA, 20 bp, Artificial sequence
  Artificial Sequence = Primer
ttctgttaatgcaatggcgg

SEQ ID NO: 166 - DNA, 21 bp, Artificial sequence
  Artificial Sequence = Primer
ttcagcaaatccggagcctga

SEQ ID NO: 167 - DNA, 20 bp, Artificial sequence
  Artificial Sequence = Primer
gaagaagatgtttatagcgg

SEQ ID NO: 168 - DNA, 21 bp, Artificial sequence
  Artificial Sequence = Primer
actgcaggtattagatatgat

SEQ ID NO: 169 - DNA, 22 bp, Artificial sequence
  Artificial Sequence = Primer
attggtgccggtgttactgctg

SEQ ID NO: 170 - DNA, 20 bp, Artificial sequence
  Artificial Sequence = Primer
ctcccatacctaaacgcaat

SEQ ID NO: 171 - DNA, 21 bp, Artificial sequence
  Artificial Sequence = Primer
attggtgttgccgtcaccgct

SEQ ID NO: 172 - DNA, 18 bp, Artificial sequence
  Artificial Sequence = Primer
acgccatgacatgggagg

SEQ ID NO: 173 - DNA, 21 bp, Artificial sequence
  Artificial Sequence = Primer
attggtgctggtgtaacgact

SEQ ID NO: 174 - DNA, 18 bp, Artificial sequence
  Artificial Sequence = Primer
attgcgtttaggtatggg

SEQ ID NO: 175 - DNA, 21 bp, Artificial sequence
  Artificial Sequence = Primer
ctactgttcccgagtagtgtg

SEQ ID NO: 176 - DNA, 21 bp, Artificial sequence
  Artificial Sequence = Primer
ggtgccgtccggaatcggcgt

SEQ ID NO: 177 - DNA, 70 bp, Artificial sequence
  Artificial Sequence = Probe
gatgccgacagcgtcgagcgcgacagtgctcagaattacgatcaggggtatgttgggtttcacgtctggc

SEQ ID NO: 178 - DNA, 70 bp, Artificial sequence
  Artificial Sequence = Probe
caaagtggttagcgatatcttccgaagcaataaattcacgtaataacgttggcaagactggcatgataag

SEQ ID NO: 179 - DNA, 70 bp, Artificial sequence
  Artificial Sequence = Probe
gactggcgatgctgtcggaatggacgatatcccgcaagaggcccggcagtaccggcataaccaagcctat

SEQ ID NO: 180 - DNA, 70 bp, Artificial sequence
  Artificial Sequence = Probe
caaacgcggcacccgccagggataacagcagcaccggtctgcgccccagcttatctgaccatctgcccag

SEQ ID NO: 181 - DNA, 70 bp, Artificial sequence
  Artificial Sequence = Probe
gttgaggctgcaacagctccagtcgcaccggtaataccagcaattaagcgtcccaaatacaacacccaca

SEQ ID NO: 182 - DNA, 70 bp, Artificial sequence
  Artificial Sequence = Probe
ttaataaagccggaaccaccggcatgattaatcccaaaccaatcgcatcaagcgcgacaacaatgagtgc

(19) United States
(12) Patent Application Publication
(10) Pub. No.: US 2006/0094034 A1
(43) Pub. Date: May 4, 2006

(54) Virulence and antibiotic resistance array and uses thereof

(76) Inventors: Roland Brousseau, Montreal (CA); Jason Dubois, Ottawa (CA); Tom Edge, Toronto (CA); Luke Masson, Dollard-des-Ormeaux (CA); Jack T. Trevors, Guelph (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/136,524

(22) Filed: May 25, 2005

Related U.S. Application Data
(63) Continuation-in-part of application No. 10/425,821, filed on Apr. 30, 2003.

(51) Int. Cl. C12Q 1/68; C12M 1/34

(52) U.S. Cl. 435/6; 435/287.2

(57) ABSTRACT

An array of nucleic acid probes is described for simultaneously identifying or characterizing a pathotype of a microorganism and detecting antibiotic resistance of said microorganism. Methods are also described for detecting the presence of a microorganism in a sample, as well as determining its pathotype and its antibiotic resistance, using the array.

### CROSS-REFERENCE TO RELATED APPLICATIONS

This application is a continuation-in-part application of application Ser. No. 10/425,821 filed Apr. 30, 2003, still pending and also claims priority on U.S. provisional application Ser. 60/753,850 filed May 25, 2004, still pending, the entire content of both prior application being hereby incorporated in their entirety.

### TECHNICAL FIELD

The invention relates to an array and uses thereof and particularly relates to an array for characterizing a microorganism by its virulence and antibiotic resistance, and uses thereof.

### BACKGROUND OF THE INVENTION

A variety of pathogenic microorganisms exist, which pose a continued health threat. An example is the bacterium Escherichia coli, which is commonly found in the environment as well as in the digestive tracts of common animal species including humans. Individual strains within Escherichia coli (E. coli) can vary in pathogenicity from innocuous to highly lethal, as evidenced by incidents of its contamination of drinking water and outbreaks of so-called hamburger disease. Pathogenic forms of Escherichia coli (E. coli) are a worldwide cause of urinary tract infections, intestinal infections as well as septicemia and nosocomial infections. It is important that medicine can intervene effectively. One of medicine's arms against the E. coli infections is the use of antibiotics. However, an increase of antibiotic resistance is observed among E. coli strains. There are well over one hundred genes known to be directly involved in determining the degree and type of antibiotic resistance of E. coli. There is currently no practical, cost-effective way to determine rapidly and simultaneously the presence or the absence of this large set of these antibiotic resistance genes within a given E. coli strain. The genetic methods like genome analysis with DNA chips provide key information for guiding antibiotic therapy. But the most important problem is that presently, no technical product is offered to rapidly and simultaneously detect many resistance genes and mutations in a single step.

The pathogenicity of a given E. coli depends on the presence or absence of virulence genes within its genome. These virulence genes are ideal targets for the determination of the pathogenicity potential of any given E. coli isolate.

For virulence, the presence of virulence genes and the pathogenic behavior (so-called pathotype) are established by various combinations of microbiological methods including bacterial culture, immunoassay, tissue culture methods, PCR and microscopic analysis of biopsy samples. The same comments about slowness and expense apply here as well.

The above methods have been used for detecting and identifying pathogenic E. coli. However, these approaches suffer from a variety of limitations, the most serious of which is related to the large variety of virulence factors distributed among the known pathotypes. Currently, there is no practical, cost-effective way to determine rapidly and simultaneously the presence or absence of this large set of these virulence genes within a given E. coli strain.

For antibiotic resistance, basic microbiology tests (disk diffusion, broth dilution, agar dilution, and gradient diffusion) are the principal approach to get the phenotype of resistance rapidly. The bacteria have to be isolated and cultured before testing. Detection of antibiotic resistance genes can be accomplished with Polymerase Chain Reaction (PCR) amplification of target DNA and amplicon confirmation by gel electrophoresis and by probe hybridization techniques. Detection of gene mutations associated with antimicrobial resistance can be possible with the use of PCR-RFLP analysis, PCR-SSCP analysis, PCR-CFLP analysis, PCR-RNA combined with RNase cleavage assay, PCR amplification combined with DNA sequencing or with microarray analysis. The majority of these assays are impossible to do in one step, so the procedures are slow, complex and expensive.

A major drawback of the basic microbiology tests is that they are slow and tests give information about the phenotype only. There are also problems with other tests used to detect antibiotic resistance genes. First, they lack sensitivity when only a few organisms are present in the sample or when inhibitors are also present. Second, different assays are required for each antimicrobial agent tested or gene tested. False-positive results may occur due to contamination of the test sample with extraneous nucleic acid or residual nucleic acid from prior samples. The general situation of the tests used to detect mutations associated with antimicrobial, resistance is that the assays are insensitive, complex, slow, costly and may require several steps. A similar situation prevails for virulence genes.

Some publications show that DNA microarrays have been used for the detection of mutation associated with antimicrobial resistance of Mycobacterium tuberculosis. There are also publications that note that microarrays have been used for the detection of two resistance genes of the non pathogenic yeast Saccharomyces cerevisiae, for the detection of one resistance gene of M. tuberculosis, but not for pathogens having a large number of antibiotic resistance and virulence genes such as E. coli strains.

The published procedures for antibiotic resistance gene analysis and for virulence gene analysis using DNA microarrays all suffer from significant drawbacks and cannot currently be considered practical or cost-effective.

It would therefore be desirable to have improved methods and materials for the detection of pathogenic microorganisms, such as bacteria (e.g. E. coli).

### SUMMARY OF THE INVENTION

The invention relates to a collection of probes, e.g. in an array format, and uses thereof.

According to one aspect of the invention there is provided an apparatus for the simultaneous detection in a pathogen or in a liquid sample containing an unknown pathogen, of a plurality of antibiotic resistance and virulence genes, comprising a microarray, DNA probes e.g. synthetic oligonucleotides complementary for a plurality of currently known antibiotic resistance genes and virulence genes for a pathogen e.g. E. coli having such a plurality of known antibiotic resistance genes and virulence genes, immobilized on the microarray.

According to another aspect of the invention, a method is provided for simultaneous detection of a plurality of antibiotic resistance and virulence genes in a given liquid culture or colony of pathogen for the presence of these resistance and virulence genes comprising;
a) providing an unknown pathogen or a liquid sample containing an unknown pathogen;
b) extracting DNA from the pathogen;
c) labeling the DNA e.g. with a fluorescent dye,
d) providing a microarray, including a plurality of DNA probes immobilized thereon comprising synthetic oligonucleotides specific/complementary for known antibiotic resistance genes and virulence genes, and
e) applying the labeled DNA to the microarray, whereby the labeled DNA will hybridize with a DNA probe complementary for antibiotic resistance genes or virulence genes matching its DNA sequence.

Accordingly, in one aspect, the invention provides an array comprising: a substrate and a plurality of nucleic acid probes, each of the probes being bound to the substrate at a discrete location; the plurality of probes comprising at least one probe for at least one antibiotic resistance gene of a species of a microorganism and at least another probe for at least one virulence gene of the species. In an embodiment, the array comprises at least 103 distinct nucleic acid probes. In embodiments, each of the probes are independently greater than or equal to 15, 20, 50 or 100 nucleotides in length. In an embodiment, the array comprises a subarray, wherein the subarray comprises the at least two probes at adjacent discrete locations on the substrate.

In an embodiment, the microorganism is a bacterium, in a further embodiment, of the family Enterobacteriaceae, in a further embodiment, the bacterium is E. coli.

In an embodiment, the virulence gene can be one that codes for a pathotype selected from the group consisting of: enterotoxigenic E. coli (ETEC); enteropathogenic E. coli (EPEC); enterohemorrhagic E. coli (EHEC); enteroaggregative E. coli (EAEC); enteroinvasive E. coli (EIEC); uropathogenic strains (UPEC); E. coli strains involved in neonatal meningitis (MENEC); E. coli strains involved in septicemia (SEPEC); cell-etaching E. coli (CDEC); and diffusely adherent E. coli (DAEC).

In an embodiment, the virulence gene encodes a polypeptide of a class of proteins selected from the group consisting of toxins, adhesion factors, secretory system proteins, capsule antigens, somatic antigens, flagellar antigens, invasins, autotransporter proteins, and aerobactin system proteins. In an embodiment, the virulence gene is selected from the group consisting of afaBC3, afaE5, afaE7, afaD8, aggA, aggC, aida, bfpA, bmaE, cdt1, cdt2, cdt3, cfaI, clpG, cnf1, cnf2, cs1, cs3, cs31a, cvaC, derb122, eae, eaf, east1, ehxA, espA group I, espA group II, espA group III, espB group I, espB group II, espB group III, espC, espP, etpD, F17A, F17G, F18, F4, F41, F5, F6, fimA group I, fimA group II, fimH, mC, focG, fyuA, hlyA, hlyC, ibe10, iha, invX, ipaC, iroN, irp1, irp2, iss, iucD, iufA, katP, kfiB, kpsMTII, kpsMTIII, 17095, leoA, IngA, It, neuC, nfaE, ompA, ompT, paa, papAH, papC, papEF, papG group I, papG group II, papG group III, pai, rtbO9, rfbO101, rfbO111, rfbE 0157, rfbE O157H7, rfc O4, rtx, sfaDE, sfaA, stah, stap, stb, stx1, stx2, stxA I, stxA II, stxB I, stx B II, stxB III, tir group I, tir group II, tir group III, traT, and tsh genes. In an embodiment, the above-noted probe comprises a nucleic acid sequence selected from the group consisting of SEQ ID NO:1 to SEQ ID NO:102, or a fragment thereof, or a sequence substantially identical thereto. In the present invention, complete identity of the probes with the DNA to be detected is not essential, as partial identity or homology for detecting hybridization of the probes with the DNA to be detected can be sufficient. One skilled in the art will appreciate that by varying the hybridization conditions and the percentage of homology, same results can be achieved, depending on the selectivity or sensitivity desired for the array.

In an embodiment, the substrate is selected from the group consisting of a porous support and a support having a non-porous surface. In embodiments the support is selected from the group consisting of a slide, chip, wafer, membrane, filter and sheet. In an embodiment, the slide comprises a coating capable of enhancing nucleic acid immobilization to the slide. In an embodiment, the probes are covalently attached to the substrate.

The invention further provides a method of detecting the presence of a microorganism in a sample, the method comprising: contacting the above-mentioned array with a sample nucleic acid of the sample; and detecting association of the sample nucleic acid to a probe on the array; wherein association of the sample nucleic acid with the probe is indicative that the sample comprises a microorganism from which the nucleic acid sequence of the probe is derived. In an embodiment, the sample nucleic acid comprises a label. In an embodiment, the label is a fluorescent dye (e.g. a cyanine, a fluorescein, a rhodamine and a polymethine dye derivative). In an embodiment, the method further comprises extracting the sample nucleic acid from the sample before contacting it with the array. In an embodiment, the sample nucleic acid is not amplified by PCR prior to contacting it with the array. In an embodiment, the method further comprises digesting the sample nucleic acid with a restriction enzyme to produce fragments of the sample nucleic acid prior to contacting with the array. In an embodiment, the fragments are of an average size of about 0.2 Kb to about 12 Kb. In an embodiment, the method further comprises labeling the sample nucleic acid prior to contacting it with the array. In an embodiment, the sample nucleic acid is selected from the group consisting of DNA and RNA.

In an embodiment, the above-mentioned sample is selected from the group consisting of environmental samples, biological samples and food. In an embodiment, the environmental samples are selected from the group consisting of water, air and soil. In an embodiment, the biological samples are selected from the group consisting of blood, urine, amniotic fluid, feces, tissues, cells, cell cultures and biological secretions, excretions and discharge.

In an embodiment, the method is further for determining a pathotype and an antibiotic resistance of a species of the microorganism, wherein the probes are for a pathotype and an antibiotic resistance of the species and wherein association of the sample nucleic acid with the probes is indicative that the microorganism is of the pathotype and is resistant to the antibiotic tested.

In an embodiment, the sample is a tissue, body fluid, secretion or excretion from a subject and the method is further for diagnosing an infection by the microorganism in the subject, wherein association of the nucleic acid with the probe is indicative that the subject is infected by the microorganism.

In an embodiment, the method is for diagnosing a condition related to infection by the microorganism in the subject, wherein the probe is for a pathotype of the species and wherein association of the sample nucleic acid with the probe is indicative that the microorganism is of the pathotype and is antibiotic resistant and that the subject suffers from a condition associated with the pathotype. In an embodiment, the condition is selected from the group consisting of: diarrhea, hemorrhagic colitis, hemolytic uremic syndrome, invasive intestinal infections, dysentery, urinary tract infections, neonatal meningitis and septicemia. In an embodiment, the subject is a mammal, in a further embodiment, a human.

The invention further provides a commercial package comprising the above-mentioned array together with instructions for: (a) detecting the presence of a microorganism in a sample; (b) determining the pathotype of a microorganism in a sample; (c) determining antibiotic resistance of a microorganism in a sample; (d) diagnosing an infection by a microorganism in a subject; (e) diagnosing a condition related to infection by a microorganism, in a subject; or (f) any combination of (a) to (e).

The invention further provides a use of the above-mentioned array for: (a) detecting the presence of a microorganism in a sample; (b) determining the pathotype of a microorganism in a sample; (c) determining antibiotic resistance of a microorganism in a sample; (d) diagnosing an infection by a microorganism in a subject; (e) diagnosing a condition related to infection by a microorganism, in a subject; or (f) any combination of (a) to (e).

The invention further provides a method of producing an array for phenotyping a microorganism in a sample by its pathotype and antibiotic resistance, the method comprising: providing a plurality of nucleic acid probes, the plurality of probes comprising at least one probe for at least one antibiotic resistance gene of a species of the microorganism and at least one other probe for at least one pathotype of the species; and applying each of the probes to a different discrete location of a substrate. In an embodiment, the method further comprises the step of cross-linking by exposure of the array to ultraviolet radiation. In an embodiment, the method further comprises heating the array subsequent to the cross-linking.

The invention further provides a method of producing an array for phenotyping a microorganism in a sample by its pathotype and antibiotic resistance, the method comprising: selecting a plurality of nucleic acid probes, the plurality of probes comprising at least one probe for a first pathotype of a species of the microorganism and at least another one probe for detecting an antibiotic resistance gene of the species; and synthesizing or immobilizing each of the plurality of probes at a different discrete location of a substrate.

The invention combines the parallel processing power inherent in DNA microarrays with a very effective and robust labeling methodology, plus an optimized design of immobilized DNA probes to achieve practicality, robustness and cost effectiveness. Such a combination has not, to the inventors' knowledge, been reported in either the patent or scientific literature.

With regard to antimicrobial resistance, there are several reasons to pursue the identification of antibiotic resistance genes or mutations associated with antibiotic resistance in pathogens with DNA microarrays. First, DNA microarrays are helpful for arbitrating results which come from regular microbiology tests that are at or near the breakpoint for resistance for pathogenic species. Second, DNA microarrays can be used to detect resistance genes or mutations that result in resistance in organisms directly in clinical specimens to guide therapy early in the course of a patient's disease long before culture are positive. Third, DNA microarrays are more accurate than antibiograms for following the epidemiologic spread of a particular resistance gene in a hospital or a community setting.

The lower cost, higher reliability and increased flexibility of the new approach described herein, together with the combination of virulence and antibiotic resistance gene probes on the same array, amount to a breakthrough in usability and practicality.

### BRIEF DESCRIPTION OF THE DRAWINGS

FIG. 1: Print pattern of the E. coli pathotype microarray according to an embodiment of the invention. (A) Grouping of genes by category (B) Location of the individual genes.

FIG. 2: Print pattern of the virulence and antibiotic resistance 70-mer oligonucleotide microarray according to another imbodiment of the invention.

FIG. 3: Detection of virulence genes and simultaneous identification of the pathotype of known E. coli strains after microarray hybridization with genomic DNA from (A) a nonpathogenic K-12 E. coli strain DH5α (B) an enterohemorrhagic strain EDL933 O157:H7 (C) an uropathogenic strain J96, O4:K6 and (D) an enterotoxigenic strain H-10407. Genomic DNA after HindIII/EcoRI digestion was labeled with Cy3. Labeled DNA (500 ng) was hybridized to the array overnight at 42° C., washed, dried and scanned. Boxed spots in Panel A represent the virulence genes present in K-12 E. coli strain DH5α (traT, fimA, fimH, ompA, ompT, iss, fliC). Boxed spots in Panels B, C and D indicate the pathotype-specific genes in the tested strains. Genes present in more than one pathotype (iss, irp2, fliC, ompT) or present in all the pathotypes (fimH, fimA, ompA) gave a positive signal. The horizontal bar indicates the color representation of fluorescent-signal intensity.

FIG. 4: Virulence potential analysis of E. coli strains isolated from clinical samples using a E. coli pathotype microarray according to an embodiment of the invention. (A) Hybridization of genomic DNA from an avian E. coli isolate Av01-4156 (B) Hybridization pattern obtained with genomic DNA from a bovine strain B00-4830 (C) Hybridization of genomic DNA from a human E. coli isolate H87-540. Labeled DNA (500 ng) was hybridized to the array overnight at 42° C. after which the slide was washed, dried and scanned. Boxed spots indicate the pathotype-specific genes: iucD, iron, traT and iutA in panel A, etpD, F5, stap, and traT in panel B, stx1, cdt2, cdt3, afaD8, bmaE, iucD, iroN, and iutA in Panel C. Positive signals were also obtained with genes present in more than one pathotype (espP, iss, ompT, fliC) and genes present in all the tested pathotypes (fimA, fimH, ompA).

FIG. 5: Hybridization results obtained for the EHEC reference strain EDL933. Unexpected results are indicated by the rectangles: low fluorescence intensity was observed for the wzy(O157:H7) oligonucleotide, no signal was obtained for the eae(γ) oligonucleotide, and a false positive signal was obtained with the bfpA oligonucleotide.

FIG. 6: Detection of stx and cnf variant genes in clinical isolates of E. coli using a pathotype microarray according to an embodiment of the invention. The white boxes in Panel A outlines the stx genes hybridized with (1) the human strain H87-5406 and (2) the bovine strain B994297. The white boxes in Panel B outlines the cnf genes hybridized with (1) strain CaO1-E179 and (2) strain H87-5406. Labeled DNA (500 ng) was hybridized to an array overnight at 42° C. after which the slide was washed, dried and scanned.

FIG. 7: Use of an E. coli pathotype microarray according to an embodiment of the invention to identify the phylogenetic group of E. coli strains on the basis of their hybridization pattern with the attaching and effacing gene probes (A) print pattern of espA, espB and tir probes on the pathotype microarray with the homology percentages between each immobilized probe (B) detection of espA3, espB2 and tir3 in the human EPEC strain E2348/69 (C) hybridization pattern obtained with genomic DNA from the animal EPEC strain P86-1390 (espA1, espB3 and tir1 (D) detection of espA2, espB1 and tir2 in the EHEC strain EDL933. The positive hybridization results obtained with espa, espB and tir probes are outlined in white boxes.

FIG. 8: Coding key (8A) for the antibiotic resistance gene microarray and results obtained with such microarray (8B) on terminal transferase test.

FIG. 9: Results from hybridization of ETEC 353 with the antibiotic resistance microarray of the invention. The coding key is the same as in FIG. 8B.

FIG. 10: Results in the form of a comparison between two multiresistant Escherichia coil enterotoxigenic strains (ETEC 329 and ETEC 399) are illustrated, compared to a negative control E. coli which does not have antibiotic resistance genes.

FIG. 11: Results showing that the present invention can distinguish the single base pair mutant involved in mutation S83L, involved in fluoroquinolone resistance in E. coli, using the hybridization strategy described herein.

FIG. 12: Hybridization results obtained for the ExPEC strain 01-8344-0611 (isolated from an animal with septicemia) for the antibiotic resistance genes. Expected results are indicated by green rectangles. The red rectangle indicates the negative result obtained for tet(C), confirming the absence of cross-hybridization between tet(A) and tet(C) oligonucleotides.

### DETAILED DESCRIPTION OF THE PREFERRED EMBODIMENT

The method used for fabricating microarrays (except for the material affixed to the microarrays) is substantially that described by U.S. Pat. No. 6,110,426, the disclosure of which is incorporated herein by reference.

The basic concept of the DNA microarray as applied to antimicrobial resistance and virulence genes detection is as following. A bacterial sample which may come from environment, food, water, clinical sample from human or animal source is either incubated on a solid medium or in a liquid medium for culturing and multiplicating the microorganism that may be contained therein or is used directly with PCR techniques to amplify any DNA from microorganisms that may be present therein. When microorganisms are grown first, DNA is then extracted and labeled with a detectable marker, such as a fluorescent dye. If the DNA has been amplified by PCR directly, the amplified DNA is then labeled with the detectable label. The DNA labeled with the detectable label is then applied to an antibiotic resistance and virulence gene, DNA microarray. The fluorescent DNA will stick (by hybridization) wherever a complementary probe for antibiotic resistance or virulence gene matches its DNA sequence. Since the order and position of the probes is precisely determined, the content of antibiotic resistance genes and virulence genes in the initial sample is fully determined.

The present invention provides products and methods for the detection and characterization of microorganisms, such as bacteria, (e.g. of the family Enterobacteriaceae) such as E. coli. The products and methods of the invention can be used to detect the presence of such a microorganism in a sample (e.g. a biological or environmental sample). Further, such products and methods can be used to characterize such a microorganism, e.g. determining/characterizing its pathotype (virulence) and antibiotic resistance.

Pathogenic E. coli are responsible for three main types of clinical infections (a) enteric/diarrheal disease (b) urinary tract infections and (c) sepsis/meningitis. On the basis of their distinct virulence properties and clinical symptoms of the host, pathogenic E. coli are divided into numerous categories or pathotypes. The diarrheagenic E. coli include (i) enterotoxigenic E. coli (ETEC) associated with traveller's diarrhea and porcine and bovine diarrhea, (ii) enteropathogenic E. coli (EPEC) causing diarrhea in children and animals, (iii) enterohemorrhagic E. coli (EHEC) associated with hemorrhagic colitis and hemolytic uremic syndrome in humans, (iv) enteroaggregative E. coli (EAEC) associated with persistent diarrhea in humans, and (v) enteroinvasive E. coli (EIEC) involved in invasive intestinal infections, watery diarrhea and dysentery in humans and animals (Nataro, J. P., et al. (1998) Clin Microbiol Rev. 11:142-201). Extra-intestinal infections are caused by three separate E. coli pathotypes (i) uropathogenic strains (UPEC) that cause urinary tract infections in humans, dogs and cats (Beutin, L. (1999) Vet Res. 30:285-298; Garcia, E., et al. (1988) Antonie Van Leeuwenhoek. 54:149-163; and Wilfert, C. M. (1978) Annu Rev Med. 29:129-136) (ii) strains involved in neonatal meningitis (MENEC) (Wilfert, C. M. (1978) Annu Rev Med. 29:129-136) and (iii) strains that cause septicemia in humans and animals (SEPEC) (Dozois, C. M., et al. (1997) FEMS Microbiol Lett. 152:307-312; Harel, J., et al. (1993) Vet Microbiol. 38:139-155; Martin, C., et al. (1997) Res Microbiol. 148:55-64; and Wilfert, C. M. (1978) Annu Rev Med. 29:129-136).

Numerous bioassays and molecular methods have been developed for the detection of genes involved in pathogenic E. coli virulence mechanisms. However, the sheer numbers of known virulence factors have made this a daunting task. As described herein, microarray technology offers the most rapid and practical tool to detect the presence or absence of a large set of virulence genes simultaneously within a given E. coli strain. Prior to applicants' findings herein, only a few studies have reported the use of microarrays as a diagnostic tool (Call, D. R., et al. (2001) Int J Food Microbiol. 67:71-80; Chizhikov, V., et al. (2001) Appl Environ Microbiol. 67:3258-3263; Cho, J. C., et al. (2001) Appl Environ Microbiol. 67:3677-3682; Li, J., et al. (2001) J Clin Microbiol. 39:696-704; and Murray, A. E., et al. (2001) Proc Natl Acad Sci USA. 98:9853-9858). Described herein is a new approach for detection of a large number of virulence and antibiotic resistance factors present in E. coli strains and the subsequent determination of the strain's pathotype and antibiotic resistance. As described herein, nucleic acid sequences derived from most known virulence and antibiotic factors including associated-virulence genes and antibiotic resistance genes were amplified by PCR and immobilized onto glass slides to create a virulence and antibiotic resistance DNA microarray chip. Probing this virulence/antibiotic resistance gene microarray with labeled genomic E. coli DNA, the virulence and antibiotic resistance patterns of a given strain can be assessed and its pathotype determined in a single experiment.

As a practical example in support of this invention, an E. coli virulence and antibiotic resistance factor microarray was designed and tested. It was of course recognized that applications of this microarray reach far into human health, drinking water and environmental research.

According to another aspect of the invention, a method is provided for analyzing a given liquid culture or colony of bacteria simultaneously for the presence of a number of these virulence and antibiotic resistance genes in the same experiment.

In one embodiment, an array of virulence and antibiotic resistance genes may be used by reference laboratories involved in public or veterinary health. A simplified format of the microarray focusing on a few key virulence and antibiotic resistance genes could find a broader market in routine medical or veterinary microbiological laboratory work.

Other types of virulence and antibiotic resistance genes may be represented on such an array for a variety of applications. For example, the armed forces may be interested in implementing this type technology for detection and/or identification of biological warfare agents.

The invention thus relates to products and methods which enable the parallel analysis in respect of a plurality of pathotypes of a microorganism, and possibly of various antibiotic resistance, via the use of a collection of a plurality of nucleic acid probes derived from virulence and antibiotic resistance genes of the microorganism, the collection corresponding to a plurality of pathotypes and antibiotic resistance patterns of the microorganism. In an embodiment, the plurality of pathotypes may comprise at least 2, 3, 4, 5, 6, 7, 8, 9 or 10 pathotypes. In an embodiment, the plurality of antibiotic resistance patterns may comprise at least 2, 3, 4, 5, 6, 7, 8, 9 or 10 antibiotic resistance genes.

Accordingly, in an aspect, the invention relates to a collection comprising a plurality of probes, the probes being derived from genetic/protein (e.g. a virulence and antibiotic resistance genes) material/information from a microorganism and correspond to a plurality of pathotypes and antibiotic resistance patterns of the microorganism. In an embodiment, the probes comprise a nucleic acid sequence derived from a microorganism or a sequence substantially identical thereto. In an embodiment, the collection can represent more than one microorganism.

“Pathotype” as used herein refers to the classification of a particular strain of a microorganism by virtue of the pathogenic phenotype it may manifest when it infects a subject. A plurality of strains may thus be grouped in the same pathotype if the strains are capable of resulting in the same phenotypic manifestation (e.g. disease symptoms) when they infect a subject. In the case of E. coli, for example, pathotypes may include those associated with intestinal and extraintestinal conditions. Such pathotypes include but are not limited to ETEC, EPEC, EHEC, EAEC, EIEC, UPEC, MENEC, SEPEC, CDEC and DAEC noted herein. As described herein, a pathotype may be identified and/or characterized using a probe based on a virulence gene associated with the pathotype, in a particular microorganism (See Table 1).

TABLE 1
Pathotype grouping of E. coli virulence genes

| Pathotype | Pathotype-specific virulence genes |
|---|---|
| UPEC | sfaA; sfaDE; clpG; iutA; nfaE; pai; iroN; cvaC; kpsMT2; |
| | kpsMT3; hlyA; hlyC; focG; afaD8; bmaE; cs31A; |
| | drb122; kfiB; afa3; afa5; afaE7; papEF; papC; papGI; |
| | papGII; papGII; papAH |
| ETEC | IngA; sth; stp; stb; It; F18; F41; leoA; rfbO101; F5; F6; |
| | F17A; F17G; cfaI; cs1; cs3; F4 |
| EPEC | bfpA; eaf; espC |
| EHEC | ehxA; etpD; katP; L9075; rfbEO157; rfbO111; |
| | rfbO157H7; rtx; stx1; stx2; stxA1; stxA2;; StxB1; StxB2; |
| | Stx3A |
| EPEC and | eae; espP; espA1; espA2; espA3; paa; espB1; espB2; |
| EHEC | espB3; tir1; tir2; tir3; espC |
| (i.e. common | |
| to both) | |
| DAEC | aida |
| EAEC | aggA; aggC |
| EIEC | ipaC; invX |
| CDEC | cdt1; cdt2; cdt3; cnf1; cnf2 |
| MENEC | rfcO4; iucD; ibe10; neuC; rfbO9 |

“Virulence gene” as used herein refers to a nucleic acid sequence of a microorganism, the presence and/or expression of which correlates with the pathogenicity of the microorganism. In the case of bacteria, such virulence genes may in an embodiment comprise chromosomal genes (i.e. derived from a bacterial chromosome), or in a further embodiment comprise a non-chromosomal gene (i.e. derived from a bacterial non-chromosomal nucleic acid source, such as a plasmid). In the case of E. coli, examples of virulence genes and classes of polypeptides encoded by such genes are described below. Virulence genes for a variety of pathogenic microorganisms are known in the art.

The term probe as used herein is intended to mean any fragment of nucleic acid sufficient to hybridize with a target nucleic acid (generally DNA) to be detected. The fragment can vary in length from 15 nucleotides up to hundreds or thousands of nucleotides. Determination of the length of the fragment is a question of the desired sensitivity, of cost and/or the specific conditions used in the assay.

In an embodiment, the above-noted collection is in the form of an array, whereby the probes are bound to different, discrete locations of a substrate. The length of the probes may be variable, e.g. at least 15, 20, 50, 100, 500, 1000 or 2000 nucleotides in length. High density nucleic acid probe arrays, also referred to as “microarrays,” may for example be used to detect and/or monitor the expression of a large number of genes, or for detecting sequence variations, mutations and polymorphisms. Microfabricated arrays of large number of oligonucleotide probes, (variously described as “biological chips”, “gene chips”, or “DNA chips”), allow the simultaneous nucleic acid hybridization analysis of a target DNA molecule with a very large number of oligonucleotide probes. In one aspect, the invention provides biological assays using such high density nucleic acid or protein probe arrays. For the purpose of such arrays, “nucleic acids” may include any polymer or oligomer of nucleosides or nucleotides (polynucleotides or oligonucleotides), which include pyrimidine and purine bases, preferably cytosine, thymine, and uracil, and adenine and guanine, respectively. Polymers or oligomers of deoxyribonucleotides or ribonucleotides may be used, which may contain naturally occurring or modified bases, and which may contain normal internucleotide bonds or modified (e.g. peptide) bonds. A variety of methods are known for making and using microarrays, as for example disclosed in Cheung, V. G. et al. (1999) Nature Genetics Supplement, 21, 15-19; Lipshutz, R. J. et al., (1999) Nature Genetics Supplement, 21, 20-24; Bowtell, D. D. L. (1999) Nature Genetics Supplement, 21, 25-32; Singh-Gasson, S. et al. (1999) Nature Biotechnol. 17, 974-978; and, Schweitzer, B. et al. (2002) Nature Biotechnol. 20, 359-365; all of which are incorporated herein by reference. DNA chip technology is described in detail in, for instance, U.S. Pat. No. 6,045,996 to Cronin et al., U.S. Pat. No. 5,858,659 to Sapoisky et al., U.S. Pat. No. 5,843,655 to McGall et al., U.S. Pat. No. 5,837,832 to Chee et al., and U.S. Pat. No. 6,110,426 to Shalon et al., all of which are specifically incorporated herein by reference. Suitable DNA chips are available for example from Affymetrix, Inc. (Santa Clara, Calif.).

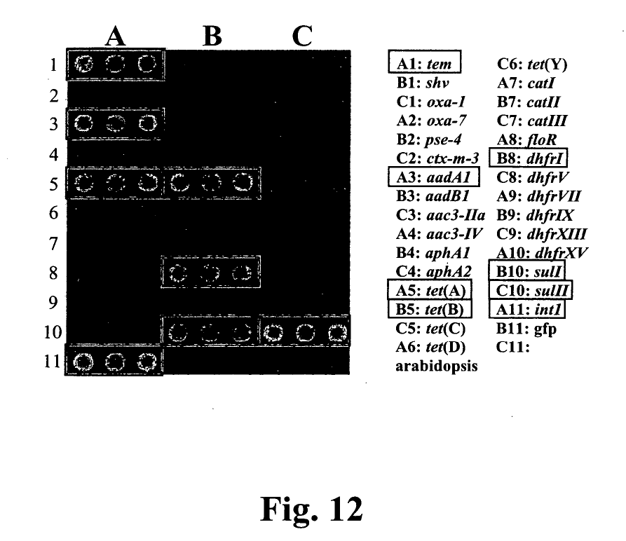

In another embodiment, a 70-mer oligonucleotide microarray was developed in order to determine simultaneously the presence or absence of a large set of virulence and antimicrobial resistance genes withinm including closely-related variants, within a given E. coli isolate. This embodiment contains oligonucleotides designed from the previous virulence midroarray, oligonucleotides specific for antimicrobial resistance genes previously characterized in various E. coli strains, and oligonucleotides specific for new putative virulence genes described in E. coli. 70-mer oligonucleotides were preferred to amplicons on the basis of earlier results obtained with amplicon-based microarrays, which found that amplicon probes had a high potential to cross-hybridize while oligonucleotide probes were more specific. Indeed, contrary to amplicon-based microarray and other molecular methods, such as membrane hybridizations, no cross-hybridization was observed between genes showing a high percentage of identity in their nucleic sequences. As an example, the absence of cross-hybridization, confirmed by PCR, between tetC and tetA genes, which show more than 75 percent of identity in their nucleic sequence, features the 70-mer oligonucleotide microarray specificity (see FIG. 12). In addition, 70-mer oligonucleotides also improved specificity by allowing the discrimination of variants of a single gene which show less than 10 percent divergence in their nucleic sequences, while amplicons did not.

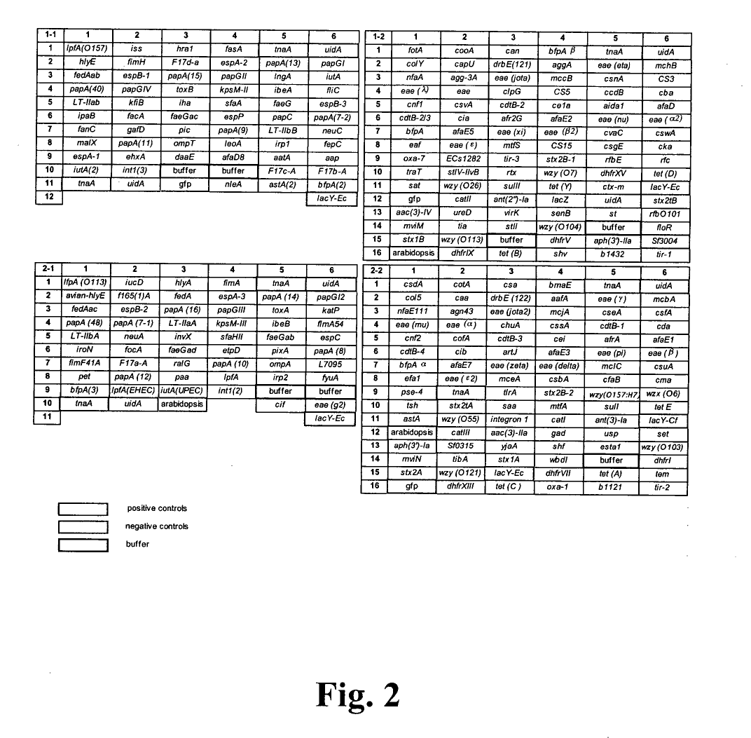

Two hundred and ninety one 70-mer oligonucleotides were designed for the elaboration of the virulence and antibiotic resistance array (see Table 7). Thirty three of them correspond to 30 antimicrobial resistance genes characteristically found in E. coli strains and to the class 1 integron. Because of one false positive result obtained with the first oligonucleotide specific for class 1 integron, I have designed 2 new 70-mer oligonucleotides. These two ones, int1(2) and int1(3), were respectively specific for the conserved region (qacEdelta1) and for the integrase gene of the class 1 integron. The 258 other oligonucleotides were designed either from the previous virulence amplicon-based microarray or correspond to new putative virulence genes recently described in E. coli strains. Among them, four were specific for bacterial species (lacY-Ec for E. coli, lacY-Cf for Citrobacter freundii, Sf0315 and Sf3004 for Shigella flexnen), three were positive controls (lacZ, uldA and tnaA), and two were negative controls (gfp and Arabidopsis thaliana) (FIG. 2). The 249 remaining oligonucleotides were specific for virulence genes (encoding toxins, hemolysins, fimbrial and afimbrial adhesins, cytotoxic factors . . . ) and virulence-associated genes (microcins and colicins).

For antimicrobial resistance genes and virulence genes from the previous virulence microarray, oligonucleotides were designed either from published PCR primers which were lengthened to 70 bases, or designed using the software program “OligoPicker”(Wang and Seed, 2003). For all of the new virulence genes or associated-virulence genes, the (public domain) “OligoPicker” software was used to design oligonucleotides. When different variants were found for a single gene, multiple alignments and phylogenetic analysis were performed to identify variant-specific probes. When 10% of divergence or more was observed between the DNA sequence of two variants, one oligonucleotide was designed for each one. Compared to the previous virulence amplicon-based microarray, this particular embodiment adds 59 oligonucleotides specific for fimbrial or afimbrial adhesins genes (30) or gene variants (29), 13 oligonucleotides specific for colicin genes and 7 oligonucleotides specific for microcins, 18 oligonucleotides specific for the different eae (intimine) gene variants, 8 oligonucleotides specific for toxins genes or gene variants, 29 oligonucleotides specific for various virulence genes or gene variants recently described in E. coli, and 6 oligonucleotides specific for putative new virulence genes.

As shown in FIG. 2, the microarray is composed by four subarrays and contains the 291 70-mer oligonucledtides which were printed in triplicates on Corning Ultra GAPS slides. In order to facilitate hybridization analysis, each subarray contains two positive controls in the right upper corner. For statistical analysis and to avoid problem of local background, positive and negative controls as well as buffer were dispatched inside all of the four subarrays (FIG. 2).

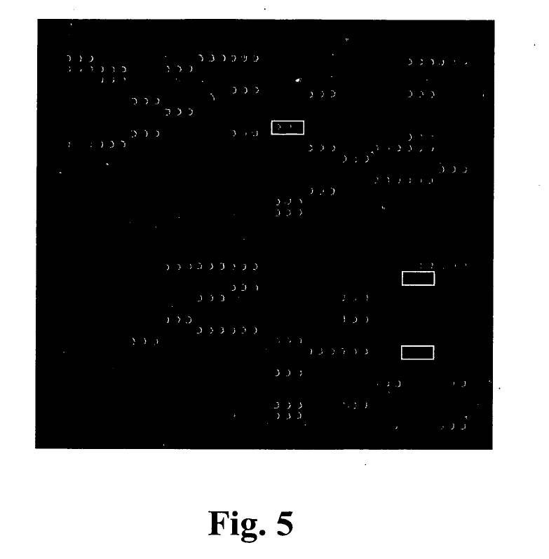

Validation of the oligonucleotide microarray took advantage of the availability of full genome sequences from thee references together with our large collection of characterized E. coli isolates. DNA from the three E. coli reference strains EDL933 (EHEC), CFT073 (UPEC) and MG1655 (K12), and from a collection of 20 well-characterized E. coli isolates (strains characterized with the previous virulence amplicon-based microarray or by membrane hybridizations) was hybridized to the oligonucleotide microarray. Hybridizations with these known labeled genomic DNA validated our microarray as a powerful tool for the detection of virulence and antimicrobial resistance genes in E. coli isolates. As shown in FIG. 5, only a few unexpected results were obtained for all of the strains tested. The false positive results were corrected by adding other oligonucleotides specific for the targeted gene, and the false negative results were corrected by adding oligonucleotides designed from sequences of other variants from the targeted genes.

Methods for storing, querying and analyzing microarray data have for example been disclosed in, for example, U.S. Pat. No. 6,484,183 issued to Balaban, et al. Nov. 19, 2002; and U.S. Pat. No. 6,188,783 issued to Balaban, et al. Feb. 13, 2001; Holloway, A. J. et al., (2002) Nature Genetics Supplement, 32, 481-489; each of which is incorporated herein by reference.

DNA chips generally include a solid substrate or support, and an array of oligonucleotide probes immobilized on the substrate. The substrate can be, for example, silicon or glass, and can have the thickness of a glass microscope slide or a glass cover slip. Substrates that are transparent to light are useful when the method of performing an assay on the chip involves optical detection. Suitable substrates include a slide, chip, wafer, membrane, filter, sheet and bead. The substrate can be porous or have a non-porous surface. Preferably, oligonucleotides are arrayed on the substrate in addressable rows and columns. A “subarray” may thus be designed which comprises a particular grouping of probes at a particular area of the array, the probes immobilized at adjacent locations or within a defined region of the array. A hybridization assay is performed to determine whether a target DNA molecule has a sequence that is complementary to one or more of the probes immobilized on the substrate. Because hybridization between two nucleic acids is a function of their sequences, analysis of the pattern of hybridization provides information about the sequence of the target molecule. DNA chips are useful for discriminating variants that may differ in sequence by as few as one or a few nucleotides.

Hybridization assays on the DNA chip involve a hybridization step and a detection step. In the hybridization step, a hybridization mixture containing the labeled target nucleic acid sequence is brought into contact with the probes of the array and incubated at a temperature and for a time appropriate to allow hybridization between the target and any complementary probes. The array may optionally be washed with a wash mixture which does not contain the target (e.g. hybridization buffer) to remove unbound target molecules, leaving only bound target molecules. In the detection step, the probes to which the target has hybridized are identified. Since the nucleotide sequence of the probes at each feature is known, identifying the locations at which target has bound provides information about the particular sequences of these probes.

Hybridization may be carried out under various conditions depending on the circumstances and the level of stringency desired. Such factors shall depend on the specificity and degree of differentiation between target sequences for any given analysis. For example, to distinguish target sequences which differ by only one or a few nucleotides, conditions of higher stringency are generally desirable. Stringency may be controlled by factors such as the content of hybridization and wash solutions, the temperature of hybridization and wash steps, the number and duration of hybridization and wash steps, and any combinations thereof. In embodiments, the hybridization may be conducted at temperatures ranging from about 4° C. up to about 80° C., depending on the length of the probes, their G+C content and the degree of divergence to be detected. If desired, denaturing reagents such as formamide may used to decrease the hybridization temperature at which perfect matches will dissociate. Commonly used conditions involve the use of buffers containing about 30% to about 50% formamide at temperatures ranging from about 20° C. to about 50° C. An example of such a partially denaturing buffer which is commercially available is the DIG Easy Hyb™ (Roche) buffer. In embodiments, un-labelled nucleic acids such as transfer RNA (tRNA) and salmon sperm DNA may be added to the hybridization buffers to reduce background noise. Under certain conditions, a divergence of 15% over long fragments (greater than 50 bases) can be reliably detected. Single nucleotide mistmatches in shorter fragments (15 to 25 nucleotides in length) can be also detected if the hybridization conditions are designed accordingly. Hybridization time typically ranges from about one hour to overnight (16 to 18 hours approximately). After hybridization, microarrays are typically washed one to five times in buffered salt solutions such as saline-sodium citrate, abbreviated SSC, for periods of time and at salt concentrations and temperature appropriate for a particular objective. A representative procedure may for example comprise three washes in pre-warmed (50° C.) 0.1×SSC (1×SSC contains 150 mM NaCl and 15 mM trisodium citrate, pH 7). In embodiments, a detergent such as sodium dodecyl sulfate [SDS; e.g. at 0.1% (w/v)] may be added to the washing buffer. Various details of hybridization conditions, some of which are described herein, are known in the art.

Hybridization may be performed under absolute or differential formats. The former refers to hybridization of nucleic acids from one sample to an array, and the detection of the nucleic acids thus hybridized. The differential hybridization format refers to the application of two samples, labeled with different labels (e.g. Cy3 and Cy5 fluorophores), to the array. In this case differences and similarities between the two samples may be assessed.

Many steps in the use of the DNA chip can be automated through use of commercially available automated fluid handling systems. For instance, the chip can be manipulated by a robotic device which has been programmed to set appropriate reaction conditions, such as temperature, add reagents to the chip, incubate the chip for an appropriate time, remove unreacted material, wash the chip substrate, add reaction substrates as appropriate and perform detection assays. If desired, the chip can be appropriately packaged for use in an automated chip reader.

The target polynucleotide, whose sequence is to be determined is usually labeled at one or more nucleotides with a detectable label (e.g. detectable by spectroscopic, photochemical, biochemical, chemical, bioelectronic, immunochemical, electrical or optical means). The detectable label may be, for instance, a luminescent label. Useful luminescent labels include fluorescent labels, chemi-luminescent labels, bio-luminescent labels, and colorimetric labels, among others. Most preferably, the label is a fluorescent label such as a cyanine, a fluorescein, a rhodamine, a polymethine dye derivative, a phosphor, and so forth. Suitable fluorescent labels are described in for example Haugland, Richard P., 2002 (Handbook of Fluorescent Probes and Research Products, ninth edition, Molecular. Probes). The label may be a light scattering label, such as a metal colloid of gold, selenium or titanium oxide. Radioactive labels such as ³²P, ³³P or ³⁵S can also be used.

When the target strand is prepared in single-stranded form, the sense of the strand should be complementary to that of the probes on the chip. In an embodiment, the target is fragmented before application to the chip to reduce or eliminate the formation of secondary structures in the target. Fragmentation may be effected by mechanical, chemical or enzymatic means. The average size of target segments following fragmentation is usually larger than the size of probe on the chip.

In embodiments, the target or sample nucleic acid may be extracted from a sample or otherwise enriched prior to application to or contacting with the array. Samples may amplified by suitable methods, such as by culturing a sample in suitable media (e.g. Luria-Bertani media) under suitable culture conditions to effect growth of microorganisms in the sample. Extraction may be performed using methods known in the art, including various treatments such as lysis (e.g. using lysozyme), heating, detergent (e.g. SDS) treatment, solvent (e.g. phenol-chloroform) extraction, and precipitation/resuspension. In an embodiment, the nucleic acid is not amplified using polymerase chain reaction (PCR) methods prior to application to the array.

In an embodiment, the probes may be provided, for example as a suitable solution, and applied to different, discrete regions of the substrate. Such methods are sometimes referred to as “printing” or “pinning”, by virtue of the types of apparatus and methods used to apply the probe samples to the substrate. Suitable methods are described in for example U.S. Pat. No. 6,110,426 to Shalon et al. The probe samples may be prepared by a variety of methods, including but not limited to oligonucleotide synthesis, as a PCR product using specific primers, or as a fragment obtained by restriction endonuclease digestion of a nucleic acid sample. Interaction/binding of the probe to the substrate may be enforced by non-covalent interactions and covalent attachment, for example via charge-mediated interactions as well as attachment to the substrate via specific reactive groups, crosslinking and/or heating.

In an embodiment, the arrays may be produced by, for example, spatially directed oligonucleotide synthesis. Methods for spatially directed oligonucleotide synthesis include, without limitation, light-directed oligonucleotide synthesis, microlithography, application by ink jet, microchannel deposition to specific locations and sequestration with physical barriers. In general these methods involve generating active sites, usually by removing protective groups; and coupling to the active site a nucleotide which, itself, optionally has a protected active site if further nucleotide coupling is desired.

In embodiments, the probes can be bound to the substrate through a suitable linker group. Such groups may provide additional exposure to the probe. Such linkers are adapted to comprise a terminal portion capable of interacting or reacting with the substrate or groups attached thereto, and another terminal portion adapted to bind/attach to the probe molecule.

Samples of interest, e.g. samples suspected of comprising a microorganism, for analysis using the products and methods of the invention include for example environmental samples, biological samples and food. “Environmental sample” as used herein refers to any medium, material or surface of interest (e.g. water, air, soil). “Biological sample” as used herein refers to a sample obtained from an organism, including tissue, cells or fluid. Biological excretions and secretions (e.g. feces, urine, discharge) are also included within this definition. Such biological samples may be derived from a patient, such as an animal (e.g. vertebrate animal, humans, domestic animals, veterinary animals and animals typically used in research models). Biological samples may further include various biological cultures and solutions.

The probes utilized herein may in embodiments comprise a nucleotide sequence identical to a nucleic acid derived from a microorganism or substantially identical, homologous or orthologous to such a nucleic acid. “Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness as orthologous does). Two nucleic acid sequences are considered “substantially identical” if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25% identity, with a sequence of interest.

Substantially complementary nucleic acids are nucleic acids in which the “complement” of one molecule is substantially identical to the other molecule. Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nim.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (WV) of II, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

An alternative indication that two nucleic acid sequences are substantially complementary is that the two sequences hybridize to each other under moderately stringent, or preferably stringent, conditions. Hybridization to filter-bound sequences under moderately stringent conditions may, for example, be performed in 0.5 M NaHPO₄, 7% (w/v) sodium dodecyl sulfate (SDS), 1 mM EDTA at 65° C., and washing in 0.2×SSC/0.1% (w/v) SDS at 42° C. (see Ausubel, et al. (eds), 1989, Current Protocols in Molecular Biology, Vol. 1, Green Publishing Associates, Inc., and John Wiley & Sons, Inc., New York, at p. 2.10.3). Alternatively, hybridization to filter-bound sequences under stringent conditions may, for example, be performed in 0.5 M NaHPO₄, 7% (w/v) SDS, 1 mM EDTA at 65*C, and washing in 0.1×SSC/0.1% (w/v) SDS at 68° C. (see Ausubel, et al. (eds), 1989, supra). Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

The above pre-existing elements were combined for the first time into a unique combination that surpasses others in terms of defining a robust, straightforward, practical and above all useable procedure. No similar work exists in the literature to the inventors' knowledge.

The present invention fully solves the problem by using synthetic oligonucleotides as gene probes. Additionally, the juxtaposition of antibiotic resistance genes and virulence genes on the same microarray greatly increases the usefulness of the Invention by simultaneously providing two independent sets of very important data.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

The present invention will be more readily understood by referring to the following examples which are given to illustrate the invention rather than to limit its scope.

### EXAMPLE I

Strains and Media

E. coli strains used to produce PCR templates are listed in Table 2. E. coli isolates including characterized strains (the non-pathogenic K12-derived E. coli strain DH5α, the enterohemorrhagic strain EDL933, the uropathogenic strain J96, the enterotoxigenic strain H-10407 and the enteropathogenic strains E2348/69 and P86-1390) and uncharacterized clinical strains from bovine (B00-4830, B99-4297), avian (Av01-4156), canine (Ca01-E179) and human (H87-5406) origin were used to assess the detection thresholds and hybridization specificity of the virulence microarray. Most of the E. coli strains were obtained from the Escherichia coli laboratory collection at the Faculté de médecine vétérinaire of the Université de Montreal. E. coli strains A22, AL851, C248 were kindly provided by Carl Marrs (University of Michigan) and IA2 by J. R. Johnson (University of Minnesota) respectively. All strains were stored in Luria-Bertani broth (LB [6]) broth plus 25% (v/v) glycerol at −80° C. E. coli cultures were grown at 37° C. in LB broth for genomic DNA extraction and purification. Alternatively, the bacterial strains are kept as a culture collection at −80° C. in tryptic soy broth (TSB) medium containing 10% (v/v) glycerol. Two aliquots of each strain are simultaneously plated on tryptic soy agar (TSA) supplemented with 5% (v/v) sheep blood as a quality control (purity of the strains) and resuspended in 10 ml of LB broth. Cells are grown overnight at 37° C. An agitation of 250 rpm is required for the liquid cultures (LB broth).

TABLE 2
Genes targeted, primers sources and strains
used as PCR amplification templates

| | Accession | Size | SEQ | |
|---|---|---|---|---|
| Gene | number | (bp) | ID NO: | Strains |
| afaBC3 | X76688 | 793 | 1 | A22 |
| afaE5 | X91748 | 470 | 2 | AL 851 |
| afaE7 | AF072901 | 618 | 3 | 262-KH 89 |
| afad8 | AF072900 | 351 | 4 | 2787 |
| agga | U12894 | 432 | 5 | Strain 17.2 |
| aggc | U12894 | 528 | 6 | Strain 17.2 |
| aida | X65022 | 644 | 7 | 2787 |
| bfpa | U27184 | 324 | 8 | O126:H6 E2348/69 |
| bmae | M15677 | 505 | 9 | 215 |
| cdt1 | U03293 | 412 | 10 | O15:KRVC383 OvinS5 |
| cdt2 | U042208 | 556 | 11 | O15:KRVC383 OvinS5 |
| cdt3 | U89305 | 556 | 12 | O15:KRVC383 OvinS5 |
| cfai | S73191 | 479 | 13 | H-10407 cfaI |
| clpg | M55389 | 403 | 14 | 215 |
| cnf1 | X70670 | 1112 | 15 | J96 O4:K12 |
| cnf2 | U01097 | 1240 | 16 | O15:KRVC383 OvinS5 |
| cs1 | M58550 | 321 | 17 | PB-176P cfa−II |
| cs3 | M35657 | 401 | 18 | PB-176 cfa+ II |
| cs31a | M59905 | 710 | 19 | 31a |
| CvaC | X57525 | 680 | 20 | 1195 |
| derb122 | U87541 | 260 | 21 | O4:K12 J96 |
| eae | U66102 | 791 | 22 | O157:H7 STJ348 |
| eaf | X76137 | 397 | 23 | O126:H6 E2348/69 |
| east1 | L11241 | 117 | 24 | O149:K9 1P97-2554B |
| ehxa | AF043471 | 158 | 25 | O157:H7 STJ348 |
| espa group I | AF064683 | 478 | 26 | P86-1390 |
| espA group | AF071034 | 523 | 27 | O157:H7 EDL933 |
| II | | | | |
| espA group | AJ225016 | 481 | 28 | O126:H6 E2348/69 |
| III | | | | |
| espB group I | AF071034 | 502 | 29 | O157:H7 EDL933 |
| espB group | Z21555 | 377 | 30 | O126 H6 E2348/69 |
| II | | | | |
| espB group | X99670 | 395 | 31 | P86-1390 |
| III | | | | |
| espC | AF297061 | 500 | 32 | O126 H6 E2348/69 |
| espP | AF074613 | 1830 | 33 | 215 |
| etpD | Y09824 | 509 | 34 | O157:H7 EDL933 |
| F17A | AF022140 | 441 | 35 | O15:KRVC3B3 OvinS5 |
| F17G | L33969 | 950 | 36 | O15:KRVC383 OvinS5 |
| F18 | M61713 | 510 | 37 | O139:K82 P88-1199 |
| F4 | M29374 | 601 | 38 | O149:K91 P97-2554B |
| F41 | X14354 | 431 | 39 | O9:K30 B44s |
| F5 | M35282 | 450 | 40 | O9:K30 B44s |
| F6 | M35257 | 566 | 41 | O9:K-P81-603A |
| fimA group I | Z37500 | 331 | 42 | 3292 |
| fimA group | Z37500 | 331 | 42 | O157:H7 EDL933 |
| II | | | | |
| fimH | AJ225176 | 508 | 43 | O157:H7 EDL933 |
| fliC | U47614 | 625 | 44 | O157:H7 E32511 |
| focG | S68237 | 359 | 45 | O4:K12 J96 |
| fyuA | Z38064 | 207 | 46 | 1195 |
| hlyA | M10133 | 500 | 47 | O4:K12 J96 |
| hlyC | M10133 | 556 | 48 | O4:K12 J96 |
| ibe10 | AF289032 | 170 | 49 | O18 H87-5480 |
| iha | AF126104 | 827 | 50 | O157:H7 E32511 |
| invX | L18946 | 258 | 51 | H84 (EIEC) |
| ipaC | X60777 | 500 | 52 | O157:H7 E32511 |
| iroN | AF135597 | 668 | 53 | CP9 |
| irp1 | AF091251 | 1689 | 54 | 1195 |
| irp2 | L18881 | 1241 | 55 | 1195 |
| iss | X52665 | 607 | 56 | 3292 |
| iucD | M18968 | 778 | 57 | 4787 |
| iutA | X05874 | 300 | 58 | 4787 |
| katP | X89017 | 2125 | 59 | O157:H7 EDL933 |
| kfiB | X77617 | 501 | 60 | K5(F9) 3669 |
| KpsMTII | X53819 | 270 | 61 | K5(F9) 3669 |
| KpsMTIII | AF007777 | 390 | 62 | 215 |
| I7095 | AF074613 | 659 | 63 | O157:H7 EDL933 |
| leoA | AF170971 | 501 | 64 | O149:K91 P97-2554B |
| IngA | AF004308 | 424 | 65 | PB-176P cfa−II |
| It | J01646 | 275 | 66 | O149:K91 P97-2554B |
| neuC | M84026 | 500 | 67 | O2:K1 U9/41 |
| nfaE | S61970 | 537 | 68 | 31a |
| ompA | V00307 | 1422 | 69 | O4:K12 J96 |
| ompT | X06903 | 559 | 70 | O4:K12 J96 |
| paa | U82533 | 360 | 71 | O157:H7 STJ348 |
| papAH | X61239 | 721 | 72 | O4:K12 J96 |
| papC | X61239 | 318 | 73 | 4787 |
| papEF | X61239 | 336 | 74 | O4:K12 J96 |
| PapG group I | M20146 | 461 | 75 | O4:K12 J96 |
| PapG group | M20181 | 190 | 76 | IA2 |
| II | | | | |
| PapG group | X61238 | 268 | 77 | O4:K12 J96 |
| III | | | | |
| pai | AF081286 | 922 | 78 | h140 8550 |
| rfbO9 | D43637 | 501 | 79 | O9:F6 K P81-603A |
| RfbO101 | X59852 | 500 | 80 | O101 h510a |
| RfbO111 | AF078736 | 406 | 81 | O111 H87-5457 |
| RfbE O157 | S83460 | 292 | 82 | O157:H7 EDL933 |
| RfbE O157 | S83460 | 259 | 83 | O157:H7 STJ348 |
| H7 | | | | |
| Rfc O4 | U39042 | 786 | 84 | O4:K12 J96 |
| rtx | AE005229 | 521 | 85 | O157:H7 EDL933 |
| sfaDE | X16664 | 408 | 86 | 4787 |
| sfaA | X16664 | 500 | 87 | 4787 |
| stah | M29255 | 201 | 88 | H-10407 |
| stap | M58746 | 163 | 89 | O149:K91 P97-2554B |
| stb | M35586 | 368 | 90 | O149:K91 P97-2554B |
| stx1 | L04539 | 583 | 91 | O157:H7 EDL933 |
| stx2 | AF175707 | 779 | 92 | O157 KNIH317 |
| stxA I | M23980 | 502 | 93 | O157:H7 EDL933 |
| stxA II | Y10775 | 482 | 94 | O157:H7 EDL933 |
| stxB I | M23980 | 151 | 95 | O157:H7 EDL933 |
| stx B II | Y10775 | 211 | 96 | O157:H7 EDL933 |
| stxB III | M36727 | 226 | 97 | O101 h510a |
| tir group I | AF045568 | 442 | 98 | RDEC-1B |
| tir group II | AF070067 | 479 | 99 | O157:H7 EDL933 |
| tir group III | AB036053 | 443 | 100 | O126:H6 E2348/69 |
| traT | J01769 | 288 | 101 | 3292 |
| tsh | AF218073 | 640 | 102 | O78:K80 Av 89- |
| | | | | 7098(143) |
| uidA | S69414 | 250 | 103 | O157:H7 EDL933 |
| uspA | AB027193 | 501 | 104 | h140 8550 |

Note:
Amplicons were prepared using primers noted herein and strains noted above as source of template for PCR amplification

Tables 3 and 4 list the antimicrobial resistance genes and mutations thereof tested, as well as their origin from specific control strain identified by name and accession number.

TABLE 3
Antimicrobial Resistance Genes used

| | Accession | |
|---|---|---|
| Gene | Number | Control Strain |
| bla_{TEM} | AF309824 | R6K |
| bla_{SHV} | AF117743 | pMON38 |
| bla_{OXA-1} | AJ238349 | pMON300 |
| bla_{OXA-7} | X75562 | pMG202 |
| bla_{PSE-4} | J05162 | pMON711 |
| bla_{CTX-M-3} | X92506 | CCRI-2167 |
| ant(3″)-Ia (aadA1) | X12870 | ETEC074 |
| aph(3′)-Ia (aphA1) | AF330699 | Tn903 |
| aph(3′)-IIa (aphA2) | V00618 | Tn5 (M155) |
| aac(3′)-II (aacC2) | X13543 | R176 |
| aac(6″)-I (aacA7) | U13880 | pMAQ135 |
| ant(2″)-Ia (aadB1) | X04555 | PM203 (tn 1409) |
| tet(A) | X00006 | SAS1393 (RP4) |
| tet(B) | L20800 | CT4afooB (Tn 10) |
| tet(C) | J01749 | pBR322 |
| tet(D) | X65876 | D7-5 (RA1) |
| tet(E) | L06940 | pSL1540 |
| tet(Y) | AF070999 | AF070999 |
| catI | M62822 | pBR325 |
| catII | X53796 | RSA |
| catIII | X07848 | pUC18:IM3:Clal |
| floR | AF252855 | CVM1817 |
| dhfrI | X00926 | S17-1 lamda pir |
| dhfrV | X12868 | pLM020 |
| dhfrVII | X58425 | pLM027 |
| dhfrIX | X57730 | C600 |
| dhfrXIII | Z50802 | Dhfr13 |
| dhfrXV | Z83311 | Dhfr15 |
| suII | X12869 | PACYC184 |
| suIII | M36657 | RSF1010 |
| Class 1 integron | X12870 | ETEC074 |

TABLE 4
Mutation of Antimicrobial Resistance Genes

| | | |
|---|---|---|
| Gene | Mutation | Probe sequence |

| | | |
|---|---|---|
| gyrA | D87V | tic gga cga tcg tga cat a |
| | D87H | tic gga cga tcg tga cat a |
| | D87Y | tic gga cga tcg tgt aat a |
| | D87G | tic gga cga tcg tgc cat a |
| | D87N | tic gga cga tcg tgt tat a |
| | A84P | atc gtg tca tai aci ggc ga |
| | S83W | gtg tca tai aci gcc cag tc |
| | S83A | gtg tca tai aci gcc gcg tc |
| | S83L | gtg tca tai aci gcc aag tc |
| | D82G | tca tai aci gcc gag cca cc |
| | G81D | tai aci gcc gag tca tca tg |
| | G81C | tai aci gcc gag tca caa tg |
| gyrB | Lys447Glu | att tta ccc tcc agc ggc |
| parC | Ser80Ile | ata aca ggc gat atc gcc gtg |
| | Ser80Arg | ata aca ggc tct atc gcc gtg |
| | Ser80Leu | ata aca ggc gag atc gcc gtg |
| | Glu84Lys | agg acc atc gct tta taa ca |
| | Glu84Gly | agg acc atc gct cca taa ca |
| | Glu84Val | agg acc atc gct aca taa ca |

Selection and Sequence Analysis of Virulence Gene Probes

The selection included virulence genes of E. coli pathotypes involved in intestinal and extra-intestinal diseases in humans and animals (see Table 2). The primers used for probe amplification were either chosen from previous studies on virulence gene detection or designed from available gene sequences (see Table 5). One hundred three E. coli virulence genes were targeted in this study, encoding (a) toxins (heat-labile toxin LT, human heat-stable toxin STaH, porcine heat-stable toxin STaP, Shiga-toxins Stx1 and Stx2, haemolysins Hly and Ehx, East1, STb, EspA, EspB, EspC, cytolethal distending toxin Cdt, cytotoxic necrosing factor Cnf, Cva, Leo) (b) adhesion factors (Cfa, Iha, Pap, Sfa, Tir, Bfp, Eaf, Eae, Agg, Lng, Aida, Foc, Afa, Nfa, Drb, Fim, Bma, ClpG, F4, F5, F6, F17, F18, F41) (c) secretion systems (Etp) (d) capsule antigens (KfiB, KpsMTII, KpsMTIII, Neu) (e) somatic antigens (RfcO4, RfbO9, RfbO101, RfbO111, RfbEO157) (f) flagellar antigen (FliC), (g) invasins (IbeA, IpaC, InvX), (h) autotransporters (Tsh), (i) aerobactin system (lucD, TraT, lutA) and, in addition, to espP (serine-protease), katP (catalase), omp (outer membrane proteins A and T), iroN (catechol siderophore receptor), iss (serum survival gene), putative RTX family exoprotein (rtx) and paa (related attaching and effacing gene) probes. The Yersinia high-pathogenicity island (ifp1, irp2, and fyuA) present in different E. coli pathotypes and other Enterobacteriaceae was also targeted. An E. coli positive control gene, uidA, which encodes the E. coli-specific 6-glucuronidase protein and the uspA gene which encodes a uropathogenic-specific protein were added to this collection.

TABLE 5
DNA Sequences of primers designed

| | | SEQ | | SEQ |
|---|---|---|---|---|
| | | ID | | ID |
| Gene | Forward | NO: | REVERSE | NO: |
| afaE5 | GCGATCATGGCCGCGACCAGCA | 105 | CAACTCACCCAGTAGCCCCAGT | 106 |
| cdt2 | GAAAGTAAATGGAATATAAATG | 107 | TTTGTGTTGCCGCCGCTGGTGAA | 108 |
| cdt3 | GAAAGTAAATGGAATATAAATG | 109 | TTTGTGTCGGTGCAGCAGGGAAA | 110 |
| cfaI | GGTGCAATGGCTCTGACCACA | 111 | GTCATTACAAGAGATACTACT | 112 |
| cs1 | GCTCACACCATCAACACCGTT | 113 | CGTTGACTTAGTCAGGATAAT | 114 |
| cs3 | GGGCCCACTCTAACCAAAGAA | 115 | CGGTAATTACCTGAAACTAAA | 116 |
| derb122 | CGTGTGGGAGCCCTGAGCCTT | 117 | CCGGCCTGGTTGCTAGTATT | 118 |
| espA group I | CATCAGTTGCTAGTGCGAATG | 119 | CAGCAAATGTCAAATACGTT | 120 |
| espA group II | CGACATCGACGATCTATGACT | 121 | CCAAGGGATATTGCTGAAATA | 122 |
| espA group III | CATCAGTTGCTAGTGCGAATG | 123 | CAGCAAATGTCAAATACGTT | 124 |
| espB group I | CGGAGAGTACGACCGGCGCTT | 125 | GCACGGCTGGCTGCTTTCGTT | 126 |
| espB group II | GCTGCCATTAATAGCGCAACT | 127 | TATTGTTGTTACCAGCCTTGC | 128 |
| espB group III | GTAATGACGGTTAATTCTGTT | 129 | GCCGCATCAATAGCCTTAGAA | 130 |
| espC | CCCATAACGGAACAACTCAT | 131 | CAGAATAGACCAAACATCTGCA | 132 |
| etpD | GGCCACTTTCAATGTTGGTCA | 133 | CGACTGCACCTGTTCCTGATTA | 134 |
| invX | TCTGATATAGTTTATATGGGT | 135 | TCAAACCCCACTCTTAATTAA | 136 |
| ipaC | TTGCAAAAGCAATTTTGCAAC | 137 | TGCCGAACAATGTTCTCTGCA | 138 |
| kfiB | AATTGTTTTAAAATCTGTTCT | 139 | TGAGACTGAAATTACATTTAA | 140 |
| leoA | GAACAATTCAAACAGTTCAGT | 141 | TTATTCAAATCGCGCAATACC | 142 |
| lngA | CAAATACAGTCCGCGTACGA | 143 | CCATTGTTACCTAAAGAGCGT | 144 |
| neuC | TTGGCAGTTACAGGAATGCAT | 145 | AACAGTGAACCATATTTTAGT | 146 |
| paa | ATGAGGAACATAATGGCAGG | 147 | TCTGGTCAGGTCGTCAATAC | 148 |
| rfbO9 | GGTGATCGATTATTCCGCTGA | 149 | ACGCCTCATCGGTCAGCGCCT | 150 |
| ribO101 | TCTGCACGTTTAAAATTATTG | 151 | GTTTCTCCGTCAGAATCAAGC | 152 |
| rtx | CTACCGTAGCGGGCGATGGTA | 153 | CAGCGCCTGTCCGTGTTCGGC | 154 |
| sfaA | CCCTGACCTTGGGTGTTGCGA | 155 | GTACTGAACTTTAAAGGTGG | 156 |
| stah | AAGAAATCAATATTATTTAT | 157 | AATAGCACCCGGTACAAG | 158 |
| stxA I | GCGAAGGAATTTACCTTAGA | 159 | CAGCTGTCACAGTAACAAAC | 160 |
| stxA II | CTTGAACATATATCTCAGGG | 161 | ACAGGAGCAGTTTCAGACAGT | 162 |
| stxB I | GGTGGAGTATACAAAATATAA | 163 | ATGACAGGCATTAGTTTTAAT | 164 |
| stx B II | TTCTGTTAATGCAATGGCGG | 165 | TTCAGCAAATCCGGAGCCTGA | 166 |
| stxB III | GAAGAAGATGTTTATAGCGG | 167 | ACTGCAGGTATTAGATATGAT | 168 |
| tir group I | ATTGGTGCCGGTGTTACTGCTG | 169 | CTCCCATACCTAAACGCAAT | 170 |
| tir group II | ATTGGTGTTGCCGTCACCGCT | 171 | ACGCCATGACATGGGAGG | 172 |
| tir group III | ATTGGTGCTGGTGTAACGACT | 173 | ATTGCGTTTAGGTATGGG | 174 |
| uspA | CTACTGTTCCCGAGTAGTGTG | 175 | GGTGCCGTCCGGAATCGGCGT | 176 |

The selection included antibiotic resistance genes (see Table 6).

TABLE 6
Antimicrobial Resistance Genes

| | | | Antimicrobial |
|---|---|---|---|
| | Gene Family | Resistance Gene | Resistance |
| Gram | Aminoglycosides | ant(3″)-Ia, ant(2″)-Ia, | Kanamycin, |
| Negative | | aac(3)-IIa, aac(3)-IV, | neomycin, |
| | | aph(3′)-Ia, aph(3′)-IIa | gentamicin |
| | Beta-Lactams | bIa_{TEM}, bla_{SHV}, | Ampicilün, |
| | | bla_{OXA-1}, bla_{OXA-7}, | cephalosporins |
| | | bla_{PSE-4}, bla_{CTX-M-3} | class I, II, III |
| | Phenicols | catI, catII, catIII, floR | Chloramphenicol, |
| | | | florfenicol |
| | Tetracyclines | tet(A), tet(B), tet(C), | Tetracycline, |
| | | tet(D), tet(E), tet(Y) | oxytetracycline |
| | Trimethoprims | dhfrI, dhfrV, dhfrVII, | Trimethoprim |
| | | dhfrIX, dhfrXIII, | |
| | | dhfrXV | |
| | Sulfonamides | suII, suIII | Sulfonamide |

TABLE 7
List of oligonucleotides probes used in an embodiment of the micrroarray

| | | Oligo | Length | | | | | resultats | | souche | |
|---|---|---|---|---|---|---|---|---|---|---|---|
| | | probe | of | | | G + C | Accession | BLAST | Nom | de | patho- |
| Gene | Fonction | (5′ to 3′) | sequence | Position | Tm | content | number | (croisements) | l'oligo | référence | types |
| aap | dispersine (proteine anti- | TTG GGA CGG GTC | 70 | 121- | 73.7 | 55.7 | Z32523 | aucun | 70-aap121 | 17.2 | EAEC |
| | aggragative), autre nom: | CAC ATT ATC TGC | | 52 | | | | | | (SB48) | |
| | aspU (EAEC secreted | GTT CCA ACC GCT | | | | | | | | | |
| | prot U) | ACC ACC CGC AAA | | | | | | | | | |
| | | GGC ATT CAG GCT | | | | | | | | | |
| | | GAT ACC CAA G | | | | | | | | | |
| aatA | proteine de transport et | TTC CTC CTC CTC | 70 | 3130- | 64.6 | 30 | AY351860 | aucun | 70-aatA3130 | 17.2 | EAEC |
| | d'export de aap (ABC | AAG TAC ATC AAT | | 3061 | | | | | | (SB48) | |
| | transporter system), | ATC AAA CCT GAT | | | | | | | | | |
| | plasmide pAA2 des EAEC | TTT TTG TAA TAT | | | | | | | | | |
| | (similaire \grave{a}tolC) | ATT ATA TCT CAT | | | | | | | | | |
| | | CTC TAC ATC A | | | | | | | | | |
| aggA | sous-unite fimbriale majeure | ACA ATC ATT TGT | 70 | 4131- | 74.3 | 42.9 | U12894 | aucun | 70-aggA4131 | 17.2 | EAEC |
| | (AAF/I: aggregative | AAC GGT GAG GCG | | 4062 | | | | | | (SB48) | |
| | adherence fimbriae I) | GAT TGT CTC AGT | | | | | | | | | |
| | | TGC TTT TAT TGG | | | | | | | | | |
| | | AGG TCT TTC TAA | | | | | | | | | |
| | | CGC AGC GTT A | | | | | | | | | |
| aafA | sous unite fimbriale majeure | CCA GCA TCA GCG | 70 | 2831- | 77 | 55 | AF012835 | aucun | 70-aafA2831 | 042 | EAEC |
| | (AAF/II) et adhesine | CAG CGT TGC GGT | | 2762 | | | | | | | |
| | | TGT CTA ATA GTA | | | | | | | | | |
| | | AAA CTC AGG TCG | | | | | | | | | |
| | | ATA TTT GCG CTC | | | | | | | | | |
| | | CTG TCA ACG T | | | | | | | | | |
| aag3A | sous unite fimbriale majeure | CTG TAA TAA CTG | 70 | 4340- | 68.5 | 44.3 | AF411067 | aucun | 70-agg | 55989 | EAEC |
| | (AAF/III) | GAT CCC GCT GCT | | 4271 | | | | | 3A4340 | | |
| | | ATA GAT AAC CCA | | | | | | | | | |
| | | CTG TAC AAG CTG | | | | | | | | | |
| | | AAT ACC AGA CTC | | | | | | | | | |
| | | GCA ATG ATA C | | | | | | | | | |
| agn43 | antigene 43, adhesine qul | TGT CGT TCA GCG | 70 | 4205- | 73.7 | 54.3 | U24429 | aucun | 70-agn(43) | ML 308- | commun |
| | confere des capacites | TCA GCG TGC CTT | | 4136 | | | | | 4205 | 225 | |
| | d'aggregation c-c. autre | CAT TCA GGT TGA | | | | | | | | | |
| | nom: flu (fluffing prot) | CGG CTT TCT GGG | | | | | | | | | |
| | | TGA GTG TGG TGT | | | | | | | | | |
| | | TGC TGA CAG T | | | | | | | | | |
| afaD | sous unité mineure des AFA, | CCT GAC CGG GCC | 70 | 7788- | 77.3 | 68.6 | X76888 | AFAD (1,2,3,5), | 70-afaD7788 | A22 | commun |
| | invastine | TCG ACA CCC CCT | | 7719 | | | | dafaD, draD, daaD | | (SB53) | |
| | | TCC CGC CTT CTC | | | | | | | | | |
| | | CCT TCA CCG GCG | | | | | | | | | |
| | | ACC AGC CAT CTC | | | | | | | | | |
| | | CTC CTG TCC T | | | | | | | | | |
| afaE1 | sous unité majeure des | CCC GTT GGT GCC | 70 | 250- | 75.2 | 58.6 | X69197 | dafaE (AFA de | 70-afaE(1) | KS52 | commun |
| | AFA-I | GCT GCT GGT AAA | | 181 | | | | EPEC) | 250 | | |
| | | ATT GGC TTG AGC | | | | | | | | | |
| | | GGT GCC GGT CAT | | | | | | | | | |
| | | CAT CAT TAC GCT | | | | | | | | | |
| | | GGT TGC GCC T | | | | | | | | | |
| afaE2 | sous unite majeure des | GCC TGT TGC GTG | 70 | 250- | 72.6 | 52.9 | X85782 | aucun | 70-afaE(2) | A22 | commun |
| | AFA-II | TTT ATC CAC CGC | | 281 | | | | | 250 | (SB53) | |
| | | TGC GTG CGT AGT | | | | | | | | | |
| | | CCC AAC AAA GGT | | | | | | | | | |
| | | CCC GCA TAG TAT | | | | | | | | | |
| | | CAT GGT CAT A | | | | | | | | | |
| afaE3 | sous unité majeure des | TGG TGC CAC TCG | 70 | 8730- | 78.4 | 67.1 | X76888 | draE (Dr) | 70-afaE(3) | A22 | commun |
| | AFA-III | GGG TGA ACC CAG | | 8661 | | | | | 8730 | (SB53) | |
| | | CAT GCG CGG AGC | | | | | | | | | |
| | | TCA CGG CGA ACA | | | | | | | | | |
| | | CCA TGC TGG CCG | | | | | | | | | |
| | | CGG CCA TGA T | | | | | | | | | |
| afaE5 | sous-unite majeure (AFA-V) | GTA TTC CAC GCA | 70 | 507- | 80.9 | 58.6 | X91748 | aucun | 70-afaE(5) | AL851 | commun |
| | | CGC CCG TCG GTG | | 438 | | | | | 507 | (SB52) | |
| | | GCC TGC AAG CGG | | | | | | | | | |
| | | ACA TTT ATC CGT | | | | | | | | | |
| | | GCC TGA TAG TCA | | | | | | | | | |
| | | TCG CGG ATC A | | | | | | | | | |
| afaE7 | sous-unite majeure | ACA TCA ACA GTT | 70 | 4118- | 74.1 | 41.4 | AF072901 | aucun | 70-afaE(7) | 262- | commun |
| | (AFA-VII) | GAT TTA GCT GCA | | 4049 | | | | | 4118 | KH89 | |
| | | AGA GCA TTA AAG | | | | | | | | (SB41) | |
| | | GAC AGC GCA ATA | | | | | | | | | |
| | | AGT CCG ATG GTT | | | | | | | | | |
| | | AAA GCA TGC T | | | | | | | | | |
| afaD8 | invasine, AFA-8 | CAA CTG CCT GCG | 70 | 4892- | 72.2 | 41.4 | AF072900 | aucun | 70-afaD(8) | 2787 | commun |
| | | CCA GAC TGG ATA | | 4823 | | | | | 4892 | (SB16) | |
| | | TAA CCA CCA GTA | | | | | | | | | |
| | | CAA TAC CAC TAC | | | | | | | | | |
| | | ATA CTA TCT GTA | | | | | | | | | |
| | | TTT TCT TCA T | | | | | | | | | |
| daaE | sous-unite majeure des | GGC ACT CTT CGG | 70 | 430- | 74.8 | 60 | M27725 | aucun | 70-daaE430 | C1845 | commun |
| | F1845 (famille Dr) | TCA CAG TCA GTG | | 361 | | | | | | | |
| | | TGG TAA TAC CCG | | | | | | | | | |
| | | TTG TCC CGC TCG | | | | | | | | | |
| | | CTT GGA ACG TGG | | | | | | | | | |
| | | CTT GCG CGG A | | | | | | | | | |
| drbE(121) | sous-unite majeure | TTT GCT ATG AGC | 70 | 149- | 78.9 | 54.3 | U87540 | aucun | 70-drbE | F56-62 | commun |
| | (adhesine de la famille Dr), | TTT CCT ACA GTT | | 80 | | | | | (121)149 | | |
| | soustype 121 | ACT GGG CAT TCG | | | | | | | | | |
| | | CCA GTC ACC GTT | | | | | | | | | |
| | | AGT TCC ACG CCC | | | | | | | | | |
| | | CCT GTG GTC C | | | | | | | | | |
| drbE(122) | sous-unite majeure (Dr), | ATT GGC CCC CAT | 70 | 340- | 80 | 54.3 | U87541 | aucun | 70-drbE | J96 | commun |
| | soustype122 | CGG ATG CCA CCA | | 271 | | | | | (122)340 | O4:K12 | |
| | | AGC GCA CAT TTA | | | | | | | | (SB18) | |
| | | TCC GCG CTT GTT | | | | | | | | | |
| | | GGT CTT CAC GTA | | | | | | | | | |
| | | GCA GTA CGA T | | | | | | | | | |
| nfaA | sous-unité majeure des | TTA AGG TAA AAC | 70 | 506- | 78 | 51.4 | S61970 | dra2E (DrII), | 70-nfaA506 | 31A | UPEC |
| | NFAI | TTG TTG GTC ACC | | 437 | | | | nfaE116 (adhésine | | (SB13) | |
| | | GTA GTG CCC TGC | | | | | | NFAE116 de la | | | |
| | | GCG ACC CCC TGT | | | | | | famille Dr) | | | |
| | | CCT TCG CCA TCG | | | | | | | | | |
| | | ATC TCT TTA A | | | | | | | | | |
| nfaE111 | sous-unité majeure des | AGC GTC AGG GGT | 70 | 210- | 79.6 | 54.3 | U87790 | aucun | 70-nfaE | 1069-11 | UPEC |
| | adhésines NFAE-111 | AGC GAT TGT CAG | | 141 | | | | | (111)210 | | |
| | (famille Dr) | ATT TAC TGT GCA | | | | | | | | | |
| | | GCT TTC CAT GTT | | | | | | | | | |
| | | GGT GAT CGT CCC | | | | | | | | | |
| | | GCT CGC GGT T | | | | | | | | | |
| aida1 | adhesine (adherence diffuse | GAT TGT GGA AAC | 70 | 177- | 74.6 | 44.3 | X65022 | aucun | 70-aida(1) | 2787 | DAEC |
| | chez EPEC) | AAC CGC CAA TAC | | 108 | | | | | 177 | (SB16) | |
| | | CAG CAG TGT ATT | | | | | | | | | |
| | | TTT TGC AAG GAC | | | | | | | | | |
| | | AAA ACC ATG TCC | | | | | | | | | |
| | | TCT GGC TAA C | | | | | | | | | |
| afrA | ssu majeure des pili AF/R1 | AAG ACC ATG CCA | 70 | 2245- | 71.4 | 47.1 | AF050217 | aucun | 70-afrA2245 | RDEC | rabbit |
| | (REPEC) | TTT TAG CAG TAG | | 2176 | | | | | | | entéro- |
| | | TGA TGG TAT TGC | | | | | | | | | adhérente |
| | | ATG TCA CCC CTG | | | | | | | | | |
| | | ATG CTG GCT TCA | | | | | | | | | |
| | | GGG TAA ACG A | | | | | | | | | |
| afr2G | ssu majeure des pili AF/R2 | TGT CAG AGA ACC | 70 | 550- | 70.8 | 45.7 | U77302 | aucun | 70-afr2G550 | B10 | REPEC |
| | (REPEC) | GAT AGT AGC CTT | | 481 | | | | | | | |
| | | TGA TTC ATC TTT | | | | | | | | | |
| | | AAT TGG CAA CGT | | | | | | | | | |
| | | CAG ACT TGC CTT | | | | | | | | | |
| | | GCC CTG GCT T | | | | | | | | | |
| artJ | arginine-binding | AGC TTT AAT TGC | 70 | 4030- | 71.7 | 48.6 | X86160 | aucun | 70-artJ4030 | EDL933 | commun |
| | periplasmic protein, | TGC CAG CGC GTT | | 3961 | | | | | | O157:H7 | |
| | supposée impliquée dans | ATT CAG TTT TTC | | | | | | | | (SB44) | |
| | urovirulence | CAG CAG GGC TTT | | | | | | | | | |
| | | GTT ATG CGG ACG | | | | | | | | | |
| | | TAC AGC GAT G | | | | | | | | | |
| bfpA | sous-unite fimbriale majeure | CAA GCA CCA TTG | 70 | 2783- | 69.5 | 32.9 | U27184 | tous les variants | 70-bfpA2783 | E2348/69 | EPEC |
| | (BFP: bundle-forming pili) | CAG ATT CAA TCA | | 2714 | | | | (a1,a2,a3,β1,β2, | | O126:H6 | |
| | | AAG ACA GAC CTT | | | | | | β3,β4,β5,β6) | | (SB28) | |
| | | TTT CGT ATT TCT | | | | | | | | | |
| | | TAT TCA TGA TTT | | | | | | | | | |
| | | TAG AAA CCA T | | | | | | | | | |
| bfpA | sous unité majeure des BFP | AGC AGT CGA TTT | 70 | 539- | 68.2 | 38.6 | AF304481 | tous les variants | 70-bfpA | E2348/69 | EPEC |
| alpha | (variants alpha) | AGC AGC CTG ATC | | 470 | | | | alpha | alpha539 | O126:H6 | |
| | | AGC GCT ATT ACC | | | | | | | | (SB28) | |
| | | AAA TGA TGT AAT | | | | | | | | | |
| | | GTT ATT TTC GCC | | | | | | | | | |
| | | AGA GAT ATT A | | | | | | | | | |
| bfA | sous unité majeure des BFP | GCC TCA GCA GGA | 70 | 546- | 67.9 | 42.9 | AF474407 | tous les variants | 70-bfpA | RN587/1 | EPEC |
| beta | (variants beta) | GTA ATA GCT GAC | | 477 | | | | beta | beta546 | | |
| | | GAT TTA GCG TTA | | | | | | | | | |
| | | CCA CTA GTG GCT | | | | | | | | | |
| | | GAA GTA TTA AAT | | | | | | | | | |
| | | GAA GTA GTA G | | | | | | | | | |
| bmaE | sous-unité majeure de la M- | CAT GGC AAG TTA | 70 | 97- | 72.2 | 38.6 | M15677 | afaE8 (AFA-VIII) | 70-bmaE97 | B83-215 | UPEC |
| | agglutinine (BmaE) | GCG CCA TTG TTA | | 28 | | | | genes 100% | | (SB25) | |
| | | TAC CTG CAA AGA | | | | | | identiques | | | |
| | | CAC TGC TTG CGA | | | | | | | | | |
| | | TAG CTA TTT TCT | | | | | | | | | |
| | | TTA AAT TCA T | | | | | | | | | |
| capU | cap locus protein, | ATG AAC TAT TCC | 70 | 1630- | 67.3 | 37.1 | AF134403 | aucun | 70-capU1630 | 042 | EAEC, |
| | hexosyltransferase (related | GAG TAA TCT CCA | | 1561 | | | | | | | DAEC |
| | LPS biosynthesis gene), | TAC AGT AGG AAT | | | | | | | | | |
| | plasmide pAA2 des EAEC | GTG AAG ACT GTT | | | | | | | | | |
| | | TCG AAA TAA CGC | | | | | | | | | |
| | | GAA TGT GAT A | | | | | | | | | |
| caa | gène structural de la | TAA AAC CCG TGT | 70 | 3589- | 71.0 | 48.6 | M37402 | aucun | 70-caa3589 | | |
| | colicine A | AAA CCC TCT GCC | | 3520 | | | | | | | |
| | | GTA AGG AAC CAT | | | | | | | | | |
| | | CGA TGA ATT ATC | | | | | | | | | |
| | | AGC GGT CAT CAC | | | | | | | | | |
| | | CGT TCC GTT C | | | | | | | | | |
| cba | gene structural de la | AAA ACC AAC AAC | 70 | 1970- | 70.3 | 42.9 | M16816 | aucun | 70-cba1970 | | |
| | colicine B | TGT GGC CGA AAG | | 1901 | | | | | | | |
| | | ACC AAA GGC TAT | | | | | | | | | |
| | | AAG GGC CGA GCC | | | | | | | | | |
| | | TAA TGT CAA AGA | | | | | | | | | |
| | | AAA CAA ACT A | | | | | | | | | |
| cda | gene structural de la | AAA CAG GAG TAA | 70 | 1714- | 70 | 45.7 | Y10412 | aucun | 70-cda1714 | | |
| | colicine D | TCG TCG TTA CTG | | 1645 | | | | | | | |
| | | GCA TTT CGA CCG | | | | | | | | | |
| | | GTT TTA CTT CCG | | | | | | | | | |
| | | TTC CTG TAT GCA | | | | | | | | | |
| | | CTG GTG TAA C | | | | | | | | | |
| cela | gene structural de la | CAC TCC CGT CAG | 70 | 297- | 67.6 | 41.4 | J01563 | aucun | 70-ce1a297 | CFT073 | |
| | colicine E1 | GAG TAC CAT TCA | | 228 | | | | | | | |
| | | AAA GAG TAA TAA | | | | | | | | | |
| | | TTA CCT GCT CCT | | | | | | | | | |
| | | TAT CAT CAT AAG | | | | | | | | | |
| | | GAA CAC CAT C | | | | | | | | | |
| ceil | gene structural de la | TCT TTT GCA GCA | 70 | 1253- | 71.4 | 47.1 | X12591 | ceab (E2), ceac (E3), | 70-cei1253 | | |
| | colicine E9, autre nom: | GCA TCA AAT GCA | | 1184 | | | | colE4 (E4), colE5 | | | |
| | colE9, ce9a | GCC TTC TTA TTA | | | | | | (E5), colE6 (E6), | | | |
| | | TTT ACA TCC GTC | | | | | | colE7 (E7), colE8 (E8) | | | |
| | | TGC GCC CGC TGA | | | | | | | | | |
| | | GCT TTA AGC C | | | | | | | | | |
| cia | gene structural de la | TGT CAG CCC GGT | 70 | 2439- | 68.5 | 40 | M13819 | aucun | 70-cia2439 | | |
| | colicine 1a | ACT TTT CAT ACG | | 2370 | | | | | | | |
| | | TTT TTA ATG CCT | | | | | | | | | |
| | | CTT CAA CAT TAC | | | | | | | | | |
| | | GTA TTT TCT TCC | | | | | | | | | |
| | | CTT TAG CCT G | | | | | | | | | |
| cib | gene structural de la | GAT TAT TAC GGA | 70 | 2440- | 66.9 | 37.1 | X01009 | aucun | 70-cib2440 | | |
| | colicine 1b | ATT TAT CAA AAG | | 2371 | | | | | | | |
| | | CGT TCA GTG CAT | | | | | | | | | |
| | | CAT CCA CAC TCT | | | | | | | | | |
| | | TAA TCT GTT TCC | | | | | | | | | |
| | | CTT GAG ATA C | | | | | | | | | |
| cka | gene structural de la | CAC TAA TCT GTG | 70 | 569- | 65.8 | 31.4 | X87834 | aucun | 70-cka569 | | |
| | colicine K | TAG CAA TTT TAT | | 500 | | | | | | | |
| | | TCT TCT GCT TTT | | | | | | | | | |
| | | GTT TTT CAT TAA | | | | | | | | | |
| | | TTA CAT TAC TCA | | | | | | | | | |
| | | CCA CCT TCG A | | | | | | | | | |
| cma | gene structural de la | TGC ACC ATT GCC | 70 | 125- | 69.2 | 40 | M16754 | aucun | 70-cma125 | | |
| | colicine M | ATA ACT TGG TAA | | 56 | | | | | | | |
| | | GTT AGT TGA TGG | | | | | | | | | |
| | | TGA TGG TGC ATG | | | | | | | | | |
| | | AAC AGT TAA GGT | | | | | | | | | |
| | | TTC CAT ACA T | | | | | | | | | |
| cna | gene structural de la | TAC CAA TGC CCG | 70 | 685- | 70.9 | 44.3 | Y00533 | aucun | 70-cna685 | | |
| | colicine N | GAT TTT TCC CTC | | 616 | | | | | | | |
| | | CAC CAA AAG CAT | | | | | | | | | |
| | | TGT TAT GTG CAT | | | | | | | | | |
| | | TAT CTG CGC CAT | | | | | | | | | |
| | | TAC TAC CCA T | | | | | | | | | |
| csa | gene structural de la | TTG ATT TTT TCC | 70 | 945- | 69.6 | 42.9 | Y18684 | aucun | 70-csa945 | | |
| | colicine S4 | ATA ATA CCC GCC | | 876 | | | | | | | |
| | | TTA GCT TTT TCA | | | | | | | | | |
| | | CTC CCT ACG TAA | | | | | | | | | |
| | | GGA CGG ACA CCT | | | | | | | | | |
| | | GTT CGA AGA A | | | | | | | | | |
| colY | gene structural de la | ATA ATA ATA CCG | 70 | 2830- | 71.9 | 51.4 | AF197335 | aucun | 70-colY2830 | | |
| | colicine Y | ATA ATC CCT ACG | | 2761 | | | | | | | |
| | | ACT GCA GCT GAT | | | | | | | | | |
| | | GCC CCC ACA GCA | | | | | | | | | |
| | | AGC AGG TAA GCT | | | | | | | | | |
| | | CCA AGG GTG G | | | | | | | | | |
| col5 | gene structural de la | CAC CAA ATG CTC | 70 | 382- | 72.5 | 52.9 | X87835 | cta (colicine 10) | 70-col(5) | | |
| | colicine 5 | CAC CGC CTC CAC | | 313 | | | | | 382 | | |
| | | CAA CAT TTT CAG | | | | | | | | | |
| | | TTC CAG TTG CAA | | | | | | | | | |
| | | GCG TCG CTG TAA | | | | | | | | | |
| | | TCG TAT CGC C | | | | | | | | | |
| ccdB | proteine cytatoxique, autres | TCA GCC ACT TCT | 70 | 320- | 74.5 | 57.1 | L27082 | aucun | 70-ccdB320 | EDL933 | EHEC |
| | noms: letB ou proteine G | TCC CCG ATA ACG | | 251 | | | | | | O157:H7 | |
| | | GAG ACC GGC ACA | | | | | | | | (SB44) | |
| | | CTG GCC ATA TCG | | | | | | | | | |
| | | GTG GTC ATC ATG | | | | | | | | | |
| | | CGC CAG CTT T | | | | | | | | | |
| cdtB-1 | sous-unité b (cytolethal | GGT TGC AAC TTT | 70 | 988- | 69.2 | 31.4 | U03293 | aucun | 70-cdtB(1) | S5 | cell- |
| | distending toxin I) | AAA ATC GCT TAA | | 919 | | | | | 988 | O15:KRV | detaching |
| | | ATC TGC AAA AGA | | | | | | | | C383 | EC |
| | | AAT ACC CGG CAA | | | | | | | | (SB12) | |
| | | AAT CAT TAA CAG | | | | | | | | | |
| | | GAA TAA TAA T | | | | | | | | | |
| cdtB-2 | sous-unité b (cytolethal | ATC CAG TTA AGC | 70 | 1743- | 76.4 | 50 | U04208 | cdtB-3 | 70-cdtB(2) | S5 | cell |
| | distending toxin II) | GCC TGG TGT ACT | | 1674 | | | | | 1743 | O15:KRV | detaching |
| | | GGG TCT CTG CTG | | | | | | | | C383 | EC |
| | | TCG CGG AAG AAG | | | | | | | | (SB12) | |
| | | TTA TAC ACT TCC | | | | | | | | | |
| | | TCA ACA AGA G | | | | | | | | | |
| cdtB-3 | sous-unité b (cytolethal | ATC CAG TTA ATG | 70 | 2417- | 75.7 | 48.6 | U89305 | cdtB-2 | 70-cdtB(3) | S5 | commun |
| | distending toxin III), autre | GCC TGG TGG ACT | | 2348 | | | | | 2417 | O15:KRV | |
| | nom: cdt-IIIB | GGG TCT CGG CTG | | | | | | | | C383 | |
| | | TCA CGA AAG AAG | | | | | | | | (SB12) | |
| | | CTA TAG ACT TCT | | | | | | | | | |
| | | TCA ACA AGA G | | | | | | | | | |
| cdtB- | sous-unité b (cytolethal | ATC CAG TTA AGC | 70 | 491- | 70.3 | 45.7 | AY423896 | cdtB-2 et cdtB-3 | 70-cdtB | S5 | commun |
| 2/3 | distending toxin II/III), | GCT TGG TGT ACT | | 422 | | | | | (2/3)491 | O15:KRV | |
| | variant proche des toxines II | GGG TCT CTG CTG | | | | | | | | C383 | |
| | et III | TCG CGG AAG AAG | | | | | | | | (SB12) | |
| | | CTA TAT ACT TCT | | | | | | | | | |
| | | TCA ACA AGT T | | | | | | | | | |
| cdtB-4 | sous-unité b (cytolethal | AGC ATC AGT TCG | 70 | 190- | 68.4 | 40 | AY162217 | aucun | 70-cdtB(4) | 28C | commun |
| | distending toxin IV) | CGA AAA ATA AAT | | 121 | | | | | 190 | | |
| | | AAA AAG CTG CTG | | | | | | | | | |
| | | TGG ACG GCT ATT | | | | | | | | | |
| | | CGT TCC AGT ATT | | | | | | | | | |
| | | CCA GAT ATA C | | | | | | | | | |
| csgE | chaperonne des curli | TGA TAA ATG GGA | 70 | 1680- | 69.6 | 44.3 | X90754 | aucun | 70-csgE1680 | EDL933 | commun |
| | | AAG TGA CAT TAC | | 1611 | | | | | | O157:H7 | |
| | | GGG TAA CTT AAC | | | | | | | | (SB44) | |
| | | GAT TAA TGA AAG | | | | | | | | | |
| | | GCC CAG TGC ACG | | | | | | | | | |
| | | ATG GGG AAG C | | | | | | | | | |
| cfaB | sous-unite majeure des | TGA TGC GGG AGA | 70 | 261- | 70.4 | 42.9 | S73191 | aucun | 70-cfaB261 | H-10407 | ETEC |
| | CFA/I (autre nom: F2) | ATA AGC TAA CTT | | 192 | | | | | | (SB29) | |
| | | TAC AGC TGA TGG | | | | | | | | | |
| | | CAG AGC ATT GCC | | | | | | | | | |
| | | ATC AGC TTG CAA | | | | | | | | | |
| | | AAG ATC AAA T | | | | | | | | | |
| cooA | sous-unite majeure des CS1 | CTA ATG GTC TTC | 70 | 473- | 71 | 45.7 | M58550 | aucun | 70-cooA473 | PB-176P | ETEC |
| | (CFA/I like), autre nom: | TCG ACC GCA GAT | | 404 | | | | | | (SB30) | |
| | csoA | GCT CCC ATA GTT | | | | | | | | | |
| | | GCA AAT AAT GTC | | | | | | | | | |
| | | GCC AGA GCC ATT | | | | | | | | | |
| | | GCG CCA ATT G | | | | | | | | | |
| cotA | sous-unite majeure des CS2 | CAG CAG AAG CCC | 70 | 1324- | 71 | 37.1 | Z47800 | aucun | 70-cotA1324 | C91f-6 | ETEC |
| | (CFA/I like) | CCA TGC TAA CAA | | 1255 | | | | | | | |
| | | ATG TAG ATG AAA | | | | | | | | | |
| | | GAA CTA ATG CTC | | | | | | | | | |
| | | CAA TAA TCT TAT | | | | | | | | | |
| | | TGA GTT TCA T | | | | | | | | | |
| cs3 | sous-unite majeure des | CTG CAG CTA GTG | 70 | 151- | 67.6 | 31.4 | M35657 | aucun | 70-cs(3)151 | PB-176 | ETEC |
| | CFA/II (autre noms: CS3 | AGT ATG AAC TCA | | 82 | | | | | | (SB31) | |
| | et F3) | TAG CTG ACA GTG | | | | | | | | | |
| | | AAA GAC CTA TTA | | | | | | | | | |
| | | ATA AGT ATT TTA | | | | | | | | | |
| | | TTT TTA ACA T | | | | | | | | | |
| csfA | sous-unite majeure des CS4 | TTC AAA ACG ACT | 70 | 93- | 73.3 | 42.9 | X97493 | aucun | 70-csfA93 | 9b-1373 | ETEC |
| | (CFA/I like), autre nom: | TGC CGC AGG TGA | | 24 | | | | | | | |
| | csaB | ATA GGT TAA TTC | | | | | | | | | |
| | | TAC AGC AGT AGG | | | | | | | | | |
| | | TAA ACT ACT ACC | | | | | | | | | |
| | | ATC AGC TTG C | | | | | | | | | |
| cs5 | sous-unite majeure des | GAA AAG CGT TCA | 70 | 390- | 71 | 50 | X63411 | aucun | 70-cs(5)390 | PE-423 | ETEC |
| | CFA/IV (autre nom: CS5) | CAC TGT TTA TAT | | 321 | | | | | | | |
| | | TAG CTG ACG TGT | | | | | | | | | |
| | | CAC GCG TAA CCG | | | | | | | | | |
| | | GCG CTC CAG GAG | | | | | | | | | |
| | | TTA CGT TTC C | | | | | | | | | |
| cssA | sous-unite majeure des CS6 | AAT CAT CAG CGG | 70 | 931- | 72.7 | 42.9 | U04844 | aucun | 70-cssA931 | E10703 | ETEC |
| | | TAT TTA CGA GTC | | 862 | | | | | | | |
| | | GTC CTA ACC CAT | | | | | | | | | |
| | | AAT CTT CAT CAT | | | | | | | | | |
| | | AAA CAG GGT AGA | | | | | | | | | |
| | | CCG TTA CCT G | | | | | | | | | |
| csvA | sous-unite majeure des CS7 | GAA AAG CTT TCA | 70 | 483- | 68.7 | 44.3 | AY009095 | aucun | 70-csvA483 | E29101A | ETEC |
| | | CAC TAT TCA TAG | | 414 | | | | | | | |
| | | ATG TCG TAT CAC | | | | | | | | | |
| | | TAC GTG TAA CCG | | | | | | | | | |
| | | GCG ATC CAG CAG | | | | | | | | | |
| | | TTA CTG TTC C | | | | | | | | | |
| cofA | sous-unite majeure des | AGA ATC ACC ACA | 70 | 446- | 76.2 | 48.6 | D37957 | aucun | 70-cofA446 | 260-1 | ETEC |
| | CFA/III (autre nom: CS8) | CCC GCA GCA ATT | | 377 | | | | | | | |
| | | GTT CCG ATA ATC | | | | | | | | | |
| | | CCC AGA ACG ATG | | | | | | | | | |
| | | ATG ACT TCC AGA | | | | | | | | | |
| | | AGG CTC ATA C | | | | | | | | | |
| cswA | sous-unite majeure des CS12 | CCT GGC TTG CAT | 70 | 3610- | 71.2 | 50 | AY009096 | aucun | 70-cswA3610 | 350C1 | ETEC |
| | | CAT TGT TAT TCG | | 3541 | | | | | | | |
| | | CTT GGC CGT TAC | | | | | | | | | |
| | | TAC CGA TCG CAG | | | | | | | | | |
| | | CGA AGG CTG AGC | | | | | | | | | |
| | | TAT TCA TTA G | | | | | | | | | |
| csuA | sous-unite majeure des CS14 | TTT CGG TGT ATC | 70 | 270- | 68.4 | 38.6 | X97491 | csuA1 et csuA2 | 70-csuA270 | E7476A | ETEC |
| | | AAC CAG TCG AAC | | 201 | | | | | | | |
| | | ATC TAA ACC TTT | | | | | | | | | |
| | | TGA AGG GTC ATT | | | | | | | | | |
| | | TGT GTA AAT CTG | | | | | | | | | |
| | | GGT TAG AAC A | | | | | | | | | |
| cs15 | sous-unite majeure des CS15 | GAT ATT ATT CGC | 70 | 585- | 69.2 | 40 | X64623 | aucun | 70-CS(15) | 8786 | ETEC |
| | (antigène 8786), autre | ATT TTG GAA GGC | | 516 | | | | | 585 | | |
| | nom: nfaA | GCG AAT GTC AAG | | | | | | | | | |
| | | ATT AAA ATT ATC | | | | | | | | | |
| | | CTG AGT GCC TGG | | | | | | | | | |
| | | CAA ATG CCA A | | | | | | | | | |
| csbA | sous-unite majeure des CS17 | TGG TAA TTG CCT | 70 | 371- | 72 | 48.6 | X97495 | aucun | 70-csbA371 | E20738A | ETEC |
| | | GCC TCA GGC GCA | | 302 | | | | | | | |
| | | GTT CCT TGT GTG | | | | | | | | | |
| | | TCT GCA TGA ATC | | | | | | | | | |
| | | GTA AGC TGT TGA | | | | | | | | | |
| | | GTG GAA GAA A | | | | | | | | | |
| fotA | sous-unite majeure des CS18 | AGT TAA CCA AGT | 70 | 492- | 68.4 | 40 | U31413 | aucun | 70-fotA492 | ARG-2 | ETEC |
| | (PCFO20) | TAA TTT CGA AGC | | 423 | | | | | | | |
| | | TCT GAG GTT CTC | | | | | | | | | |
| | | CTT TCC CAT TAG | | | | | | | | | |
| | | TTG TAA GAG CTG | | | | | | | | | |
| | | CCT TTG AAA C | | | | | | | | | |
| csdA | sous-unite majeure des CS19 | ACA CCT TGG TAA | 70 | 371- | 71.8 | 48.6 | X97494 | aucun | 70-csdA371 | F595C | ETEC |
| | | TTA CCT GCC TCA | | 302 | | | | | | | |
| | | GGC GGA GCA GCT | | | | | | | | | |
| | | TCT GCA TGA ATC | | | | | | | | | |
| | | GTA AGC TGT TGA | | | | | | | | | |
| | | ACG GAA GAA A | | | | | | | | | |
| csnA | sous-unite majeure des CS20 | AGT TAA TCA GGT | 70 | 250- | 69.8 | 45.7 | AF438157 | aucun | 70-csnA250 | H49A | ETEC |
| | | TAA CCT GAA AGC | | 181 | | | | | | | |
| | | TCT GTG CAG GAC | | | | | | | | | |
| | | TCT TAC CAG TAG | | | | | | | | | |
| | | CTT CCA GTG CGG | | | | | | | | | |
| | | ATT TGG ATA C | | | | | | | | | |
| cseA | sous-unite majeure des CS22 | GAT ATT ATC ATT | 70 | 515- | 65.8 | 31.4 | AF145205 | aucun | 70-cseA515 | ARG-3 | ETEC |
| | | TTT TTG GAA GGC | | 446 | | | | | | | |
| | | TTT AAT ATC AAG | | | | | | | | | |
| | | ACT AAT ATT ATT | | | | | | | | | |
| | | CCC AGC GTC TGG | | | | | | | | | |
| | | CAA ATT CCA A | | | | | | | | | |
| clpG | sous-unite majeure de | TCC CAT TTG TCT | 70 | 222- | 70.7 | 35.7 | M55389 | aucun | 70-clpG222 | 31A | commun |
| | l'antigene de surface CS31A | TTA TAC GCA TCA | | 153 | | | | | | (SB13) | |
| | | GCA GTA ATT GTG | | | | | | | | | |
| | | CCA TTC ATA TCA | | | | | | | | | |
| | | AAT GAA CCA TTA | | | | | | | | | |
| | | AAA TCA CCA G | | | | | | | | | |
| chuA | heme utilization/transport | TTG GCA AGG TGG | 70 | 560- | 76.9 | 47.1 | U67920 | aucun | 70-chuA560 | EDL933 | EHEC, |
| | protein, autre noms: z4911, | CAG AAA CAG CTA | | 491 | | | | | | O157:H7 | UPEC |
| | ecs4380 | AGG CCA ATA AAC | | | | | | | | (SB44) | |
| | | TCA AAC GCA ACG | | | | | | | | | |
| | | AGG TAA ATT GCG | | | | | | | | | |
| | | GAC GTG ACA T | | | | | | | | | |
| cfn1 | cytotoxic necrotizing | TTG AGA AAA GCA | 70 | 1026- | 71.1 | 35.7 | X70670 | aucun | 70-cnf(1) | J96 | UPEC, |
| | factor 1 | GAT GAA ATA AGC | | 957 | | | | | 1026 | O4:K12 | cell- |
| | | ATT ATC AGG ATC | | | | | | | | (SB18) | detaching |
| | | AAT CCG ACT AAA | | | | | | | | | EC |
| | | CCA CGG CAA GTC | | | | | | | | | |
| | | AGT TTT AAA A | | | | | | | | | |
| cfn2 | cytotoxic necrotizing | TTG AGA AAA TCG | 70 | 282- | 68.5 | 31.4 | U01097 | aucun | 70-cnf(2) | S5 | cell- |
| | factor type 2 | TAT AAA ATA AGT | | 213 | | | | | 282 | O15:KRV | detaching |
| | | GTT ATC AGG ATC | | | | | | | | C383 | EC |
| | | CAC TTG ACT AAA | | | | | | | | (SB12) | |
| | | CCA AGG TAA GTC | | | | | | | | | |
| | | TGT TTT GAA A | | | | | | | | | |
| cvaC | gene structural de la | TGT TCC TAT AGC | 70 | 514- | 73.3 | 42.9 | X57525 | aucun | 70-cvaC514 | P84-1195 | commun |
| | microcine V (classe II) | CAT CGC AAT ATC | | 445 | | | | | | O9:K28 | |
| | | ACG CCC TGA AGC | | | | | | | | (SB26) | |
| | | ACC ACC AGA AAC | | | | | | | | | |
| | | AGA ATC TAA TTC | | | | | | | | | |
| | | ATT TAG AGT C | | | | | | | | | |
| mclC | gene structural de la | AAC CCA ATT GAC | 70 | 750- | 66.5 | 32.9 | AY237108 | aucun | 70-mclC750 | | |
| | microcine L (classe II) | ATC ACC AGC ACC | | 681 | | | | | | | |
| | | AGA GAC ATT ATT | | | | | | | | | |
| | | CAT TTC ATT TAA | | | | | | | | | |
| | | CGT TAT TTC TCT | | | | | | | | | |
| | | CAT ATA TCA T | | | | | | | | | |
| mtfS | gene structural de la | GCA AGC GGA TCT | 70 | 1190- | 69.5 | 45.7 | U47048 | aucun | 70-mtfS1190 | | |
| | microcine 24 (classe II) | CCA GCC CCA CCA | | 1121 | | | | | | | |
| | | ACG CAA TTT AAT | | | | | | | | | |
| | | TCC TCT CTA TCT | | | | | | | | | |
| | | AAC TCT CTC ATA | | | | | | | | | |
| | | TAC ATC TCC T | | | | | | | | | |
| mceA | gene structural de la | ATG GAG CTA AGA | 70 | 566- | 68.2 | 40 | AF063590 | aucun | 70-mceA566 | | |
| | microcine E492 (classe II) | ATG AGA GAA ATT | | 497 | | | | | | | |
| | | AGT CAA AAG GAC | | | | | | | | | |
| | | TTA AAT CTT GCT | | | | | | | | | |
| | | TTT GGT GCA GGA | | | | | | | | | |
| | | GAG ACC GAT C | | | | | | | | | |
| mchB | gene structural de la | TAG CTG AAG TCG | 70 | 5578- | 71.4 | 48.6 | AJ009631 | aucun | 70-mchB5578 | CFT073 | |
| | microcine H47 (classe II) | CTG GCG CAC CTC | | 5509 | | | | | | | |
| | | CCG CCC CGG AAA | | | | | | | | | |
| | | TAT ATC TTA ACT | | | | | | | | | |
| | | GTG ATT CTG TTA | | | | | | | | | |
| | | TTT CTC GCA T | | | | | | | | | |
| mcbA | gene structural de la | GAG ACT GGC GTG | 70 | 477- | 68.2 | 38.6 | M24253 | aucun | 70-mcbA477 | | |
| | microcine B17 (classe I) | ATA ATT TAA GAG | | 408 | | | | | | | |
| | | CAT CAA CGG ACA | | | | | | | | | |
| | | AAA CTA CAC CAA | | | | | | | | | |
| | | ATT CAC TCG CTT | | | | | | | | | |
| | | TTA ATT CCA T | | | | | | | | | |
| mccB | gene intervenant dans la | CGC CTC CAC CAA | 70 | 370- | 70.4 | 47.1 | X57583 | mccB (microcine C51) | 70-mccB370 | | |
| | production de la microcine | CTA ATC CAC CGC | | 301 | | | | | | | |
| | C7 (classe I) | TCC CGT ATC GAG | | | | | | | | | |
| | | CAA TTT TGA CAT | | | | | | | | | |
| | | AGC GAC CCA ATA | | | | | | | | | |
| | | TAT AAT CCA T | | | | | | | | | |
| mcjA | gene structural de la | ATG ATT AAG CAT | 70 | 238- | 65.4 | 28.6 | AF061787 | aucun | 70-mcjA238 | | |
| | microcine J25 (classe I) | TTT CAT TTT AAT | | 169 | | | | | | | |
| | | AAA CTG TCT TCT | | | | | | | | | |
| | | GGT AAA AAA AAT | | | | | | | | | |
| | | AAT GTT CCA TCT | | | | | | | | | |
| | | CCT GCA AAG G | | | | | | | | | |
| eae | intimine (attaching and | AGT TAT TAC CAC | 70 | 937- | 70.6 | 45.7 | U66102 | tous les variants: | 70-eae937 | STJ348 | EHEC, |
| | effacing), autre nom: eaeA, | TCT GCA GAT TAA | | 868 | | | | α2, β2, γ, ε2, κ, λ, ζI, | | O157:H7 | EPEC |
| | ECs4559, z5110, 10025 | CCT CTG CCG TTC | | | | | | η, τ2, ε, θ, β, γ | | (SB22) | |
| | | CAT AAT GTT GTA | | | | | | | | | |
| | | ACC AGG CCT GCA | | | | | | | | | |
| | | ACT GTG ACG A | | | | | | | | | |
| eae | intimine, variant alpha | ACC ACT CTT CGC | 70 | 27578- | 66.8 | 35.7 | AF022236 | proche de alpha2 | 70-eae | | E2348/ |
| (alpha) | | ATC TTG AGC TGT | | 27509 | | | | | (alpha) | | 69 |
| | | TTG TTG TAC CCA | | | | | | | 27578 | | O126:H6 |
| | | TGA AAT TAT AGT | | | | | | | | | (SB28) |
| | | CTG ACT AGA CTT | | | | | | | | | |
| | | ATA ATA TTC A | | | | | | | | | |
| eae | intimine, variant alpha2 | GCA ACT CCA CTG | 70 | 2735- | 68 | 40 | AF530555 | aucun | 70-eae | | |
| (alpha2) | | TTC ATA TCC ACT | | 2666 | | | | | (alpha2) | | |
| | | GTT GTT TGT TGT | | | | | | | 2735 | | |
| | | ACC CAA GAG CTT | | | | | | | | | |
| | | ATA GTC AGA AGA | | | | | | | | | |
| | | GAC TTG TAA T | | | | | | | | | |
| eae | intimine, variant beta | TAG AAA AGG TCA | 70 | 2666- | 69.6 | 47.1 | AF253560 | intimines non | 70-eae | | RDEC- |
| (beta) | | CTT TCT GAT CTA | | 2597 | | | | caractérisées | (beta)2666 | | 1B O15 |
| | | CTA CGG GTG CCC | | | | | | | | | (SB40) |
| | | CCT CCT TCA TCA | | | | | | | | | |
| | | CTC TGA CAG TAT | | | | | | | | | |
| | | AGG TAA TCG C | | | | | | | | | |
| eae | intimine, variant beta2 | TTA TTT TAC ACA | 70 | 2820- | 66 | 28.6 | AF530556 | proche de beta, | 70-eae | | |
| (beta2) | | AAC TGC AAA AGC | | 2751 | | | | intimines non | (beta2)2820 | | |
| | | ATT TTT ATT TTT | | | | | | caractérisées | | | |
| | | TAC TCC CAC ATT | | | | | | | | | |
| | | AGT CAA TTG GTT | | | | | | | | | |
| | | CTT CGT AAC T | | | | | | | | | |
| eae | intimine, variant delta | TTA TTT CAC ACA | 70 | 3093- | 67.5 | 33.4 | U66102 | identique à kappa | 70-eae | DVI-828 | |
| (delta) | | GAC TGC AAA GGC | | 3024 | | | | | (delta) | | |
| | | ATT GTT ATC TGT | | | | | | | 3093 | | |
| | | TGT CTT AAC ATT | | | | | | | | | |
| | | TGT CAG AGA GTT | | | | | | | | | |
| | | TGT TGT GAT T | | | | | | | | | |
| eae | intimine, variant epsilon | ATC CTT TAG CTC | 70 | 2637- | 70 | 44.3 | AY186750 | proche de eta | 70-aea | TB154A | |
| (epsilon) | | ACT CGT AGA TGA | | 2568 | | | | | (epsilon) | | |
| | | CGG CAA GCG TGC | | | | | | | 2637 | | |
| | | ATT ATT CAT TCT | | | | | | | | | |
| | | ACA TGT TGC CTC | | | | | | | | | |
| | | AGC ATC ACT A | | | | | | | | | |
| eae | intimine, variant epsilon2 | AAC GAC CAC TAT | 70 | 2608- | 66.8 | 32.9 | AF530554 | aucun | 70-eae | | |
| (epsilon2) | | TCA TTT CAC ATT | | 2539 | | | | | (epsilon2) | | |
| | | TTG TTT TAG CAA | | | | | | | 2608 | | |
| | | CGT TAT AGA GAA | | | | | | | | | |
| | | CTT TAT CTT GTG | | | | | | | | | |
| | | TTT CCA CAG T | | | | | | | | | |
| eae | intimine, variant eta | TCA CTC GTA GAT | 70 | 2979- | 70.2 | 45.7 | AJ308550 | proche de epsilon | 70-eae(eta) | | |
| (eta) | | GAC GGT AAG CGA | | 2910 | | | | | 2979 | | |
| | | CCA CTA TTC GTG | | | | | | | | | |
| | | CTG CAT GTT GCT | | | | | | | | | |
| | | TCA GCA ACG CTA | | | | | | | | | |
| | | TAG ATT ACT T | | | | | | | | | |
| eae | intimine, variant gamma | TTG TGT AAT CCA | 70 | 3248- | 69.9 | 42.9 | AF253561 | identique à theta | 70-eae | | EDL933 |
| (gamma) | | AGC TGT TAT TGA | | 3179 | | | | | (gamma) | | O157:H7 |
| | | CTG CAT AGA ACG | | | | | | | 3248 | | (SB44) |
| | | ATA ATG GTC ATA | | | | | | | | | |
| | | TCC GTT TGC AGG | | | | | | | | | |
| | | CCC CCA TGA A | | | | | | | | | |
| eae | intimine, variant jota | TTA TCC GTT GCT | 70 | 2481- | 66.9 | 35.7 | AJ308551 | aucun | 70-eae | | |
| (jota) | | ACA GTC TGT AGA | | 2412 | | | | | (jota)2481 | | |
| | | TTC AAT TTA CCT | | | | | | | | | |
| | | AAA TCA GTT GAG | | | | | | | | | |
| | | AAT GTA ACT ACG | | | | | | | | | |
| | | TGT CCC TTT T | | | | | | | | | |
| eae | intimine, variant jota2 | TTG ACT ATC GCT | 70 | 1868- | 68 | 38.6 | AF530553 | aucun | 70-eae | | |
| (jota2) | | TTA CCA GTA TTA | | 1799 | | | | | (jota2)1868 | | |
| | | TCT GTA TTA ACT | | | | | | | | | |
| | | CTT TCA GAG CCA | | | | | | | | | |
| | | AGT TTC CCC ACA | | | | | | | | | |
| | | CCT GAA ACA A | | | | | | | | | |
| eae | intimine, variant lambda | CTA ACA ACA GCT | 70 | 221- | 67.9 | 38.6 | AJ579367 | aucun | 70-eae | | |
| (lambda) | | TTT CCC GCA GCA | | 152 | | | | | (lambda)221 | | |
| | | TTA GAG GTC AAG | | | | | | | | | |
| | | TTT ACA GTT GCA | | | | | | | | | |
| | | TAT CCA TCT TTA | | | | | | | | | |
| | | TCT GTT AAC T | | | | | | | | | |
| eae | intimine, variant mu | GGA CAC ATG CAT | 70 | 2566- | 66.1 | 34.3 | AJ705049 | aucun | 70-eae(mu) | | |
| (mu) | | AAT AAG CTT TTT | | 2496 | | | | | 2566 | | |
| | | GGC CTA CCT TTA | | | | | | | | | |
| | | TCA TAT ATT TTG | | | | | | | | | |
| | | GAG TTT TAA CAG | | | | | | | | | |
| | | TGT AGC TTA C | | | | | | | | | |
| eae | intimine, variant nu | TTT CTC TTA ACC | 70 | 2762- | 69.5 | 42.9 | AJ705050 | proche de zeta | 70-eae(nu) | | |
| (nu) | | AGA TCG TAT GTG | | 2693 | | | | | 2762 | | |
| | | CTT GCA ACG CCC | | | | | | | | | |
| | | TTC TTC ACA TCA | | | | | | | | | |
| | | TCA TCG GTT TGT | | | | | | | | | |
| | | TTT ATC CAC G | | | | | | | | | |
| eae | intimine, variant pi | CAC GTT TTT TCA | 70 | 2588- | 65 | 31.4 | AJ705052 | aucun | 70-eae(pi) | | |
| (pi) | | GCA GAG CTA TAG | | 2519 | | | | | 2588 | | |
| | | ATT TCT GTA TTT | | | | | | | | | |
| | | TGT GAT TCT ACA | | | | | | | | | |
| | | GAT ATT ATC TTA | | | | | | | | | |
| | | TCA GGT GTA T | | | | | | | | | |
| eae | intimine, variant xi | ACT CAT TCG TAG | 70 | 2629- | 67.6 | 40 | AJ705051 | proche de epsilon | 70-eae(xi) | | |
| (xi) | | ATA GCG GTA AAC | | 2560 | | | | et eta | 2629 | | |
| | | GGC CAT TAT TCG | | | | | | | | | |
| | | TTC TAC ATA TTG | | | | | | | | | |
| | | CTT CAG CAT CGT | | | | | | | | | |
| | | TAT AGA CTA C | | | | | | | | | |
| eae | intimine, variant zeta | CTT TGA CAT CAA | 70 | 2233- | 62.9 | 42.9 | AF449417 | aucun | 70-eae | DVI-797 | |
| (zeta) | | TTG CGC TCC CGC | | 2164 | | | | | (zeta)2233 | | |
| | | TAA TAC TAG CGC | | | | | | | | | |
| | | TAA CAA GCG ATT | | | | | | | | | |
| | | TTC CTG TAG TCT | | | | | | | | | |
| | | TCG ATG TTA A | | | | | | | | | |
| eaf | sonde EAF (E. coli | AAC ATC GAT CAG | 70 | 615- | 72.7 | 41.4 | X76137 | aucun | 70-eaf615 | E2348/69 | EPEC |
| | Adherence Factor plasmid) | TGA TTT GGA TCC | | 546 | | | | | | O126:H6 | |
| | | CGT TCG ATC ACT | | | | | | | | (SB28) | |
| | | CCA AGC GTT AAC | | | | | | | | | |
| | | TTA TCA TCT TTC | | | | | | | | | |
| | | TTT TAC CCT G | | | | | | | | | |
| eaf1 | facteur d'adhesion (efa1) et | AAC AAC ATC TTC | 70 | 730- | 69 | 40 | AF159462 | lifA et efa1 (genes | 70-efa | E2348/69 | EHEC, |
| | d'inhibition de la | CAG AGA GTT TTC | | 661 | | | | identiques a 99.9%) | (1)730 | O126:H6 | EPEC |
| | prolifération des | TTT CGA AAC CAT | | | | | | | | (SB28) | |
| | lymphocytes (lifA) des | TTT ATC AAA GAA | | | | | | | | | |
| | EHEC | GCG TAG TCG GGC | | | | | | | | | |
| | | TTC TGA TGC T | | | | | | | | | |
| ehxA | hemolysine, autres noms: | TAT TTC TAT TCC | 70 | 192- | 72.6 | 40 | AF043471 | aucun | 70-ehxA192 | STJ348 | EHEC |
| | EHEC-hlyA | AAG CTC ATC AGC | | 123 | | | | | | O157:H7 | |
| | | AGC TTT GAC CAA | | | | | | | | (SB22) | |
| | | CTC ATT AAT ACC | | | | | | | | | |
| | | CAC GCC CTG AGC | | | | | | | | | |
| | | TTC ATA ATT A | | | | | | | | | |
| espA-1 | proteine secretee EspA, | CCT TAG ATG CCT | 70 | 286- | 68.8 | 42.9 | AF064683 | espA variant β | 70-espA | P86-1390 | EHEC, |
| | groupe I | CAT TCA TAT CAG | | 217 | | | | | (1)286 | (SB42) | EPEC |
| | | CAA ACT TTG CAA | | | | | | | | | |
| | | TCG ACA GAT CGC | | | | | | | | | |
| | | TTT GTG CCT GAT | | | | | | | | | |
| | | ACA TAT AGG C | | | | | | | | | |
| espA-2 | EspA, groupe II | GCG CTT AAA TCA | 70 | 13347- | 70.1 | 37.1 | AF071034 | aucun | 70-espA | EDL933 | EHEC, |
| | | CCA CTA AGA TCA | | 13416 | | | | | (2)13347 | O157:H7 | EPEC |
| | | CGA ATA CCA GTT | | | | | | | | (SB44) | |
| | | ACA CTT ATG TCA | | | | | | | | | |
| | | TTA CGT GGA TCG | | | | | | | | | |
| | | TTT ATA TAG T | | | | | | | | | |
| espA-3 | EspA, groupe III | TGT GCC TCG GTG | 70 | 227- | 73.8 | 44.3 | AJ225016 | aucun | 70-espA | E2348/69 | EHEC, |
| | | GAT TCC TTA GAT | | 158 | | | | | (3)227 | O126:H6 | EPEC |
| | | GAC TCA TTC ATG | | | | | | | | (SB28) | |
| | | TCT GCA TAT GTA | | | | | | | | | |
| | | GCA ATA GAT AGC | | | | | | | | | |
| | | TCG CTT TGT G | | | | | | | | | |
| espB-1 | proteine secretee EspB, | CTG GAA GCG CCG | 70 | 4547- | 73.4 | 44.3 | Y13068 | aucun | 70-espB | EDL933 | EHEC, |
| | group I (autre noms: | GTC GTA CTC TCC | | 4478 | | | | | (1)4547 | O157:H7 | EPEC |
| | z5105, ecs4554) | GAA GCG GAA TTA | | | | | | | | (SB44) | |
| | | ACC ATC GTT ACT | | | | | | | | | |
| | | TGA GTA TTA TCA | | | | | | | | | |
| | | ATA GTA TTC A | | | | | | | | | |
| espB-2 | proteine secretee EspB, | GTA AGT AAA GAT | 70 | 230- | 73.4 | 42.9 | Z21555 | aucun | 70-espB | E2348/69 | EHEC, |
| | groupe II | GAA CTG ATT GAC | | 161 | | | | | (2)230 | O126:H6 | EPEC |
| | | GAA GTT GAT GTA | | | | | | | | (SB28) | |
| | | GTT GTT GAA CTG | | | | | | | | | |
| | | GTG CTG TCA GTC | | | | | | | | | |
| | | GTG CTG CTC A | | | | | | | | | |
| espB-3 | proteine secretee EspB, | CAT TAG AGC CGG | 70 | 73- | 68.3 | 34.3 | X99670 | aucun | 70-espB | P86-1390 | EHEC, |
| | groupe III | TAG TAT TCT CCG | | 04 | | | | | (3)73 | (SB42) | EPEC |
| | | AAA CAG AAT TAA | | | | | | | | | |
| | | CCG TCA TTA CTT | | | | | | | | | |
| | | GAT TAG TAT AAT | | | | | | | | | |
| | | CGA TAG TAT T | | | | | | | | | |
| espC | enterotoxine (EspC) | TGA TAG ATT AAA | 70 | 5434- | 75.4 | 45.7 | AF297061 | aucun | 70-espC5434 | E2348/69 | EPEC |
| | | TAA TGC TAA AAG | | 5365 | | | | | | O126:H6 | |
| | | GCT GCC GCG AGC | | | | | | | | (SB28) | |
| | | GGC TTT CTT CAT | | | | | | | | | |
| | | AAC TCT GGA GGC | | | | | | | | | |
| | | CAG TTC GGA T | | | | | | | | | |
| espP | exoproteine EspP, serine | ATG CAA GTA TGC | 70 | 11365- | 72 | 38.6 | AF074613 | pssa (serìne protease | 70-esp | B83-215 | EHEC, |
| | protease (clive facteur V de | GTT TGT GTT TTT | | 11296 | | | | des STEC) | P11365 | (SB25) | EPEC |
| | coagulation) | TCT TAC CAG TTG | | | | | | | | | |
| | | CTC TTG ATG ATA | | | | | | | | | |
| | | CTC TGC CGG ATA | | | | | | | | | |
| | | ATT CAG AAA C | | | | | | | | | |
| etpD | EtpD (type II secretion | CGA CCA CAG CAA | 70 | 1505- | 73.7 | 42.9 | Y09824 | aucun | 70-etpD1505 | EDL933 | EHEC |
| | pathway) | AAC CAT AAA CGT | | 1436 | | | | | | O157:H7 | |
| | | CCA GCA CAC TGA | | | | | | | | (SB44) | |
| | | GAA AGA ACT GAT | | | | | | | | | |
| | | AAT ATT GTT CCT | | | | | | | | | |
| | | CGT TCA GCA T | | | | | | | | | |
| gafD | adhesine (fimgriae G ou | GTC AGT AAT CTG | 70 | 629- | 69.2 | 42.9 | L33969 | F17a-G, F17b-G, | 70-gafD629 | S5 | commun |
| | F17c ou 20K) | CAC GAT GTT ACT | | 560 | | | | F17c-G, F17d-G | | O15:KRV | |
| | | GTG TCA TTC AGC | | | | | | | | C383 | |
| | | GTA AAT GGA TTC | | | | | | | | (SB12) | |
| | | AGG CTG AAA TTC | | | | | | | | | |
| | | ACT GTG GTC T | | | | | | | | | |
| F17a-A | sous-unite majeure des pili | TCA CGG CAG GCG | 70 | 563- | 72.6 | 50 | AF022140 | aucun | 70-F17aA563 | | |
| | F17a | TAT TGC ACC CTT | | 494 | | | | | | | |
| | | CCA GCA AAA TTG | | | | | | | | | |
| | | TGA AAG GAG TAA | | | | | | | | | |
| | | GTC CCA CCA CTT | | | | | | | | | |
| | | TTC CGC TTG A | | | | | | | | | |
| F17b-A | sous-unite majeure des pili | ATT TAC TTT ATC | 70 | 580- | 67.6 | 38.6 | L14318 | aucun | 70-F17bA580 | S5 | ETEC |
| | F17b | AAC TCC TGA TGC | | 511 | | | | | | O15:KRV | |
| | | GGC AGA AAC TGT | | | | | | | | C383 | |
| | | ACA TCC CGT TAG | | | | | | | | (SB12) | |
| | | TTG AAT AGT AAA | | | | | | | | | |
| | | TGG TGT AAG G | | | | | | | | | |
| F17c-A | sous-unite majeure des pili | TAG CGG CAG CAG | 70 | 271- | 72.3 | 51.4 | L43373 | aucun | 70-F17cA271 | | |
| | F17c (autres noms: 20K et G) | TAT TAC ACC CAC | | 202 | | | | | | | |
| | | TCA GTG AAA TTG | | | | | | | | | |
| | | TGA AAG GAG TAA | | | | | | | | | |
| | | GGC CTG CTA CCT | | | | | | | | | |
| | | TCC CGG GTG A | | | | | | | | | |
| F17d-A | sous-unite majeure des pili | GAT CTG AAC ATT | 70 | 831- | 67.4 | 38.6 | L77091 | aucun | 70-F17dA831 | | |
| | F17d (autre nom: F111) | TGT TGC ATT ACC | | 762 | | | | | | | |
| | | AGA GCC GCT TGC | | | | | | | | | |
| | | AAT ATT AAG GTT | | | | | | | | | |
| | | ATG ACT ATC ATA | | | | | | | | | |
| | | ATC AGT GGT C | | | | | | | | | |
| fedA | sous-unite fimbriale majeure | GGA AGT CAC CCG | 70 | 637- | 71.2 | 47.1 | M61713 | variants F18ab et | 70-fedA637 | P88-1199 | ETEC |
| | (pili F107 ou F18) | GGG TTT GAC CAC | | 568 | | | | F18ac | | O139:K82 | |
| | | CTT TCA GTT GGG | | | | | | | | (SB6) | |
| | | CAG TAA ATT TGA | | | | | | | | | |
| | | AAC CTT CCG TAG | | | | | | | | | |
| | | TTG CTT TTG A | | | | | | | | | |
| fedAab | variants F18ab | CGC CTT AAC CTC | 70 | 540- | 71.5 | 50 | M61713 | variants F18ac | 70-fed | | |
| | | CTG CCC CTG TGT | | 471 | | | | (6 et 8 differences | Aab540 | | |
| | | TTT ACC GTT CAC | | | | | | dispersees) | | | |
| | | GGT TTT CAG AGC | | | | | | | | | |
| | | GAC ATA TGA ATC | | | | | | | | | |
| | | ATT TGC CAC C | | | | | | | | | |
| fedAac | variants F18ac | CTT AAC CTC CTG | 70 | 318- | 71.4 | 48.6 | L26105 | variants F18ab | 70-fed | | |
| | | CGC CGG CTG TGT | | 249 | | | | (8 differences | Aac318 | | |
| | | TTT ACC GTT CAC | | | | | | dispersees) | | | |
| | | GGT TTT CAG AGC | | | | | | | | | |
| | | AAC ATA TGA ATC | | | | | | | | | |
| | | TCT TGC CAC T | | | | | | | | | |
| faeG | sous-unite fimbriale majeure | CAA AAT TGG CTT | 70 | 281- | 68.2 | 38.6 | M29374 | variants K88 (ab1 | 70-faeG281 | P97- | ETEC |
| | (pili K88 variant ab) | ATT ACC AGT AAC | | 212 | | | | et 2, ac, ad) | | 2554B | |
| | | AGT AAT GGT CAG | | | | | | | | O149:K91 | |
| | | TTT GGT TCC ACC | | | | | | | | (SB9) | |
| | | ATT GGT CAG GTC | | | | | | | | | |
| | | ATT CAA TAC A | | | | | | | | | |
| faeGab | variant ab | CTG TGC GCG CCG | 70 | 642- | 75.9 | 62.9 | M29374 | variant K88ad (9 | 70-fae | K12 | |
| | | CTG CGG CAC TCC | | 573 | | | | differences dispersees | Gab642 | K88ab | |
| | | CAC TCG TGA GTG | | | | | | au centre) | | (SB2) | |
| | | CAG CAC CCG AAA | | | | | | | | | |
| | | CAT TCG TCG TCA | | | | | | | | | |
| | | AAC CAC CAT A | | | | | | | | | |
| faeGac | variant ac | GCT GCG GCA CTC | 70 | 625- | 73.9 | 57.1 | U19784 | aucun | 70-fae | K12 | |
| | | CCA GCC GAG AGT | | 556 | | | | | Gac625 | K88ac | |
| | | TCA GAA CCC CTC | | | | | | | | (SB8) | |
| | | GGC AAA CCA CCA | | | | | | | | | |
| | | TAA AAG ATA GAG | | | | | | | | | |
| | | CTC AAC CCG T | | | | | | | | | |
| faeGad | variant ad | CTG TGC GCG CCG | 70 | 642- | 73.9 | 55.7 | M29376 | variant K88ab (10 | 70-fae | K88ad | |
| | | CTG CGG CAC TCC | | 573 | | | | differences dispersees | Gad642 | (SB7) | |
| | | CAC CCT TGA GTT | | | | | | au centre), K88ac | | | |
| | | CAG AAT TCT TAA | | | | | | (36/70) | | | |
| | | CAT TCG TCG GCA | | | | | | | | | |
| | | AAC CAC CAT A | | | | | | | | | |
| fanC | sous-unite fimbriale majeure | ATT ACC ATT GAC | 70 | 210- | 71.2 | 37.1 | M35282 | aucun | 70-K(99)210 | B44s | ETEC |
| | (pili K99) | CTC AGG GTC AAT | | 141 | | | | | | O9:K30 | |
| | | TGT ACA AGT AGC | | | | | | | | (SB15) | |
| | | ACT CGT TAT TTT | | | | | | | | | |
| | | GCC ATT GAA GTT | | | | | | | | | |
| | | AAT AGT ACC T | | | | | | | | | |
| FimF41A | sous-unite fimbriale majeure | ATG TCA CCT GGT | 70 | 352- | 79.7 | 54.3 | X14354 | aucun | 70-fim | B44s | ETEC |
| | (fimbriae F41) | TGA CCT TCC GTC | | 283 | | | | | F41a352 | O9:K30 | |
| | | CAA TCA GCA GCC | | | | | | | | (SB15) | |
| | | ATC ACT GAA CCA | | | | | | | | | |
| | | GAT ACT GCC GCT | | | | | | | | | |
| | | GAT GCA GCC A | | | | | | | | | |
| fasA | sous-unite fimbriale majeure | CTG CGA GCG AGT | 70 | 328- | 73.7 | 42.9 | M35257 | aucun | 70-fasA328 | P81-603A | ETEC |
| | (fimbriae 987P ou F6), | AAC CAC TGA ACA | | 259 | | | | | | O9:K- | |
| | autre nom: fapC | GAG AGG AAA GCA | | | | | | | | (SB5) | |
| | | CTG CTA ATG TTA | | | | | | | | | |
| | | ATG CGG ATT TTT | | | | | | | | | |
| | | TCA TTC TCA T | | | | | | | | | |
| fimA | ssu majeure des fimbriae de | TGA TCA ACA GAG | 70 | 3145- | 72.6 | 51.4 | Z37500 | tous les variants fimA | 70-fimA3145 | B79-3292 | commun |
| | type 1 (ou F1), parfois | CCT GCA TCA ACT | | 3076 | | | | | | (SB24) | |
| | appelé pilA | GCG CAA GCG GCG | | | | | | | | | |
| | | TTA ACA ACT TCC | | | | | | | | | |
| | | CCT TTA AAG TGA | | | | | | | | | |
| | | ACG GTC CCA C | | | | | | | | | |
| fimH | adhésine des fimbriae de | AGG CGA ATG ACC | 70 | 1409- | 77 | 48.6 | AJ225176 | aucun | 70-fimH1409 | B79-3292 | commun |
| | type 1 | AGG CAT TTA CCG | | 1340 | | | | | | (SB24) | |
| | | ACC AGC CCA TCA | | | | | | | | | |
| | | GCA GTA CAG CAA | | | | | | | | | |
| | | ACA GGG TAA TAA | | | | | | | | | |
| | | CTC GTT TCA T | | | | | | | | | |
| f165(1)A | ssu majeure des fimbriae | ACC GCC GTT AGT | 70 | 1532- | 70.9 | 45.7 | L07420 | quelques variants | 70-f165(1) | | |
| | F165(1) (pili Prs-like), | TGC TAA TTC TTC | | 1463 | | | | de papA | A1532 | | |
| | autre nom: fooA | AGC CTG CCC CGT | | | | | | | | | |
| | | TAC TTG TGG CCC | | | | | | | | | |
| | | AGT AAA AGA TAA | | | | | | | | | |
| | | TTG AAC CTT A | | | | | | | | | |
| fliC | sous unité flagellaire | CAG ACT GGT TCT | 70 | 70- | 72.9 | 41.4 | U47614 | aucun | 70-fliC70 | E32511 | commun |
| | majeure (flagelline), autres | TGT TGA GAT TAT | | 1 | | | | | | O157:H7 | |
| | noms: hag, flaf | TTT GAG TGA TCA | | | | | | | | (SB4) | |
| | | GCG AGA GGC TGT | | | | | | | | | |
| | | TGG TAT TAA TGA | | | | | | | | | |
| | | CTT GTG CCA T | | | | | | | | | |
| flmA | sous unité flagellaire | AAG ACT GAG ATT | 70 | 7238- | 69.9 | 42.9 | AB128918 | flkA3, flkA53 | 70-flmA7238 | | commun |
| | majeure (flagelline), | TGT TCA GGT TGT | | 7169 | | | | (variants de fliC), | | | |
| | variant de fliC | TCT GAG TCA ACA | | | | | | fliC | | | |
| | | GCG ACA GGC TGT | | | | | | | | | |
| | | TGG TAT TGA TAA | | | | | | | | | |
| | | CTT GTG CCA T | | | | | | | | | |
| sfaA | sous unité majeure des | TAT TCT GTA GAG | 70 | 17300- | 68.2 | 38.6 | X16664 | aucun | 70-sfa | P81-4787 | UPEC |
| | fimbriae de type S (Sfal) | ACA GCA CAT CAT | | 17231 | | | | | A17300 | O115:KV | |
| | | TGT GTG TAG CAA | | | | | | | | 165 | |
| | | TAA CAT TTC CTG | | | | | | | | (SB23) | |
| | | CAA AGA TAA TTG | | | | | | | | | |
| | | ATG CAT GCC C | | | | | | | | | |
| sfaHII | ssu mineure des fimbriae de | GAC ACC ATA TTG | 70 | 1510- | 70.1 | 45.7 | S53210 | sfaH (pili Sfal) | 70-sfa | | MENEC |
| | type S(SfaII) | ATA AAA CGC CTC | | 1441 | | | | | HII1510 | | |
| | | TGT CAC CTG CAA | | | | | | | | | |
| | | ATC AAA CTG AAG | | | | | | | | | |
| | | TGG TAA TTG CCT | | | | | | | | | |
| | | GGC ATA CCC C | | | | | | | | | |
| facA | ssu majeure des fimbriae | AGC TTT GTA TAG | 70 | 586- | 69.5 | 41.4 | X76121 | sfaA11 (fimbriae SfaII) | 70-facA586 | | APEC |
| | AC/I | CCA AGG CGT TAT | | 517 | | | | | | | |
| | | TTT TTC CAG CAA | | | | | | | | | |
| | | CAG GTG TGC CAG | | | | | | | | | |
| | | AAA AAA GAA TCT | | | | | | | | | |
| | | TCA CAG ATC C | | | | | | | | | |
| focA | ssu fimbriale majeure des | GTT AAT GTA AAC | 70 | 611- | 68.8 | 41.4 | AF298200 | f165(2)A | 70-focA611 | CFT073 | UPEC |
| | fimbriae FIC | GTT GAG CTT GCA | | 542 | | | | (F1C-like) | | | |
| | | GTT CCA TCT AAA | | | | | | | | | |
| | | GGT ACA ACC TTG | | | | | | | | | |
| | | CCG GTA TGG TCA | | | | | | | | | |
| | | GTA ATC TGA A | | | | | | | | | |
| fepC | ferric enterobactin transport | TAT TGC CTG GGT | 70 | 10105- | 73.8 | 54.3 | AF081283 | aucun | 70-fep | EDL933 | UPEC, |
| | ATP-binding protein | GCC GCA GGC GCA | | 10036 | | | | | C10105 | O157:H7 | |
| | | CGA CGG CAT TTT | | | | | | | | (SB44) | |
| | | TGG TTT TAG TGT | | | | | | | | | |
| | | GCT GGA TAT GGT | | | | | | | | | |
| | | GTT GAT GGG G | | | | | | | | | |
| fyuA | gène du recepteur de la | GTT GGC TGA TGC | 70 | 302- | 79.4 | 54.3 | Z38064 | aucun | 70-fyuA302 | P84-1195 | UPEC |
| | pesticine et de la | CGA GCG GGA AGA | | 233 | | | | | | O9:K28 | |
| | yersinlabactine | TTG TTT ACT GGC | | | | | | | | (SB26) | |
| | | GGT AAC CAC CAG | | | | | | | | | |
| | | CGT GCT TTC GTC | | | | | | | | | |
| | | TTG CTG TGA A | | | | | | | | | |
| hra1 | adhesine non fimbriale- | GTG ACA ACG ATT | 70 | 617- | 71.3 | 51.4 | U07174 | hek (adhesine | 70-hra | | commun |
| | hemagglutinine | CGC GAC CAC TGC | | 548 | | | | similaire a hral) | (1)617 | | |
| | | TTC CGT ACC CAT | | | | | | | | | |
| | | AAT CCC AGG TAC | | | | | | | | | |
| | | TGA TAC CGG TTG | | | | | | | | | |
| | | TTT TCT GGT G | | | | | | | | | |
| hlyA | hémolysine A | ATT TAT TTG CAG | 70 | 1389- | 75.1 | 41.4 | M10133 | hlyA plasmide | 70-hlyA1389 | J96 | UPEC |
| | (chromosome), ssu | CGG ATT GCT TTG | | 1320 | | | | | | O4:K12 | |
| | structurale | CAG ACT GCA GTG | | | | | | | | (SB18) | |
| | | TGC TTT TAA TTT | | | | | | | | | |
| | | GTG CAG CGG TTA | | | | | | | | | |
| | | TTG TTG GCA T | | | | | | | | | |
| hlyE | hemolysine E, autres noms: | TTT GGC GGC ATC | 70 | 867- | 69.6 | 41.4 | U57430 | sheA, hrp, clyA | 70-hlyE867 | EDL933 | commun |
| | sheA, hrp, clyA | GAT ATC TTT ATT | | 798 | | | | | | O157:H7 | |
| | | CGC TTG TTT AAC | | | | | | | | (SB44) | |
| | | CGT GTT AGA CAG | | | | | | | | | |
| | | GGT GGT AAA GAA | | | | | | | | | |
| | | ATT CTG CAC A | | | | | | | | | |
| aucun | hemolysine E des souches | TGT GGA TGC CGA | 70 | 248- | 66.8 | 35.7 | AF052225 | aucun | 70-hlyE | | APEC |
| hlyE | aviaires | TTG AGA GTA CTC | | 179 | | | | | (a)248 | | |
| | | TTC TTT AAA ACG | | | | | | | | | |
| | | GCT TAA TTC TTT | | | | | | | | | |
| | | CAC TGT ATC GTT | | | | | | | | | |
| | | AAA TGT ATT C | | | | | | | | | |
| ibeA | protéine d'invasion | CAC CAA CAA CTA | 70 | 17545- | 74.1 | 44.3 | AF289032 | aucun | 70-ibe | H87-5480 | MENEC |
| | | ACA CTT CCG TGG | | 17476 | | | | | A17545 | O18 | |
| | | TTG CCA GTA CAG | | | | | | | | (SB36) | |
| | | GTA TAT TAC GAG | | | | | | | | | |
| | | CGG GTT CCA GAT | | | | | | | | | |
| | | AAA ATT CCA T | | | | | | | | | |
| ibeB | proteine d'invasion des | CGC CGG TAA TTT | 70 | 893- | 74.1 | 55.7 | AF094824 | aucun | 70-ibeB893 | RS 218 | commun |
| | bmec (systeme d'efflux des | AAC GCT TTG CAG | | 824 | | | | | | O18:K1:H7 | |
| | cations), autres noms: ylcB, | GCT GTC GCT GTT | | | | | | | | | |
| | cusC | TAC TGT CTG CGC | | | | | | | | | |
| | | TTG CGG CAG CTT | | | | | | | | | |
| | | GCC GTA GCT T | | | | | | | | | |
| iha | nouvelle protéine d'adhesion | CAG CAG CTA TGC | 70 | 3105- | 77.6 | 51.4 | AF126104 | aucun | 70-iha3105 | E32511 | commun |
| | | TGC TGG CTG AAA | | 3036 | | | | | | O157:H7 | |
| | | ATC CGA GAC AGG | | | | | | | | (SB4) | |
| | | GAA TGA CTA CGG | | | | | | | | | |
| | | AAG CCA GAG TGG | | | | | | | | | |
| | | TTA TTC GCA T | | | | | | | | | |
| invX | protéine d'invasion | CTA CTG GCC ATA | 70 | 94- | 67.3 | 32.9 | L18946 | aucun | 70-invX94 | H84 | EIEC |
| | | AGG AAA AGA TAA | | 25 | | | | | | (SB49) | |
| | | GGA TTA AAT AAA | | | | | | | | | |
| | | GAG CCT TAT TAC | | | | | | | | | |
| | | CCA TAT AAA CTA | | | | | | | | | |
| | | TAT CAG ACA C | | | | | | | | | |
| ipaB | protéine d'invasion | ACA CTA ACG ATA | 70 | 968- | 68.3 | 40.0 | AY098990 | aucun | 70-ipaB968 | E32511 | EIEC |
| | (invasion plasmid antigen | GTT AAA AGT GCC | | 899 | | | | | | O157:H7 | |
| | B) | CCA AGT ATT TTC | | | | | | | | (SB4) | |
| | | CCA ACA CAA CCC | | | | | | | | | |
| | | ATT ACT CTG TTG | | | | | | | | | |
| | | AGT TCT TCT G | | | | | | | | | |
| iroN | récepteur sidérophore | CTA CTG ATA CCT | 70 | 390- | 73 | 42.9 | AF135597 | aucun | 70-iroN390 | CP9 | UPEC, |
| | | GGC TAT TCA ACC | | 321 | | | | | | (SB50) | APEC |
| | | CAA CTA GGA GCA | | | | | | | | | |
| | | CAG TTA GCG ACC | | | | | | | | | |
| | | AGA GGA TTT TGT | | | | | | | | | |
| | | TAA TTC TCA T | | | | | | | | | |
| irp1 | protéine de biosynthèse de | TTC GCC ATC CGG | 70 | 124301- | 74.8 | 60.0 | AE016762 | aucun | 70-irp(1) | P84-1195 | UPEC |
| | la yersiniabactine | CGA TTC AGG AAA | | 124232 | | | | | 124301 | O9:K28 | |
| | (peptide/polyketide | ATG GCA GGC GTA | | | | | | | | (SB26) | |
| | synthétase) | GCC GAT AAC CGC | | | | | | | | | |
| | | GAC AGG TTC GCA | | | | | | | | | |
| | | GTC CGG GTA G | | | | | | | | | |
| irp2 | peptide synthétase supposée | CAT TGG GTG GCG | 70 | 117764- | 75.9 | 61.4 | AE016762 | aucun | 70-irp(2) | P84-1195 | UPEC |
| | (ligase),(impliquee dans | TTG CAG CAA GGT | | 117695 | | | | | 117764 | O9:K28 | |
| | l'acquisition de fer) | CGT GAT GGC CTG | | | | | | | | (SB26) | |
| | | CTC CAG CTG CGA | | | | | | | | | |
| | | CGC CGT CAG ACA | | | | | | | | | |
| | | ATG GCC TTC A | | | | | | | | | |
| iss | serum survivance and | GAG CAC ATC CTG | 70 | 361- | 68.0 | 37.1 | AF042279 | ybcU (homologue | 70-iss361 | B79-3292 | commun |
| | surface exclusion protein | TAA TAA GCA TTG | | 292 | | | | de bor) | | (SB24) | |
| | (homologue de Bor du | CCA GAG CGG CAG | | | | | | | | | |
| | phage lamda) | AAA ATA ACA TTT | | | | | | | | | |
| | | TTT TCA TCT TAT | | | | | | | | | |
| | | TAT CCT GCA T | | | | | | | | | |
| iucD | N-6-hydroxylysine (L-lysine- | TAG GGA TTT GTA | 70 | 319- | 75 | 47.5 | M18968 | aucun | 70-iucD319 | P81-4787 | commun |
| | 6-monooxygenase), autre | GGT GCA ACA GCA | | 250 | | | | | | O115:KV | |
| | nom: aerA (operon | CTG ACC AGA TCT | | | | | | | | 165 | |
| | aerobactine) | TTC AGA AAG ACG | | | | | | | | (SB23) | |
| | | GTC TGC ATA TGA | | | | | | | | | |
| | | CAA TCC GGT A | | | | | | | | | |
| iutA | récepteur de la cloacine | CTG CTG GCG CCA | 70 | 238- | 76 | 47.1 | X05874 | aucun | 70-iutA238 | P81-4787 | UPEC, |
| | DF13 (aerobactine), ancien | TCA TGG TAA GAA | | 169 | | | | | | O115:KV | APEC |
| | nom DF13 | GCA GTG GGT TGA | | | | | | | | 165 | |
| | | GAG CCC AAA GCG | | | | | | | | (SB23) | |
| | | TAT ACT TTT TGC | | | | | | | | | |
| | | TTA TCA TCA T | | | | | | | | | |
| katP | catalase/peroxidase des | TCT TTT TTA TCA | 70 | 213- | 75.9 | 48.6 | X89017 | aucun | 70-katP213 | EDL933 | EHEC |
| | EHEC | GCG GCT ACA GCG | | 144 | | | | | | O157:H7 | |
| | | GTA GAA AAG CTC | | | | | | | | (SB44) | |
| | | CCC GAT AGC GCC | | | | | | | | | |
| | | AGA AGA ATC AGA | | | | | | | | | |
| | | ACA GGA AGA G | | | | | | | | | |
| kpsMII | protéine de transport de | AAG ATA AAA AAG | 70 | 406- | 70.7 | 44.3 | X53819 | aucun | 70-kpsM | K5 (F9) | ExPEC |
| | l'acide polysialique, groupe | GGA ATC AGG CCA | | 337 | | | | | (II)406 | 3669 | |
| | II (K1, K4, K5, K7, K12, | TTA AGT AAA AAC | | | | | | | | SB(45) | |
| | K30, K42, K92) | ACC GGG AAT GAG | | | | | | | | | |
| | | ATG TCT GGC ATC | | | | | | | | | |
| | | GTG CGG TGC A | | | | | | | | | |
| kpsMIII | protéine de transport de | AGC CAA ATA CTA | 70 | 3526- | 72.2 | 38.6 | AF007777 | aucun | 70-kpsM | B83-215 | ExPEC |
| | l'acide polysialique, groupe | CAT CAC GTA ATA | | 3457 | | | | | (III)3526 | (SB25) | |
| | III (K2, K3, K10, K11, K19, | CTT GCA AAG AAG | | | | | | | | | |
| | K54) | TGC GTG GAG TTT | | | | | | | | | |
| | | GAC TAA TAA TGG | | | | | | | | | |
| | | GTT TGT CCA T | | | | | | | | | |
| kfiB | proteine impliquee dans la | TTG AAA GAA ATT | 70 | 5929- | 68.4 | 31.4 | X77617 | aucun | 70-kfiB5925 | K5 (F9) | ExPEC |
| | biosynthese de la capsule | GGC ATG AAC TCA | | 5860 | | | | | | 3669 | |
| | K5 | CCA AAT TAT TCT | | | | | | | | SB(45) | |
| | | ACA AGT AAT AAA | | | | | | | | | |
| | | ATT TCC CCA GAA | | | | | | | | | |
| | | TAT ATC ACC G | | | | | | | | | |
| neuA | N-acétylneuraminique acide | CAT TTC TGA CTG | 70 | 155- | 71.4 | 37.1 | J05023 | aucun | 70-neuA155 | | ExPEC |
| | synthétase (antigène K1) | CAA GGC AGC TTC | | 86 | | | | | | | |
| | | AAT TGT ATA AGC | | | | | | | | | |
| | | AAG AAG AGG TTT | | | | | | | | | |
| | | ATC TAT CAG CAT | | | | | | | | | |
| | | CAA AGC ATT T | | | | | | | | | |
| neuC | protéine p7 (impliquée dans | ATT TCC ATA CGC | 70 | 291- | 71.8 | 37.1 | M84026 | aucun | 70-neuC291 | U9/41 | MENEC |
| | la synthèse d'acide | ATT ATC ACA ATG | | 222 | | | | | | O2:K1 | |
| | polysialic) | CAT TCC TGT AAC | | | | | | | | SB(46) | |
| | | TGC CAA ATC AAG | | | | | | | | | |
| | | CTG TAT TTC TGG | | | | | | | | | |
| | | AGT TTC TCT T | | | | | | | | | |
| L7095 | cytotoxine supposée (aussi | GGC CAT GTT TAA | 70 | 78623- | 67.8 | 31.4 | AF074613 | aucun | 70-L(7095) | EDL933 | EHEC |
| | appelée toxine B (gène: | CAT CAG TAC TAA | | 78554 | | | | | 78623 | O157:H7 | |
| | toxB) rien à voir avec | CAT TTT TAA CTC | | | | | | | | (SB44) | |
| | enterotoxine B) | TTG TAT TGT TAA | | | | | | | | | |
| | | TTG CTT TAT CTA | | | | | | | | | |
| | | AAG AAG AGC C | | | | | | | | | |
| leoA | indispensable pour | ATT TCT AAC ATT | 70 | 80- | 72.5 | 38.6 | AF170971 | aucun | 70-leoA80 | P97- | ETEC |
| | l'exportation d'enterotoxine | CCG CGC AAC TGT | | 11 | | | | | | 2554B | |
| | heat-labile d'ETEC | AAT AGC GAG TTA | | | | | | | | O149:K91 | |
| | | ATC GCA GCC TGT | | | | | | | | (SB9) | |
| | | TTT TCA ATA CTG | | | | | | | | | |
| | | AAC TGT TTG A | | | | | | | | | |
| lpfA | lpfA (long polar fimbriae) | CCC AGA ACA ACT | 70 | 510- | 71.4 | 48.6 | AY156523 | aucun | 70-lpfA510 | | REPEC |
| | des repec | TCT TGT TTT TGA | | 441 | | | | | | | |
| | | GTG TCT GGA GAC | | | | | | | | | |
| | | ACA ACA CAA GCG | | | | | | | | | |
| | | GCG TCA ACA ATC | | | | | | | | | |
| | | TCA CCG GTG A | | | | | | | | | |
| lpfA | lpfA des ehec (O157) | TTA CAG GCG AGA | 70 | 7913- | 71.7 | 51.4 | AE005581 | aucun | 70-lpfA | EDL933 | EHEC |
| (O157) | | TCG TGG ATT CAC | | 7844 | | | | | (O157)7913 | O157:H7 | |
| | | CTT GCG TAC TGT | | | | | | | | (SB44) | |
| | | CCG TTG ACT CTC | | | | | | | | | |
| | | AGA ACC AGG AAG | | | | | | | | | |
| | | TTG TGT TGG G | | | | | | | | | |
| lpfA | lpfA des ehec (O113) | TCG GCT GTA TCG | 70 | 370- | 70.3 | 45.7 | AY057066 | aucun | 70-lpfA | | EPEC, |
| (O113) | | GAG GTA ACT TCA | | 301 | | | | | (O113)370 | | EHEC |
| | | CAA GTA GTG TCG | | | | | | | | | |
| | | ACA ATT TCA CCG | | | | | | | | | |
| | | ACG AAG TGA ACA | | | | | | | | | |
| | | ACA CCA TCT T | | | | | | | | | |
| IngA | sous-unité fimbriale majeure | AGA ATC ACG ACA | 70 | 212- | 75.6 | 47.1 | AF004306 | aucun | 70-lngA212 | PB-176P | ETEC |
| | des pili longus (type IV) | CCG GCT GCA ATC | | 143 | | | | | | (SB30) | |
| | | GTA CCG ATA ATG | | | | | | | | | |
| | | CCA AGA ACA ATG | | | | | | | | | |
| | | ATA ACT TCC AGC | | | | | | | | | |
| | | AGG CTC ATA C | | | | | | | | | |
| toxA | heat-labile enterotoxine (LT | CTG AGA TAT ATT | 70 | 120- | 66.8 | 37.1 | J01646 | aucun | 70-toxA120 | P97- | ETEC |
| | ou LTh), sous-unité A, | GTG CTC AGA TTC | | 51 | | | | | | 2554B | |
| | autres noms: eltA, ltpA, | TGG GTC TCC TCA | | | | | | | | O149:K91 | |
| | lthA | TTA CAA GTA TCA | | | | | | | | (SB9) | |
| | | CCT GTA ATT GTT | | | | | | | | | |
| | | CTT GAT GAA T | | | | | | | | | |
| toxB | heat-labile enterotoxine (LT | GGG GAG CTC CGT | 70 | 274- | 70.4 | 37.1 | J01646 | aucun | 70-toxB274 | P97- | ETEC |
| | ou LTh), sous-unité G, | ATG CAC ATA GAG | | 205 | | | | | | 2554B | |
| | autres noms: eltB, ltpB, | AGG ATA GTA ACG | | | | | | | | O149:K91 | |
| | lthB | CCG TAA ATA AAA | | | | | | | | (SB9) | |
| | | CAT AAC ATT TTA | | | | | | | | | |
| | | CTT TAT TCA T | | | | | | | | | |
| LT-IIaA | heat-labile enterotoxine de | TTC ATC AGG TGT | 70 | 152- | 69.8 | 32.9 | M17894 | aucun | 70-ltIIa | | ETEC |
| | type IIa (sous-unité A) | TCT GGA GTC TGC | | 83 | | | | | A152 | | |
| | | TCT AAA GAA ATC | | | | | | | | | |
| | | GTT TGC TGA AAC | | | | | | | | | |
| | | AGA AAA TGA TAT | | | | | | | | | |
| | | AAA AAC AAA A | | | | | | | | | |
| LT-IIaB | heat-labile enterotoxine de | CAG CAT ATA CCT | 70 | 898- | 72.2 | 40 | M17894 | aucun | 70-ltIIa | | ETEC |
| | type IIa (sous-unité B) | GAC CAG ACA GAA | | 829 | | | | | B898 | | |
| | | TGC CAG TCA TCA | | | | | | | | | |
| | | GAA CAA AAG CAC | | | | | | | | | |
| | | CAA TTA TTT TCT | | | | | | | | | |
| | | TAG AGC TCA T | | | | | | | | | |
| LT-IIbA | heat-labile enterotoxine de | GGC GTT CTC GAA | 70 | 204- | 68.2 | 31.4 | M28523 | aucun | 70-ltIIb | | ETEC |
| | type IIb (sous-unité A) | TCA GCC CTG AAA | | 135 | | | | | A204 | | |
| | | TAA TCA TTT GCA | | | | | | | | | |
| | | TAT AAA GGA AAG | | | | | | | | | |
| | | GAT ATT AGA AAT | | | | | | | | | |
| | | AAA GAA ATA A | | | | | | | | | |
| LT-IIbB | heat-labile enterotoxine de | CTG CAT GTG CCT | 70 | 963- | 72.3 | 38.6 | M28523 | aucun | 70-ltIIb | | ETEC |
| | type IIb (sous-unité B) | GAA CAG ATA CCA | | 894 | | | | | B963 | | |
| | | AAG CAG CCA TGA | | | | | | | | | |
| | | TAA CAA ATG CCT | | | | | | | | | |
| | | TGA TAA TTT TCT | | | | | | | | | |
| | | TAA AGC TCA T | | | | | | | | | |
| ompA | protéine de membrane | AGT ATC ATG GTA | 70 | 1162- | 78.7 | 54.3 | V00307 | aucun | 70-ompA1162 | J96 | commun |
| | externe OMPA (ou OMPII), | CTG GGA CCA GCC | | 1096 | | | | | | O4:K12 | |
| | autres noms: tolG, tut, con | CAG TTT AGC ACC | | | | | | | | (SB18) | |
| | | AGT GTA CCA GGT | | | | | | | | | |
| | | GTT ATC TTT CGG | | | | | | | | | |
| | | AGC GGC CTG C | | | | | | | | | |
| ompT | protéine de membrane | TCT CGG TAG AAG | 70 | 529- | 77.3 | 50 | X06903 | aucun | 70-ompT529 | J96 | commun |
| | externe 3b ou protéase VII | CAA AAG AGC TGA | | 460 | | | | | | O4:K12 | |
| | (également appelée: omptin | TCG CAA TAG GGG | | | | | | | | (SB18) | |
| | ou protéase a) | TTG TCA GGA CTA | | | | | | | | | |
| | | TTC CCA GAA GTT | | | | | | | | | |
| | | TCG CCC GCA T | | | | | | | | | |
| paa | proteine associée aux effets | CAT ACA GAT TGA | 70 | 70- | 70.2 | 35.7 | U82533 | aucun | 70-paa70 | STJ348 | EHEC, |
| | d'attachement/effacement | TAT CAG CAT AAG | | 1 | | | | | | O157:H7 | EPEC |
| | chez le porc (facteur de | CAG CAG AAG ACA | | | | | | | | (SB22) | |
| | colonisation intestinal) | GGA ATA TTA AAA | | | | | | | | | |
| | | AAC CTG CCA TTA | | | | | | | | | |
| | | TGT TCC TCA T | | | | | | | | | |
| papGI | adhésine des pili P (allele I) | AGG GTA TAT ATA | 70 | 8838- | 65.9 | 32.9 | X61239 | aucun | 70-pap | J96 | UPEC |
| | | GCT GAG GTT GGT | | 8769 | | | | | GI8838 | O4:K12 | |
| | | CAA TAA CCT TAA | | | | | | | | (SB18) | |
| | | CAT TAC CAG CAT | | | | | | | | | |
| | | TTG TAG TTA AAT | | | | | | | | | |
| | | AGT CGT TAA A | | | | | | | | | |
| papGI2 | adhésine des pili P | AGT GGA TGG AAA | 70 | 160- | 71 | 45.7 | AF247505 | aucun | 70-pap | | UPEC |
| | (allèle I-2) | ACT GCG GTT TAT | | 91 | | | | | GI2160 | | |
| | | CAA CGA CCT TAA | | | | | | | | | |
| | | CCT GAC CCG CAT | | | | | | | | | |
| | | TAT GGC TGG AAT | | | | | | | | | |
| | | GGT CGT TAA A | | | | | | | | | |
| papGII | adhésine des pili P | ATG CCC GGG CGC | 70 | 1391- | 71.8 | 48.6 | M20181 | aucun | 70-pap | IA2 | UPEC, |
| | (allèle II) | CAC GAA GTT ATA | | 1322 | | | | | GII1391 | (SB43) | APEC |
| | | AAT TGT GGC CTT | | | | | | | | | |
| | | TGA GTA ATC ACC | | | | | | | | | |
| | | ACA TTC CCT CCC | | | | | | | | | |
| | | TGA TAA GAG T | | | | | | | | | |
| papGIII | adhésine des pili P (allèle | ACG GCA TCC TCC | 70 | 651- | 66.7 | 31.4 | AF237473 | fl65(1)G, prfG | 70-pap | CP9 | UPEC, |
| | III), autre nom: prsG | GGT ATT TTT AAT | | 582 | | | | | GIII651 | (SB50) | APEC |
| | | TGA GAA ATT CAA | | | | | | | | | |
| | | TGT ACC ATT AAA | | | | | | | | | |
| | | AGG AAA TGT TTT | | | | | | | | | |
| | | CAT TAA CGA A | | | | | | | | | |
| papGIV | adhésine des pili P | ATG GAA TAG TGA | 70 | 160- | 66 | 31.4 | AF304159 | aucun | 70-pap | | UPEC |
| | (allèle IV) | ATT GTC CCC TGT | | 91 | | | | | GIV160 | | |
| | | CAA AAA TTG TCA | | | | | | | | | |
| | | TAT TAC CAG AAT | | | | | | | | | |
| | | CAT AAC CAG AAT | | | | | | | | | |
| | | AGT CAT TAA A | | | | | | | | | |
| papA | sous-unité fimbriale majeure | ACA CCT GAA AAT | 70 | 503- | 69.4 | 40 | X02921 | aucun | 70-papA | CFT073 | |
| (7-1) | des pili P (type F7-1), autre | GTC AAT GAC ACT | | 434 | | | | | (7-1)503 | | |
| | nom: KS71A | GTA CCT TTT TTA | | | | | | | | | |
| | | GCT GCC CCG CCT | | | | | | | | | |
| | | TGA AGC TGT TTC | | | | | | | | | |
| | | AAA TTA GTA A | | | | | | | | | |
| papA | sous-unité fimbriale majeure | TCT GCG GAC CAC | 70 | 536- | 74.9 | 60 | M12861 | aucun | 70-papA | CFT073 | |
| (7-2) | des pili P (type F7-2) | TTG GGA CAC CCG | | 467 | | | | | (7-2)536 | | |
| | | AAA AAG TCA GAG | | | | | | | | | |
| | | ATA CTG TGC CAG | | | | | | | | | |
| | | TCT TCG CCC CAC | | | | | | | | | |
| | | CAC CGC CAG C | | | | | | | | | |
| papA | sous-unité fimbriale majeure | AAA GCT AAC TTC | 70 | 317- | 69.8 | 41.4 | Y08931 | aucun | 70-papA | | |
| (8) | des pili P (type F8), autre | ACC GTC CCT GCT | | 248 | | | | | (8)317 | | |
| | nom: feiA | TTT GCA GTA CCA | | | | | | | | | |
| | | CCT ACA GCA CTT | | | | | | | | | |
| | | GGT TTT TTG AAT | | | | | | | | | |
| | | GCA GTA ATA T | | | | | | | | | |
| papA | sous-unité fimbriale majeure | CTG CAG GCA CAC | 70 | 376- | 74 | 57.1 | M68059 | aucun | 70-papA | | |
| (9) | des pili P (type F9) | CTG CAA AAG TCA | | 307 | | | | | (9)376 | | |
| | | GGG ATA CCG TAC | | | | | | | | | |
| | | CTG TCT TAG CTG | | | | | | | | | |
| | | CAC CGC CTG GTG | | | | | | | | | |
| | | TAG CTG CCT T | | | | | | | | | |
| papA | sous-unité fimbriale majeure | CCC CGC TGG TAT | 70 | 331- | 71.3 | 51.4 | Y08927 | papA(40) | 70-papA | | |
| (10) | des pili P (type F10), autre | CTA ACT CCT CAT | | 262 | | | | | (10)331 | | |
| | nom: fteA | TAT GAC CAG AAA | | | | | | | | | |
| | | CCC TTG GAC CAC | | | | | | | | | |
| | | TAA AAG CCA GCT | | | | | | | | | |
| | | TCA CAG TCC C | | | | | | | | | |
| papA | sous-unité fimbriale majeure | CGT ACC GCC GTT | 70 | 1535- | 70.8 | 48.6 | L07420 | f165(1)A | 70-papA | | |
| (11) | des pili P (type F11) | AGT TGC TAA TTC | | 1466 | | | | | (11)1535 | | |
| | | TTC AGC CTG CCC | | | | | | | | | |
| | | CGT TAC TTG TGG | | | | | | | | | |
| | | CCC AGT AAA AGA | | | | | | | | | |
| | | TAA TTG AAC C | | | | | | | | | |
| papA | sous-unité fimbriale majeure | ATT GTA TTA TCC | 70 | 389- | 69.7 | 42.9 | X62157 | fsiA (papA(16)) | 70-papA | | |
| (12) | des pili P (type F12) | CCA TCG ACA AGA | | 320 | | | | | (12)389 | | |
| | | CTT GAC ACA CCT | | | | | | | | | |
| | | GTC GCT GTT GCT | | | | | | | | | |
| | | CCA TCA AAT TTT | | | | | | | | | |
| | | ACT GCT TTG C | | | | | | | | | |
| papA | sous-unité fimbriale majeure | ATC GGG CCA GTA | 70 | 2082- | 70.8 | 44.3 | X61239 | aucun | 70-papA | J96 | UPEC |
| (13) | des pili P (type F13) | AAA GCC AGC TTA | | 2013 | | | | | (13)2082 | O4:K12 | |
| | | ACA GTC CCT TTT | | | | | | | | (SB18) | |
| | | TTG GCG CCA TTA | | | | | | | | | |
| | | CCA CCT TTA AAG | | | | | | | | | |
| | | GCA GTA ATA T | | | | | | | | | |
| papA | sous-unité fimbriale majeure | TTA TTG TTC CCA | 70 | 311- | 74.1 | 54.3 | Y08928 | aucun | 70-papA | | |
| (14) | des pili P (type F14), autre | CTG GAT ACG CCG | | 242 | | | | | (14)311 | | |
| | nom: ffoA | GAA AAA GTC AGA | | | | | | | | | |
| | | GCC GCC GTT CCT | | | | | | | | | |
| | | GCT TTG GTT GCC | | | | | | | | | |
| | | CCA CCA CCA A | | | | | | | | | |
| papA | sous-unité fimbriale majeure | ATG GTA TTA TCT | 70 | 410- | 69.4 | 42.9 | Y08929 | fsiA (papA(16)) | 70-papA | | |
| (15) | des pili P (type F15), autre | CCG TCC ACA AGA | | 341 | | | | | (15)410 | | |
| | nom: ffiA | GTT GAT GCG TCT | | | | | | | | | |
| | | GTC GGA GTT GCA | | | | | | | | | |
| | | CCA TCA AAT TTT | | | | | | | | | |
| | | ACT GGT TTT C | | | | | | | | | |
| papA | sous-unité fimbriale majeure | ATA GTA TTG TCT | 70 | 407- | 68.9 | 42.9 | Y08930 | ffiA (papA(15)) | 70-papA | | |
| (16) | des pili P (type F16), autre | CCG TCT ACA AGA | | 338 | | | | | (16)407 | | |
| | nom: fsiA | GAG GAT ACA CCT | | | | | | | | | |
| | | GTC GCA GTT GCA | | | | | | | | | |
| | | CCA TCA AAT TTG | | | | | | | | | |
| | | ACA GCT TTA C | | | | | | | | | |
| papA | sous-unité fimbriale majeure | CCC CGC TGG TAT | 70 | 331- | 70.8 | 50 | AF234627 | fteA (papA(10)) | 70-papA | | |
| (40) | des pili P (type F40) | CTA ACT CCT CCT | | 262 | | | | | (40)331 | | |
| | | TAT GAT TAG CAA | | | | | | | | | |
| | | CTA TTG GGC CAG | | | | | | | | | |
| | | TAA AAG CCA GGT | | | | | | | | | |
| | | TCA CAG TCC C | | | | | | | | | |
| papA | sous-unité fimbriale majeure | TGT CAC AAT TAA | 70 | 250- | 66.7 | 31.4 | AF287159 | aucun | 70-papA | | |
| (48) | des pili P (type F48) | CTA ATT CAA TAT | | | | 181 | | | (48)250 | | |
| | | CCA AGT TCA TTG | | | | | | | | | |
| | | GCT TGG ATC GAC | | | | | | | | | |
| | | CAT CAT TTT CAA | | | | | | | | | |
| | | GAA AAC TTT T | | | | | | | | | |
| papC | protéine usher des pili P | CAT AGC CGG CTT | 70 | 3189- | 69.5 | 42.9 | X61239 | prfC | 70-papC3189 | CFT073 | UPEC |
| | | CTG AAA AAC GGG | | 3120 | | | | | | | |
| | | TGA AGT CAA TAT | | | | | | | | | |
| | | TTT TCT TGT CCG | | | | | | | | | |
| | | CTG CGT CAA GTA | | | | | | | | | |
| | | CAT CTG TAT T | | | | | | | | | |
| pixA | ssu majeure des pili Pix des | AAA CTT TGA GCA | 70 | 2230- | 70.3 | 45.7 | AJ307043 | prpA (pap- | 70-pixA2230 | X2194 | UPEC |
| | UPEC, pap-related pili | GAA CCT TCA GTA | | 2161 | | | | related pili) | | | |
| | | CCA AAA GAA ACT | | | | | | | | | |
| | | AGC TTA CCG TCC | | | | | | | | | |
| | | TGA CCG GAA ATC | | | | | | | | | |
| | | ACA ACC GCA G | | | | | | | | | |
| pic | protease impliquee dans la | CAC CCG ATA AAA | 70 | 1570- | 68.6 | 41.4 | AF097644 | aucun | 70-pic1570 | 042 | EAEC, |
| | colonisation intestinale | AGC GGT GTA ACG | | 1501 | | | | | | | UPEC |
| | (mucinase), autre nom: | TTC AGT GTA TTT | | | | | | | | | |
| | picU | ATA AGC ATT GGC | | | | | | | | | |
| | | TTT GGT TCC TTC | | | | | | | | | |
| | | TGA TGT TAC C | | | | | | | | | |
| ralG | ssu majeure des fimbrie de | ATC AGA TTT ACC | 70 | 4750- | 68.7 | 42.9 | U84144 | aucun | 70-ralG4750 | | REPEC |
| | REPEC | AAC CAA GAG AGG | | 4681 | | | | | | | |
| | | CGT ACG CTT ATC | | | | | | | | | |
| | | CAT CGT AAT GGT | | | | | | | | | |
| | | TAG AGA ATC CTT | | | | | | | | | |
| | | CTC AGC ATT C | | | | | | | | | |
| malX | PTS système pour maltose et | TTT ATG GCG ATG | 70 | 2285- | 73.8 | 41.4 | AF081286 | aucun | 70-malX2285 | H1408550 | UPEC |
| | glucose (composant IIABC), | CAT CTG GGA ACG | | 2216 | | | | | | (SB35) | |
| | pathogenicity island | AAC TTT TAT CTT | | | | | | | | | |
| | associated (marqueur PAI) | AAA CAG CAC GAC | | | | | | | | | |
| | | TTA TTG GTC GTT | | | | | | | | | |
| | | GCT GAC CAA A | | | | | | | | | |
| pet | enterotoxine autotransporteur, | CCT TTA TTC TGT | 70 | 498- | 73.7 | 44.3 | AF056581 | aucun | 70-pet498 | 042 | EAEC |
| | serine protéase (plasmid | GCC AGA TCG AGA | | 429 | | | | | | | |
| | encoded toxin) | TAA TCC CGG GCC | | | | | | | | | |
| | | CAT GCT TTA GAT | | | | | | | | | |
| | | ATA TCC ATA TTG | | | | | | | | | |
| | | GCG GCA TAT A | | | | | | | | | |
| rfc | antigene O polymerase (O4) | ATA CTA ACG CAG | 70 | 94- | 71.4 | 38.6 | U39042 | aucun | 70-rfc94 | J96 | MENEC |
| | | ATA CAA CAT ATA | | 25 | | | | | | O4:K12 | |
| | | ATG CCT GTC GCC | | | | | | | | (SB18) | |
| | | TGT GTG TTA AAA | | | | | | | | | |
| | | ACG TAC AGA TCA | | | | | | | | | |
| | | TAA ACA GTG C | | | | | | | | | |
| wzx(O6) | flippase, antigene O6 | TCG CAG CAA CCA | 70 | 600- | 68.1 | 37.1 | AJ426045 | aucun | 70-wzx | CFT073 | |
| | | CAG GTC CTG TGT | | 531 | | | | | (O6)600 | | |
| | | AAG TAA AGC CAA | | | | | | | | | |
| | | AAT CAA TAA TCA | | | | | | | | | |
| | | AAG CCA CTA TTT | | | | | | | | | |
| | | GAT AAA TAG A | | | | | | | | | |
| wzy(O7) | antigene O polymerase (O7) | GTA ATA CAA ATA | 70 | 9850- | 68.5 | 40 | AF125322 | aucun | 70-wzy | | |
| | | ACG CTG AAA TTA | | 9781 | | | | | (O7)9850 | | |
| | | CTC CGC CTC CGC | | | | | | | | | |
| | | GCT CAT TAT TAC | | | | | | | | | |
| | | CAG CAA CAA ATA | | | | | | | | | |
| | | AGC CTG TAT T | | | | | | | | | |
| mtfA | mannosyltransférase A, | CGC AGC GCA TCG | 70 | 8370- | 76.2 | 61.4 | D43637 | aucun | 70-mtfA8370 | P81-603A | MENEC |
| | antigene O9 (autre nom: | CTT CCA GCG GCG | | 8301 | | | | | | O9:K- | |
| | wbdA) | GCA GGC CGA AAC | | | | | | | | (SB5) | |
| | | CTT CAT GCA GCG | | | | | | | | | |
| | | ACG GGA ACA CAA | | | | | | | | | |
| | | ACA GTT TGC A | | | | | | | | | |
| wzy | antigene O polymerase | ATA AAT TAA CCA | 70 | 6430- | 66.1 | 31.4 | AF529080 | aucun | 70-wzy | | |
| (O26) | (O26) | GCG ATA ACC AAT | | 6361 | | | | | (O26)6430 | | |
| | | CTC GGC ATA AAG | | | | | | | | | |
| | | TTC ATT GAC ATT | | | | | | | | | |
| | | AAA TAT ATC AAC | | | | | | | | | |
| | | ATA CGC TTC A | | | | | | | | | |
| wzy | antigene O polymerase | AAA CAT AAT AAG | 70 | 9670- | 65.1 | 28.6 | AF461121 | aucun | 70-wzy | | |
| (O55) | (O55) | ACA TTA GCA TTA | | 9601 | | | | | (O55)9670 | | |
| | | GTG TAA CAC ATA | | | | | | | | | |
| | | ACA AAC TTG GGC | | | | | | | | | |
| | | TAA TTC TAA CCT | | | | | | | | | |
| | | CAT CAT TTA T | | | | | | | | | |
| rfb | O-antigen subunit | AAG CAT GAA GAT | 70 | 157- | 70.9 | 35.7 | X59852 | aucun | 70-rfb | h510a | ETEC |
| (O103) | transferase, biosynthèse de | CTG AAT ACA CAT | | 88 | | | | | (O101)157 | O101 | |
| | l'antigene O101 | ACT CAG TTG ACT | | | | | | | | (SB34) | |
| | | TTA ACC CAG GCA | | | | | | | | | |
| | | ATA ATT TTA AAC | | | | | | | | | |
| | | GTG CAG ACA T | | | | | | | | | |
| wzy | antigene O polymerase | ACG CAT GTA GAA | 70 | 7990- | 65.3 | 28.6 | AY532664 | aucun | 70-wzy | | |
| (O103) | (O103) | TAA AAT AAA TAA | | 7921 | | | | | (O103)7990 | | |
| | | AGC ATC AAG TAT | | | | | | | | | |
| | | ATT TAG CCA ACC | | | | | | | | | |
| | | AAA ATT TAG GAC | | | | | | | | | |
| | | AAC TGG ATA T | | | | | | | | | |
| wzy | antigene O polymerase | CAA GTC CAG TGC | 70 | 6970- | 68.5 | 40 | AF381371 | aucun | 70-wzy | | |
| (O104) | (O104) | CGA ACC CTC CTT | | 6901 | | | | | (O104)6970 | | |
| | | GCA AAT GTG CAA | | | | | | | | | |
| | | ATT GGC TAT TGC | | | | | | | | | |
| | | CAT ATA TTT CAT | | | | | | | | | |
| | | TAT AAT ATG G | | | | | | | | | |
| wbdI | gene de l'operon de | TTT TGC GAA TCC | 70 | 3336- | 71.4 | 35.7 | AF0787368 | aucun | 70-wbdI3336 | H87-5457 | EHEC |
| | l'antigene O111 | TAC CAC CTG GAA | | 3267 | | | | | | O111 | |
| | | CAA AAA AAT AAT | | | | | | | | (SB33) | |
| | | TTT TGG CCG GTC | | | | | | | | | |
| | | GAT TAT TCC TAA | | | | | | | | | |
| | | GAC CAA ATA A | | | | | | | | | |
| wzy | antigene O polymerase | ATC ATA CAT GCT | 70 | 4030- | 65.6 | 31.4 | AF172324 | aucun | 70-wzy | | |
| (O113) | (O113) | AAT ACT GAA TAT | | 3961 | | | | | (O113)4030 | | |
| | | ATA ATA AAT GAC | | | | | | | | | |
| | | AAG TGC CTA TAG | | | | | | | | | |
| | | TTT CGC TGG CAT | | | | | | | | | |
| | | ATT ACT GCA T | | | | | | | | | |
| wzy | antigene O polymerase | TCT ATC CTT TCA | 70 | 9970- | 67.4 | 34.3 | AY208937 | aucun | 70-wzy | | |
| (O121) | (O121) | ACA CTA CCG GCT | | 9901 | | | | | (O121)9970 | | |
| | | GTA TTA ACG CCC | | | | | | | | | |
| | | ATT TGT GTG TTA | | | | | | | | | |
| | | AAA ATA ATA AAT | | | | | | | | | |
| | | GCG ATT TGA A | | | | | | | | | |
| rfbE | perosamine synthetase, | GAT ATA CCT AAC | 70 | 328- | 70.7 | 37.1 | S83460 | aucun | 70-rfbE328 | EDL933 | EHEC |
| | synthèse de l'antigene O157 | GCT AAC AAA GCT | | 259 | | | | | | O157:H7 | |
| | (autre nom: per, wbhD) | AAA TGA AGA GCA | | | | | | | | (SB44) | |
| | | ACC GTT CCA TTA | | | | | | | | | |
| | | CTT ACA GTA GTT | | | | | | | | | |
| | | GCA TAT TGC A | | | | | | | | | |
| wzy | antigene O polymerase | TTA TCC TTT GAC | 70 | 1380- | 65.3 | 31.4 | AF061251 | aucun | 70-wzy | STJ348 | EHEC |
| (O157H7) | (O157:H7) | AGG ATA TTG GTA | | 1311 | | | | | (O157:H7) | O157:H7 | |
| | | ATC AAT ATA TAT | | | | | | | 1380 | (SB22) | |
| | | TGA AGA ATG AGC | | | | | | | | | |
| | | AAC ACC AAT TCA | | | | | | | | | |
| | | GAA CGA TAA C | | | | | | | | | |
| rtx | exoproteine supposée de la | TGA CCG GAT GGG | 70 | 837- | 78.1 | 52.9 | AE005229 | aucun | 70-rtx837 | EDL933 | EHEC |
| | famille RTX (autre nom: | TGA TGG TGG ATG | | 768 | | | | | | O157:H7 | |
| | z0615) | TTG TTC CGG CTG | | | | | | | | (SB44) | |
| | | TGT TAG TGC CAC | | | | | | | | | |
| | | TTA CCG TGA TAT | | | | | | | | | |
| | | TCA CCG TAC C | | | | | | | | | |
| saa | STEC autoagglutinating | GAT GCT CTT CCC | 70 | 1810- | 69.9 | 45.7 | AF325220 | aucun | 70-saa1810 | 98NK2 | STEC |
| | adhesin | CCT GCC TCC GTT | | 1741 | | | | | | | |
| | | TTA CCG CTA CCA | | | | | | | | | |
| | | AGA TAT GAC ATC | | | | | | | | | |
| | | TCC GAG TAA ATT | | | | | | | | | |
| | | GCT TTG ATA T | | | | | | | | | |
| sat | toxine sécrétée, | CAA TAT TTG CTG | 70 | 157- | 73.8 | 41.4 | AF289092 | aucun | 70-sat157 | CFT073 | UPEC |
| | autotransporteur (serine | CAT TTA CTG TAC | | 88 | | | | | | | |
| | protease) | CGG CAA CAG CCA | | | | | | | | | |
| | | GAG ACA ACA TTG | | | | | | | | | |
| | | TTG CTA CAA GTT | | | | | | | | | |
| | | TTC GGT TTG T | | | | | | | | | |
| astA | heat-stable enterotoxin 1 | AGG CTG TTG TCG | 70 | 130- | 77.9 | 50 | L11241 | region adjacaente a | 70-astA130 | H-10407 | commun |
| | des E. coli enteroaggregatifs | ACC ATA TGC ACG | | 61 | | | | stb (STII) (43/70), | | (SB29) | |
| | (EASTI), autre nom: eastI | ATG CAT AAC TGG | | | | | | z6017 (ou ecs1817) | | | |
| | | ATG CGG GCC TTC | | | | | | et z2082 (ou ecs2221): | | | |
| | | GGA TAT ACT GTG | | | | | | transposase (63/70) | | | |
| | | TTG ATG GCA T | | | | | | | | | |
| st | heat stable toxin I ou STa | CTC TAC TGG TTT | 70 | 102- | 68.1 | 31.4 | M29255 | esta2 (variant STa2) | 70-st102 | H-10407 | ETEC |
| | (variants esta3 (STa3), esta4 | AGC ATC CTG AGC | | 33 | | | | | | (SB29) | |
| | (Sta4)) autres noms: st-Ib, | GAA AGG TGA AAA | | | | | | | | | |
| | st-h | AGA CAA TAC AGA | | | | | | | | | |
| | | AAG AAA AAT AAA | | | | | | | | | |
| | | TAA TAT TGA T | | | | | | | | | |
| esta1 | heat stable toxin I ou STa | GAT TCA GTT GAC | 70 | 365- | 68.5 | 31.4 | M58746 | aucun | 70-esta1365 | P97- | ETEC, |
| | (variant ESTa1 ou STa1), | TGA CTA AAA GAG | | 296 | | | | | | 2554B | VTEC |
| | autres noms: st-Ia, st-p | GGG AAA GAT AAT | | | | | | | | O149:K91 | |
| | | ACA GAA ATA AAA | | | | | | | | (SB9) | |
| | | ATT GCC AAC ATT | | | | | | | | | |
| | | AGC TTT TTC A | | | | | | | | | |
| stlI | heat stable toxin II (stII | ATG CAT AGG CAT | 70 | 512- | 69.4 | 31.4 | M35586 | aucun | 70-stlI1512 | P97- | ETEC, |
| | (STII), autres noms: stb) | TTG TAG CAA TAG | | 443 | | | | | | 2554B | VTEC |
| | | AAA AAA CGA ACA | | | | | | | | O149:K91 | |
| | | TAG ATG CAA GAA | | | | | | | | (SB9) | |
| | | GAA ATG CGA TAT | | | | | | | | | |
| | | TCT TTT TCA T | | | | | | | | | |
| stx1A | shiga-like toxin 1 - ssu A | CAT CCC CGT ACG | 70 | 742- | 70.9 | 51.4 | AF461168 | nombreux variants | 70-stx1A742 | EDL933 | EHEC |
| | (autres noms: slt-IA, stx1 | ACT GAT CCC TGC | | 673 | | | | de stx1A: c, d, | | O157:H7 | |
| | ou stxA) | AAC ACG CTG TAA | | | | | | v51, v52 | | (SB44) | |
| | | CGT GGT ATA GCT | | | | | | | | | |
| | | ACT GTC ACC AGA | | | | | | | | | |
| | | CAA TGT AAC C | | | | | | | | | |
| stx1B | shiga-like toxine I ssuB, | TCA TCC CCG TAA | 70 | 1454- | 67.6 | 38.6 | AF461168 | slt-IB, stx1vB, | 70-stx1 | EDL933 | EHEC |
| | autres noms: stx1B, stx1, | TTT GCG CAC TGA | | 1385 | | | | variant d, v51, | B1454 | O157:H7 | |
| | stxB | GAA GAA GAG ACT | | | | | | V52 | | (SB44) | |
| | | GAA GAT TCC ATC | | | | | | | | | |
| | | TGT TGG TAA ATA | | | | | | | | | |
| | | ATT CTT TAT C | | | | | | | | | |
| stx2A | shiga-like toxin II - ssu A, | GTA TTA CCA CTG | 70 | 1087- | 69.1 | 44.3 | X65949 | tous les variants | 70-stx2 | EDL933 | EHEC |
| | autre nom: slt-IIA, slt-IIvA, | AAC TCC ATT AAC | | 1018 | | | | sauf f (stx2tA) | A1087 | O157:H7 | |
| | slt-IIeA, vtx2a, vta | GCC AGA TAT GAT | | | | | | | | (SB44) | |
| | | GAA ACC AGT GAG | | | | | | | | | |
| | | TGA CGA CTG ATT | | | | | | | | | |
| | | TGC ATT CCG G | | | | | | | | | |
| stx2B-1 | shiga-like toxine II - ssuB, | AAA TCC GGA GCC | 70 | 7335- | 75.1 | 45.7 | AE005296 | slt-IIeB, slt-IIvB, | 70-stx2 | EDL933 | EHEC |
| | autres noms: vtB, stxII, | TGA TTC ACA GGT | | 7266 | | | | VT2vaB, nombreux | B(1)7335 | O157:H7 | |
| | stx2, slt-IIB | ACT GGA TTT GAT | | | | | | variants: c, d, e, g, | | (SB44) | |
| | | TGT GAC AGT CAT | | | | | | vhd, vhc, NV206, slt- | | | |
| | | TCC TGT CAA CTG | | | | | | IIvtB | | | |
| | | AGC ACT TTG C | | | | | | | | | |
| stx2B-2 | shiga-like toxine II ssuB - | AAA TCC TGA ACC | 70 | 1790- | 74.2 | 4.29 | X65949 | nombreux variants: | 70-stx2 | OX3:H21 | EHEC |
| | (variant) | TGA CGC ACA GGT | | 1721 | | | | d, g, NV206, c, vhd, | B(2)1790 | | |
| | | ATT TGA TTT GAT | | | | | | vhc, et VT2b, VT2vaB, | | | |
| | | TGT TAC CGT CAT | | | | | | slt-IIvtB | | | |
| | | TCC TGT TAA CTG | | | | | | | | | |
| | | TGC GCT TTG C | | | | | | | | | |
| stlV-IIvB | shiga-like toxine II - ssuB | AAA GCC TGA GCC | 70 | 1418- | 74.6 | 44.3 | M36727 | nombreux variants: | 70-stlV- | h510a | EHEC |
| | (variant) | TCA ACT GCA GGT | | 1349 | | | | e, f, t, vhc, vhd, | IIvB1418 | O101 | |
| | | ATT AGA TAT GAT | | | | | | c, d, slt-IIvaB, | | (SB34) | |
| | | TGT TAC AGT CAT | | | | | | slt-IIeB, VT2vaB | | | |
| | | CCC TGT CAG CTG | | | | | | | | | |
| | | AGC ACT TTG T | | | | | | | | | |
| stx2tA | shiga toxin II - ssuA (variant | CAT CTG CAT AAG | 70 | 137- | 70.3 | 34.3 | AJ010730 | slt-IIvA | 70-stx2 | T4/97 | EHEC |
| | t), autre nom: stx2fA | ATG CTG AAG ACA | | 68 | | | | | tA137 | O128:H2 | |
| | | AGC AAA CAC AAA | | | | | | | | | |
| | | AAA ACA ACA CCA | | | | | | | | | |
| | | GCT TTA ATA ATA | | | | | | | | | |
| | | TAT GTC GCA T | | | | | | | | | |
| stx2tB | shiga toxine II - ssuB | TTC CTA CAG CAC | 70 | 1115- | 73.8 | 41.4 | AJ010730 | slt-IIvaB, variant f | 70-stx2 | T4/97 | EHEC |
| | (variant t), autre nom: | AAT CCG CCG CCA | | 1046 | | | | et t, faiblement avec | tB1115 | O128:H2 | |
| | stx2fB | TGG AAT TAG CAG | | | | | | autres variants | | | |
| | | AAA AGA GAC CGA | | | | | | (e, c, . . . ) | | | |
| | | ATA AAA CTG CAA | | | | | | | | | |
| | | TAA TCA TCT T | | | | | | | | | |
| set | enterotoxine supposee | TTT TGA AGG GCC | 70 | 217630- | 66.4 | 32.9 | AP002563 | aucun | 70-set | EDL933 | commun |
| | (homologue a ShET: | TGA TAT AAA CCA | | 217561 | | | | | 217630 | O157:H7 | |
| | enterotoxine de S. flexneri) | GGT ATG GTT CCA | | | | | | | | (SB44) | |
| | | TCC AAA GTT CTT | | | | | | | | | |
| | | GCA GAT AAT ATA | | | | | | | | | |
| | | TGT ATT AAT T | | | | | | | | | |
| senB | enterotoxine des EIEC | CAC AAA GGC ACG | 70 | 1030- | 73.1 | 54.3 | Z54195 | aucun | 70-senB1030 | | EIEC, |
| | | GTC AGA AGC GGA | | 961 | | | | | | | MENEC |
| | | GTC CAC CGC CAG | | | | | | | | | |
| | | ATT CTG CAC ACT | | | | | | | | | |
| | | TGT GAT TTG TGG | | | | | | | | | |
| | | TCT CGG ATC T | | | | | | | | | |
| shf | protéine cryptique sécrétée, | TTC CGG AAT GTC | 70 | 670- | 70.4 | 47.1 | AF134403 | aucun | 70-shf670 | | EAEC, |
| | plasmide pAA2 des EAEC | TCG GGA GAA AGT | | 601 | | | | | | | DAEC |
| | (impliquée dans l'adhesion | GTA ACC AGT CCT | | | | | | | | | |
| | des EAEC?) | GGG CAA TGG CTG | | | | | | | | | |
| | | ACA TGA TGA TAC | | | | | | | | | |
| | | ATT AAT ACC G | | | | | | | | | |
| tia | proteine d'invasion des | AAT ATC ACT TAT | 70 | 534- | 68.6 | 41.4 | U20318 | aucun | 70-tia534 | H10407 | ETEC |
| | ETEC | CTC GCC AGA TTC | | 465 | | | | | | | |
| | | ATT CCA GGA GGT | | | | | | | | | |
| | | ATC AAT ATA TGT | | | | | | | | | |
| | | CGC CTT ATG ATG | | | | | | | | | |
| | | TAC CCG TGC A | | | | | | | | | |
| tibA | proteine d'adhesion et | GCG CTC CGC TGG | 70 | 550- | 73.5 | 55.7 | AF109215 | aucun | 70-tibA550 | H10407 | ETEC |
| | d'invation des ETEC, | TAA CAG ATG CGC | | 481 | | | | | | | |
| | (glycoproteine) | TTG TGG CAC TGC | | | | | | | | | |
| | | CAC CAC TGA TTA | | | | | | | | | |
| | | CAT ACT GAT CTC | | | | | | | | | |
| | | CTC CGC TGT T | | | | | | | | | |
| tir-1 | translocated intimin | ACC ATG CAA AGA | 70 | 345- | 77.7 | 50 | AF045568 | espE | 70-tir | RDEC-1B | EHEC, |
| | recepetor group I, autre | TAC TTC GGA CGC | | 276 | | | | | (1)345 | O15 | EPEC |
| | nom: espE | AGC AAA GCG CAG | | | | | | | | (SB40) | |
| | | TGG ATT TGT AGG | | | | | | | | | |
| | | AAG TCC GGG AAT | | | | | | | | | |
| | | ATC ACT GGC A | | | | | | | | | |
| tir-2 | translocated intimin | ATC ATT CAG TGT | 70 | 1557- | 78.1 | 51.4 | AF070067 | aucun | 70-tir | EDL933 | EHEC, |
| | recepetor groupe II, autre | TAT CTC AGA CGC | | 1488 | | | | | (2)1557 | O157:H7 | EPEC |
| | nom: espE | CGC CAG GCG CAT | | | | | | | | (SB44) | |
| | | CGG ATT TAC AGG | | | | | | | | | |
| | | AAG TCC AGG AAC | | | | | | | | | |
| | | ATC ACT GGC A | | | | | | | | | |
| tir-3 | translocated intimin | TCC TAA TGC TCC | 70 | 154- | 78.4 | 52.9 | AB036053 | aucun | 70-tir | E2348/69 | EHEC, |
| | recepetor groupe III, | TGT AGA GCT AAT | | 85 | | | | | (3)154 | O126:H6 | EPEC |
| | autre nom: espE | TAG ATG ACC AGT | | | | | | | | (SB28) | |
| | | TCC TCC CCG TGC | | | | | | | | | |
| | | CGC GCC GTC TGT | | | | | | | | | |
| | | TTG TGA AGG T | | | | | | | | | |
| trirA | proteine de resistance au | CAG CAA TCT ACG | 70 | 5993- | 71.5 | 50 | AF126104 | terF | 70-trir | EDL933 | EHEC |
| | tellurium, autre nom: terF | ATC AGG CTG AAT | | 5924 | | | | | A5993 | O157:H7 | |
| | | CTT CAG TAC CCT | | | | | | | | (SB44) | |
| | | GCC AAA TCC GGC | | | | | | | | | |
| | | TTT AAA GGC GAA | | | | | | | | | |
| | | CCC GAT ACC T | | | | | | | | | |
| traT | proteine de resistance au | CTG GCG GGT TCA | 70 | 548- | 76.8 | 51.4 | J01769 | aucun | 70-traT548 | B79-3292 | UPEC, |
| | complement | AGC CAG ATG GTC | | 479 | | | | | | (SB24) | commun |
| | | TCA CTC ATC TGA | | | | | | | | | |
| | | GTC TTC ACC TCA | | | | | | | | | |
| | | AGG TTA CGC TTC | | | | | | | | | |
| | | TTG ATT GCT G | | | | | | | | | |
| tsh | temperature sensitive | GTC TGA CAG ACT | 70 | 4223- | 77.9 | 51.4 | AF218073 | hbp (hemoglobin | 70-tsh4223 | Av 89- | APEC, |
| | hemaggluitinin (hemoglobin | TAT GAA CAC ATT | | 4154 | | | | protease): tsh | | 7098 | commun |
| | protease) | TCC TGG CAA ACT | | | | | | humain | | (143) | |
| | | CAG ATA CGG CAA | | | | | | | | O78:K80 | |
| | | TAA AGC CCC GGG | | | | | | | | (SB10) | |
| | | CCA CAG CGC T | | | | | | | | | |
| uidA | béta-D-glucuronidase, | CCA GAC TGA ATG | 70 | 70- | 77.2 | 50 | S69414 | aucun | 70-uidA70 | EDL933 | commun |
| | autres noms: gusA ou gurA | CCC ACA GGC CGT | | 1 | | | | | | O157:H7 | |
| | | CGA GTT TTT TGA | | | | | | | | (SB44) | |
| | | TTT CAC GGG TTG | | | | | | | | | |
| | | GGG TTT CTA CAG | | | | | | | | | |
| | | GAC GTA ACA T | | | | | | | | | |
| usp | uropathogenic specific | TGA GTA CGC CAC | 70 | 70- | 77.2 | 50 | AB027193 | aucun | 70-usp70 | h1408550 | UPEC, |
| | protein | TGA GCG ACC ATT | | 1 | | | | | | (SB35) | commun |
| | | TTC CCC ATA TTT | | | | | | | | | |
| | | GAG TCG CCA ACA | | | | | | | | | |
| | | CAC TAC TCG GGA | | | | | | | | | |
| | | ACA GTA GCA T | | | | | | | | | |
| virK | protéine impliquée dans | TGG TAA TTT GTA | 70 | 3250- | 69.5 | 42.9 | AF134403 | aucun | 70-virK3250 | | EAEC, |
| | l'invasion (facteur de | CCA GTC ACC ACA | | 3181 | | | | | | | DAEC |
| | virulence lìé a virG chex S. | GGT TTT TCC TGG | | | | | | | | | |
| | flexneri), plasmide pAA2 des | TAC AGA ATC CCA | | | | | | | | | |
| | EAEC | GAA ATC ACT ATA | | | | | | | | | |
| | | GAC CGC AAC A | | | | | | | | | |
| yja | fonction inconnue | GAT TAC GAC GAA | 70 | 210663- | 69.5 | 34.3 | AE016770 | aucun | 70-yja | MG1655 | |
| | | TTT GGA TAT ACA | | 210594 | | | | | A210663 | | |
| | | GAA CTG ACA TGA | | | | | | | | | |
| | | GAT TCC CTT CAT | | | | | | | | | |
| | | CAT GCA AAT AAT | | | | | | | | | |
| | | TGA TAT GCA A | | | | | | | | | |
| mviM | facteur de virulence supposé | TAA CGT ACT GAC | 70 | 1626- | 73.4 | 52.9 | AE005317 | aucun | 70-mviM1626 | EDL933 | commun |
| | | CAC GTC AAA GTG | | 1557 | | | | | | O157:H7 | |
| | | ACT GGC GGT GCT | | | | | | | | (SB44) | |
| | | GGA ATG TAC AAA | | | | | | | | | |
| | | AAC CGC ATC GCA | | | | | | | | | |
| | | ACT GGC GGC A | | | | | | | | | |
| mviN | facteur de virulence supposé | GTC ACA ACC GCC | 70 | 2706- | 73.7 | 57.1 | AE005317 | aucun | 70-mviN2706 | EDL933 | commun |
| | | AGC GCA AGT GTC | | 2637 | | | | | | O157:H7 | |
| | | AGC AGG CCA GAA | | | | | | | | (SB44) | |
| | | ACA TAA GAG ACA | | | | | | | | | |
| | | AAG ACC CGC GTG | | | | | | | | | |
| | | GCG TCT TCA C | | | | | | | | | |
| b1432 | facteur de virulence | TTT AAC CCA GCC | 70 | 10390- | 71.7 | 48.6 | AE016767 | aucun | 70-b(1432) | CFT073 | UPEC |
| | supposé, autre nom: ydcM | CAG TCC TGA CGG | | 10321 | | | | | 10390 | | |
| | | GAG TTT CAC ACG | | | | | | | | | |
| | | GCC ATA ATC CAG | | | | | | | | | |
| | | CCC ACA ATA TTT | | | | | | | | | |
| | | GCT GAA ATT G | | | | | | | | | |
| b1121 | homologue de facteur de | TAT CAG GCT TTA | 70 | 122153- | 69 | 42.9 | AE016759 | aucun | 70-b(1121) | MG1655 | |
| | virulence, autre nom: ycfZ | TGT TTG TAT ATC | | 122084 | | | | | 122153 | | |
| | | GAT AAT AGC TTT | | | | | | | | | |
| | | GCG ATT ACC AGA | | | | | | | | | |
| | | ATA TCG CCA CTC | | | | | | | | | |
| | | TGG GCA GGG C | | | | | | | | | |
| ECs1282 | proteine filamenteuse, | GCA TCC GCC CCG | 70 | 214810- | 75.7 | 64.3 | AP002554 | aucun | 70-ECs | EDL933 | EHEC |
| | hemagglutinin supposée | CTG GTG ACC AGA | | 214741 | | | | | (1282) | O157:H7 | |
| | (similar to hemagglutinin/ | GCA CGC GTG TTG | | | | | | | 214810 | (SB44) | |
| | hemolysin-related proteins) | TCG AAC GTG TTC | | | | | | | | | |
| | | TGC GCC TGC AGA | | | | | | | | | |
| | | GTC AGA GGA C | | | | | | | | | |
| tnaA | tryptophanase | AAA GAC TGG ACC | 70 | 1274- | 84 | 52.9 | K00032 | aucun | 70-tnaA-rb | MG1655 | commun |
| | | ATC GAG CAG ATC | | 1343 | | | | | | | |
| | | ACC CGC GAA ACC | | | | | | | | | |
| | | TAC AAA TAT GCC | | | | | | | | | |
| | | GAT ATG CTG GCG | | | | | | | | | |
| | | ATG TCC GCC A | | | | | | | | | |
| lacY-Ec | lactose permease | CTG GAA CTG TTC | 70 | 745- | | | | ECLAY | 70-lacY-Ec | MG1655 | commun |
| | | AGA CAG CCA AAA | | 814 | | | | | | | |
| | | CTG TGG TTT TTG | | | | | | | | | |
| | | TCA CTG TAT GTT | | | | | | | | | |
| | | ATT GGC GTT TCC | | | | | | | | | |
| | | TGC ACC TAC G | | | | | | | | | |
| lacY-Cf | Citrobacter freundii lactose | TTT ATT TAC AAT | 70 | 346- | 82.9 | 48.6 | CFU13675 | aucun | 70-lacY-Cf | | |
| | permease | GCC GGC GCT CCG | | 415 | | | | | | | |
| | | GCG ATT GAA GCC | | | | | | | | | |
| | | TAT ATT GAA AAA | | | | | | | | | |
| | | GCC AGC CGC CGA | | | | | | | | | |
| | | AGC AAC TTT G | | | | | | | | | |
| lacZ | beta-galactosidase | ATA TGG GGA TTG | 70 | 2969- | 88 | 62.9 | ECLACZ | aucun | 70-lacZ-Ec | MG1655 | commun |
| | | GTG GCG ACG ACT | | 3038 | | | | | | | |
| | | CCT GGA GCC CGT | | | | | | | | | |
| | | CAG TAT CGG CGG | | | | | | | | | |
| | | AAT TCC AGC TGA | | | | | | | | | |
| | | GCG CCG GTC G | | | | | | | | | |
| gad | glutamate decarbosylase | ACC GTT CGT CGC | 70 | 3664782- | 87.6 | 61.4 | U00096 | 55 matches sur 60 | 70-gad-EcSf | MG1655 | commun |
| | | CCC GGA TAT CGT | | 3664851 | | | | avec Edwardsiella | | | |
| | | CTG GGA CTT CCG | | | | | | tarda | | | |
| | | CCT GCC GCG TGT | | | | | | | | | |
| | | GAA ATC GAT CAG | | | | | | | | | |
| | | TGC TTC AGG C | | | | | | | | | |
| ureD | putative urease accessory | ATG CTG GAT CTC | 70 | 253323- | 80 | 57.1 | AP002554 | aucun | 70-ureD- | EDL933 | O157:H7 |
| | protein d | CGT TTT CAG CGT | | 253392 | | | | | EcO157 | O157:H7 | |
| | | CTG CAC GGG AAA | | | | | | | | (SB44) | |
| | | ACC ACG CTC ACC | | | | | | | | | |
| | | ACC CGT CAT CAT | | | | | | | | | |
| | | GTC GGT CTG C | | | | | | | | | |
| sf0315 | unknown | GAGCACGGCAGGA | 70 | 7757- | 79.9 | 44.3 | AE015065.1 | aucun | 70-Sf0315 | | |
| | | ATAATCAAATAGAT | | 7826 | | | | | | | |
| | | GGAATGCGGGGGT | | | | | | | | | |
| | | TCTTAGCAATTTTC | | | | | | | | | |
| | | GTGCTTATTCATCA | | | | | | | | | |
| | | CG | | | | | | | | | |
| sf3004 | unknown | ATGGACGCAACAG | 70 | 7948- | 83.5 | 51.4 | AE015313.1 | aucun | 70-Sf3004 | | |
| | | GCAACACGACAGTC | | 8017 | | | | | | | |
| | | ACCTGCCTGAGTCA | | | | | | | | | |
| | | CAAAATGAAGTACA | | | | | | | | | |
| | | AAGAAGTCGCCTGCG | | | | | | | | | |
| nleA | non-LEE encoded effector A | GAA CGG AAC TGG | 70 | 712- | 67.4 | 35.7 | AY430401 | espI | 70-nleA712 | | EHEC |
| | (type III secreted effector), | GTA TCT CTA ATG | | 643 | | | | | | | (O157:H7) |
| | identique a espI | CCA TTT GAG TAA | | | | | | | | | |
| | | CAT TGA ATA AAC | | | | | | | | | |
| | | CAA ACG TAT CCA | | | | | | | | | |
| | | ATG CTT TTT T | | | | | | | | | |
| cif | cell cycle inhibiting factor | GTG GTC ATC ACT | 70 | 585- | 68.3 | 40 | AF497476 | cif tronqués | 70-cif585 | | EPEC, |
| | | ATT TAG CAA TAC | | 516 | | | | | | | EHEC |
| | | ATT AGC TTT GAG | | | | | | | | | |
| | | GTT CTG TGA GCA | | | | | | | | | |
| | | CAG GGA AGC AAA | | | | | | | | | |
| | | ATC TCT TAC A | | | | | | | | | |
| eae | intimine, variant gamma 2 | CAA ATA AAT ATA | 70 | 16651- | 65.1 | 31.4 | AF071034 | eae gamma like, | 70-eae | EDL933 | EHEC |
| (gamma2) | | GCC ATT ATA GTT | | 16582 | | | | mu, sigma | (gamma2) | | |
| | | CTA TGA ACT CAA | | | | | | | 16651 | | |
| | | TAA CTG CTT GGA | | | | | | | | | |
| | | TTA AAC AGA CAT | | | | | | | | | |
| | | CTA GTG AGC A | | | | | | | | | |
| astA(2) | heat-stable enterotoxin 1 | TGC ACG ATG CAT | 70 | 183- | 73.3 | 54.3 | S81691 | aucun | 70-astA | H10707-P | ETEC |
| | (autre nom: eastI), 8aa en | AAC TGG ATG CGG | | 114 | | | | | (2)183 | | |
| | moins | GCC TTC GGA TAT | | | | | | | | | |
| | | ACT GTG TTG ATG | | | | | | | | | |
| | | GCA TCC GGG AAG | | | | | | | | | |
| | | CCT TTC AGG C | | | | | | | | | |
| bfpA(2) | sous-unite fimbriale majeure | TCC CCC CCA AAT | 70 | 3021- | 68.8 | 37.1 | U27184 | tous les variants | 70-bfpA | | |
| | (BFP: bundle-forming pili), | GGG TTG GTT ATT | | 2952 | | | | alpha et beta | (2)3021 | | |
| | oligo 2 | TTT TTG TTT GTT | | | | | | | | | |
| | | GTA TCT TTG TAA | | | | | | | | | |
| | | TTA TCC GGA ATT | | | | | | | | | |
| | | GCA GAT GTG T | | | | | | | | | |
| bfpA(3) | sous-unite fimbriale majeure | ATA TTA ACA CCG | 70 | 3156- | 69.3 | 41.4 | U27184 | tous les variants | 70-bfpA | | |
| | (BFP: bundle-forming pili), | TAG CCT TTC GCT | | 3087 | | | | alpha et beta | (3)3156 | | |
| | oligo 3 | GAA GTA CCT AAG | | | | | | | | | |
| | | TTC AAG GTT GCA | | | | | | | | | |
| | | AGA CTA ACA CAT | | | | | | | | | |
| | | GCC GCT TTA T | | | | | | | | | |
| lpfA | lpfA des ehec | AAA GTT TAA CCT | 70 | 660- | 70.4 | 44.3 | AY057066 | aucun | 70-lpfA | | |
| (EHEC) | | GCG AAT TAT CGG | | 591 | | | | | (EHEC)660 | | |
| | | ACT GGT TAA AAA | | | | | | | | | |
| | | TAC GAA TAC CAA | | | | | | | | | |
| | | CGC CGG TTG CCG | | | | | | | | | |
| | | CAA TCG CTT G | | | | | | | | | |
| iutA(2) | récepteur de la cloacine | CAC TCC GGT ACT | 70 | 1977- | 72.7 | 55.7 | X05874 | aucun | 70-iut | | |
| | DF13 (aerobactine), ancien | CCA GTC AGT ATC | | 1908 | | | | | (A2)1977 | | |
| | nom DF13 | AGG AAT CAG GTA | | | | | | | | | |
| | | GTC CAC CGC ACC | | | | | | | | | |
| | | TTC CAC GCC GTA | | | | | | | | | |
| | | AAT ACG GCG T | | | | | | | | | |
| iut | récepteur de l'aérobactine, | GCG CCG TAT TTA | 70 | 134328- | 73.7 | 60 | AE016766 | aucun | 70-iut | CFT073 | |
| (upec) | souche CFT073 | CGG CGT GGA AGG | | 134259 | | | | | (upec) | | |
| | | TGC GGT GGA CTA | | | | | | | 134328 | | |
| | | CCT GAT CCC GGA | | | | | | | | | |
| | | TAC TGA CTG GAG | | | | | | | | | |
| | | TAC CGG TGT G | | | | | | | | | |
| int1(2) | integron de classe 1, region | GGC TGT AAT TAT | 70 | 2368- | 72.2 | 52.9 | AY152821 | aucun | 70-int1 | | |
| | conservée, qacEdelta1 | GAC GAC GCC GAG | | 2299 | | | | | (2)2368 | | |
| | | TCC CGA CCA GAC | | | | | | | | | |
| | | TGC ATA AGC AAC | | | | | | | | | |
| | | ACC GAC AGG GAT | | | | | | | | | |
| | | GGA TTT CAG A | | | | | | | | | |
| int1(3) | intégron de classe 1, | CGT TCG GTC AAG | 70 | 284- | 71.5 | 51.4 | AY781413 | aucun | 70-int1(3) | | |
| | intégrase | GTT CTG GAC CAG | | 215 | | | | | | | |
| | | TTG CGT GAG CGC | | | | | | | | | |
| | | ATA CGC TAC TTG | | | | | | | | | |
| | | CAT TAC AGT TTA | | | | | | | | | |
| | | CGA ACC GAA C | | | | | | | | | |

Antibiotic resistance

| | | | | | | | | | |
|---|---|---|---|---|---|---|---|---|---|
| tem | β-lactamines (ampicilline) | AAA GTT CTG CTA | 70 | 8674- | 80.4 | 57.1 | tem(X) | AF307748 | 70-tem8674 |
| | | TGT GGC GCG GTA | | 8605 | | | | | |
| | | TTA TCC CGT GTT | | | | | | | |
| | | GAC GCC GGG CAA | | | | | | | |
| | | GAG CAA CTC GGT | | | | | | | |
| | | CGC CGC ATA C | | | | | | | |
| shv | β-lactamines (ampicilline) | CTC AAG CGG CTG | 70 | 86- | 83.7 | 64.3 | shv(X) | AF148850 | 70-shv86 |
| | | CGG GCT GGC GTG | | 17 | | | | | |
| | | TAC CGC CAG CGG | | | | | | | |
| | | CAG GGT GGC TAA | | | | | | | |
| | | CAG GGA GAT AAT | | | | | | | |
| | | ACA CAG GCG A | | | | | | | |
| oxa-1 | β-lactamines (ampicilline) | AAA CAA CCT TCA | 70 | 256- | 74.3 | 44.3 | oxa-1 | AJ238349 | 70-oxa |
| | | GTT CCT TCA AAT | | 187 | | | | | (1)256 |
| | | AAT GGA GAT GCG | | | | | | | |
| | | ACA GTA GAG ATA | | | | | | | |
| | | TCT GTT GAT GCA | | | | | | | |
| | | CTG GCG CTG C | | | | | | | |
| oxa-7 | β-lactamines (ampicilline) | GTA GCG CAG GCT | 70 | 295- | 75.2 | 45.7 | oxa-13, | X75562 | 70-oxa |
| | | AAT TTA CTG CAT | | 226 | | | oxa-19, | | (7)295 |
| | | CTT TTA CAA AGC | | | | | oxa-14, | | |
| | | ACG AAA ACA CCA | | | | | pse-2, | | |
| | | TTG ACG GCT TCG | | | | | oxa-10, | | |
| | | GCA GAG AAC T | | | | | oxa-17, | | |
| | | | | | | | oxa-16, | | |
| | | | | | | | oxa-7 | | |
| pse-4 | β-lactamines (ampicilline) | CGC TGA TTG CCA | 70 | 348- | 72.3 | 41.4 | pse-4, | J05162 | 70-pse |
| | | TTG TAA TCC CAA | | 279 | | | pse-5, | | (4)348 |
| | | TAT TCT CCA TTT | | | | | carb-6, | | |
| | | TGA GTA TCA AGA | | | | | pse-1 | | |
| | | ACG GAA ACA CCT | | | | | | | |
| | | ATA CGA GCA G | | | | | | | |
| ctx | β-lactamines (ampicilline) | ATA CAG CGG CAC | 70 | 143- | 80.3 | 55.7 | ctx-m-1, | X92506 | 70-ctx143 |
| | | ACT TCC TAA CAA | | 74 | | | ctx-m-3, | | |
| | | CAG CGT GAC GGT | | | | | ctx-m-28, | | |
| | | TGC CGT CGC CAT | | | | | ctx-m-11, | | |
| | | CAG CGT GAA CTG | | | | | ctx-m-27, | | |
| | | ACG CAG TGA | | | | | ctx-m-22, | | |
| | | | | | | | ctx-m-27, | | |
| | | | | | | | ctx-m-15 | | |
| ant(3″)-Ia | streptomycine, | ATG ATG TCG TCG | 70 | 290- | 79.2 | 55.7 | aadA1, | X12870 | 70-aadA |
| (aadA1) | spectinomycine | TGC ACA ACA ATG | | 221 | | | aadA2 | | (1)290 |
| | | GTG ACT TCT ACA | | | | | | | |
| | | GCG CGG AGA ATC | | | | | | | |
| | | TCG CTC TCT CCA | | | | | | | |
| | | GGG GAA GCC G | | | | | | | |
| ant(2″)-Ia | kanamycine, neomycine, | CCC GAG TGA GGT | 70 | 1778- | 79.1 | 55.7 | aadB | M86913 | 70-aadB1778 |
| (aadB) | gentamicine | GCA TGC GAG CCT | | 1709 | | | | | |
| | | GTA GGA CTC TAT | | | | | | | |
| | | GTG CTT TGT AGG | | | | | | | |
| | | CCA GTC CAC TGG | | | | | | | |
| | | TGG TAC TTC A | | | | | | | |
| aac(3)IIa | gentamicine | CAC CGG TTT GGA | 70 | 200- | 77.7 | 52.3 | aacC2 | S68058 | 70-aacC |
| (aacC2) | | CTC CGA GTT TTC | | 131 | | | | | (2)200 |
| | | GAA TTG CCT CCG | | | | | | | |
| | | TTA TTG CCT TCC | | | | | | | |
| | | GCG TAT GCA TCG | | | | | | | |
| | | CGA TAT CTC C | | | | | | | |
| aac(3)-IV | gentamicine | TCG ATC AGT CCA | 70 | 380- | 82.7 | 62.9 | aac(3)- | X01385 | 70-aac3 |
| | | AGT GGC CCA TCT | | 311 | | | IV | | (IV)380 |
| | | TCG AGG GGC CGG | | | | | | | |
| | | ACG CTA CGG AAG | | | | | | | |
| | | GAG CTG TGG ACC | | | | | | | |
| | | AGC AGC ACA C | | | | | | | |
| aph(3′)-Ia | kanamycine, neomycine | GGC GCA TCG GGC | 70 | 1310- | 79.1 | 54.3 | aphA1, | V00359 | 70-aphA |
| (aphA1) | | TTC CCA TAC AAT | | 1241 | | | aphA7, | | (1)1310 |
| | | CGA TAG ATT GTC | | | | | strA, | | |
| | | GCA CCT GAT TGC | | | | | Tn903 | | |
| | | CCG ACA TTA TCG | | | | | | | |
| | | CGA GCC CAT T | | | | | | | |
| aph(3′)-IIa | kanamycine, neomycine | AGT CAT AGC CGA | 70 | 220- | 78.9 | 52.9 | Tn5, | V00618 | 70-aphA |
| (aphA2) | | ATA GCC TCT CCA | | 151 | | | aphA2, | | (2)220 |
| | | CCC AAG CGG CCG | | | | | aph(3′) | | |
| | | GAG AAC CTG CGT | | | | | | | |
| | | GCA ATC CAT CTT | | | | | | | |
| | | GTT CAA TCA T | | | | | | | |
| tet(A) | tetracycline | GAT GCC GAC AGC | 70 | 1390- | 79.5 | 57.1 | tetA | X00006 | 70-tetA1390 |
| | | GTC GAG CGC GAC | | 1321 | | | | | |
| | | AGT GCT CAG AAT | | | | | | | |
| | | TAC GAT CAG GGG | | | | | | | |
| | | TAT GTT GGG TTT | | | | | | | |
| | | CAC GTC TGG C | | | | | | | |
| tet(B) | tetracycline | CAA AGT GGT TAG | 70 | 190- | 71.8 | 40 | tetB, | V00611 | 70-tetB190 |
| | | CGA TAT CTT CCG | | 121 | | | Tn10 | | |
| | | AAG CAA TAA ATT | | | | | | | |
| | | CAC GTA ATA ACG | | | | | | | |
| | | TTG GCA AGA CTG | | | | | | | |
| | | GCA TGA TAA G | | | | | | | |
| tet(C) | tetracycline | GAC TGG CGA TGC | 70 | 130- | 80.8 | 58.6 | pBR322, | J01749 | 70-tetC130 |
| | | TGT CGG AAT GGA | | 61 | | | RP1, | | |
| | | CGA TAT CCC GCA | | | | | tetC | | |
| | | AGA GGC CCG GCA | | | | | . . . | | |
| | | GTA CCG GCA TAA | | | | | | | |
| | | CCA AGC CTA T | | | | | | | |
| tet(D) | tetracycline | CAA ACG CGG CAC | 70 | 1770- | 83.5 | 64.3 | tetA | X65876 | 70-tetD1770 |
| | | CCG CCA GGG ATA | | 1701 | | | | | |
| | | ACA GCA GCA CCG | | | | | | | |
| | | GTC TGC GCC CCA | | | | | | | |
| | | GCT TAT CTG ACC | | | | | | | |
| | | ATC TGC CCA G | | | | | | | |
| tet(E) | tetracycline | GTT GAG GCT GCA | 70 | 370- | 78 | 51.4 | tetE | L06940 | 70-tetE370 |
| | | ACA GCT CCA GTC | | 301 | | | | | |
| | | GCA CCG GTA ATA | | | | | | | |
| | | CCA GCA ATT AAG | | | | | | | |
| | | CGT CCC AAA TAC | | | | | | | |
| | | AAC ACC CAC A | | | | | | | |
| tet(Y) | tetracycline | TTA ATA AAG CCG | 70 | 1770- | 76.5 | 47.1 | tetY | AF070999 | 70-tetY1770 |
| | | GAA CCA CCG GCA | | 1701 | | | | | |
| | | TGA TTA ATC CCA | | | | | | | |
| | | AAC CAA TCG CAT | | | | | | | |
| | | CAA GCG CGA CAA | | | | | | | |
| | | CAA TGA GTG C | | | | | | | |
| catI | chloramphenicol | TTT ACG GTC TTT | 70 | 550- | 73.1 | 41.1 | cam, | M62822 | 70-cat550 |
| | | AAA AAG GCC GTA | | 481 | | | Tn9, | | |
| | | ATA TCC AGC TGA | | | | | R100, | | |
| | | ACG GTC TGG TTA | | | | | cat, | | |
| | | TAG GTA CAT TGA | | | | | . . . | | |
| | | GCA ACT GAC T | | | | | | | |
| catII | chloramphenicol | AGC GGT AAT ATC | 70 | 300- | 75.6 | 45.7 | catII | X53796 | 70-cat |
| | | GAG TTT GGT GGT | | 231 | | | | | (2)300 |
| | | CAG GCT GAA TCC | | | | | | | |
| | | GCA TTT AAT CTG | | | | | | | |
| | | CTG ACG ATA AAG | | | | | | | |
| | | GGC AAA GTG T | | | | | | | |
| catIII | chloramphenicol | TTT GCT TGT TAA | 70 | 370- | 74.4 | 41.4 | catIII | X07848 | 70-cat |
| | | GCT AAA ACC ACA | | 301 | | | | | (3)370 |
| | | TGG TAA ACG ATG | | | | | | | |
| | | CCG ATA AAA CTC | | | | | | | |
| | | AAA ATG CTC ACG | | | | | | | |
| | | GCG AAC CCA A | | | | | | | |
| floR | florfenicol et | GAC AAA GGC CGG | 70 | 384- | 82.3 | 60 | floR, | AF252855 | 70-floR384 |
| | chloramphenicol | TGC AGT TGA AGA | | 315 | | | pp-flo | | |
| | | CCA AGC TGC TCC | | | | | | | |
| | | CAG AGA CGC AAT | | | | | | | |
| | | GAC GAA AGC CGT | | | | | | | |
| | | TGC GCC CGC A | | | | | | | |
| dhrf-I | trimethoprime | GGT TAA AGC ATC | 70 | 490- | 69.2 | 32.9 | dhfrl, | X00926 | 70-dhrf |
| | | TTT AAT TGA TGG | | 421 | | | (Tn7) | | (1)490 |
| | | AAA GAT CAA TAC | | | | | | | |
| | | GTT CTC ATT GTC | | | | | | | |
| | | AGA TGT AAA ACT | | | | | | | |
| | | TGA ACG TGT T | | | | | | | |
| dhrf-V | trimethoprime | GTA CAT GGC CTC | 70 | 1560- | 76.6 | 51.4 | dhrfV, | X12868 | 70-dhfr |
| | | TTC GAT CGA CGG | | 1491 | | | (dhfrb: | | (5)1560 |
| | | GAA TAC TAT TAC | | | | | 50%, | | |
| | | GTT GTC ATT ATC | | | | | dhrf | | |
| | | GGC CGT CCA GGC | | | | | XIV: | | |
| | | TGA GCG ATG A | | | | | 50%) | | |
| dhrf-VII | trimethoprime | GAA CAC CCA TAG | 70 | 753- | 64.2 | 72.4 | dhfr | X58425 | 70-dhfr |
| | | AGT CAA ATG TTT | | 684 | | | VII | | (7)753 |
| | | TCC TTC CAA CAA | | | | | (dhrfXV | | |
| | | GGA GCC ACT GAT | | | | | II: 95%, | | |
| | | TAT ATG TGA GCG | | | | | dhrfXV: | | |
| | | CTT TAA AGA G | | | | | 40%) | | |
| dhrf-IX | trimethoprime | AGC TTT GAA GTG | 70 | 830- | 72.5 | 40 | dhrflX | X57730 | 70-dhfr |
| | | TTT TAA ATC TTC | | 761 | | | | | (9)830 |
| | | TGG TTC ATG CCA | | | | | | | |
| | | CGG AAT CTG ATT | | | | | | | |
| | | TTC AAA TCC GAT | | | | | | | |
| | | ACC TCC TGT C | | | | | | | |
| dhrf-XIII | trimethoprime | TGG CGC GAG AGC | 70 | 929- | 82.1 | 58.6 | dhfr | X50802 | 70-dhfr |
| | | ACC ACT GTG TGG | | 860 | | | XIII | | (13)929 |
| | | CGG TTT GGT AAG | | | | | | | |
| | | GGC TTG CCT ATG | | | | | | | |
| | | GAC TCA AAT GTC | | | | | | | |
| | | TTG CGG CCC A | | | | | | | |
| dhrf-XV | trimethoprime | CTT CAG ATG ATT | 70 | 620- | 71.2 | 38.6 | dhfrXV | Z83311 | 70-dhfr |
| | | TAG CGC TTC ATC | | 551 | | | | | (15)620 |
| | | GAT AGA TGG AAA | | | | | | | |
| | | TAC CAA TAC ATT | | | | | | | |
| | | CTC ATC ACT GGA | | | | | | | |
| | | AGT GAA GCT T | | | | | | | |
| sulI | sulfonamide | AGC GCC GGC GGG | 70 | 960- | 82.5 | 62.9 | Tn21, | X12869 | 70-sul |
| | | GTC TAG CCG CCG | | 891 | | | Inte- | | (1)960 |
| | | GCT CTC ATC GAA | | | | | gron | | |
| | | GAA GGA GTC CTC | | | | | class | | |
| | | GGT GAG ATT CAG | | | | | 1, suII | | |
| | | AAT GCC GAA C | | | | | | | |
| sulII | sulfonamide | TAC GCG CCT GCG | 70 | 420- | 82.8 | 61.4 | RSF1010 | M36657 | 70-sul |
| | | CAA TGG CTG CGT | | 351 | | | suIII | | (2)420 |
| | | CTG GCG CCA GAT | | | | | | | |
| | | ACC GGC CTC CAT | | | | | | | |
| | | CGG AGA AAC TGT | | | | | | | |
| | | CCG AGG TTA T | | | | | | | |

| | | | | | | | | |
|---|---|---|---|---|---|---|---|---|
| Intergrase class II | TTG GAT GCC CGA | 70 | 1200- | 78.3 | 51.4 | Inte- | M33633 | 70-int |
| 3′ CS | GGC ATA GAC TGT | | 1131 | | | grase, | | (1)1200 |
| | ACC CCA AAA AAC | | | | | Int1 | | |
| | AGT CAT AAC AAG | | | | | | | |
| | CCA TGA AAA CCG | | | | | | | |
| | CCA CTG CGC C | | | | | | | |

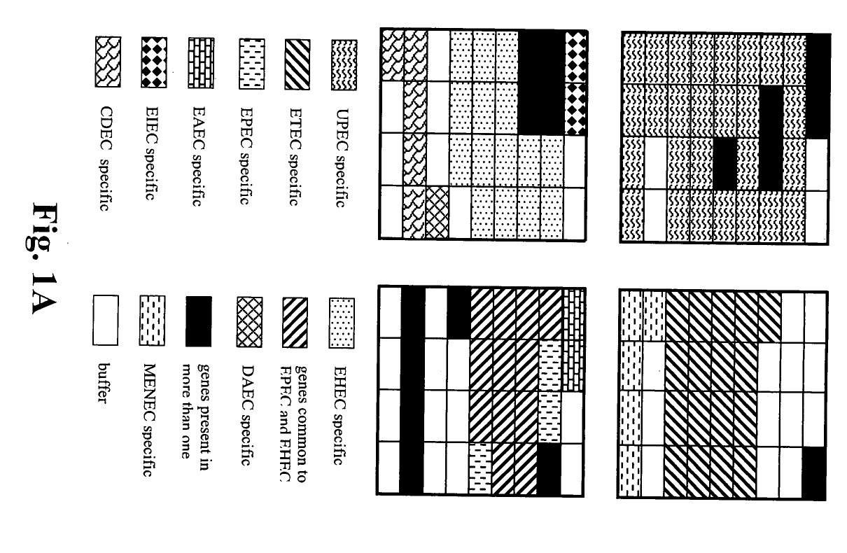

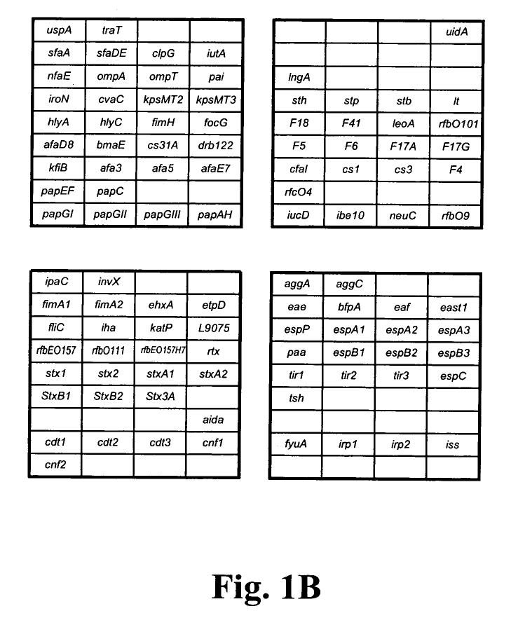

The DNA sequence of each gene was analyzed by BLAST analysis and ClustalW alignment followed by phylogenetic analysis. When the selected gene showed sequence divergence over 10% amongst different strains, new primers were designed to amplify the probe from each phylogenetic group as was the case for espA, espB and tir genes. The new primers were selected in conserved sequence areas flanking the area of divergence in order to ensure gene discrimination at the hybridization level. Phylogenetic analysis of the attaching and effacing locus (LEE) genes espA, espB and tir permitted us to distinguish three phylogenetic groups with regard to the sequence divergence cutoff value (<10%) chosen for this study. Attaching and effacing genes from strains EDL933, E2348/69 and RDEC-1 belonging to the different phylogenetic groups have been cloned and sequenced. Genomic DNA from strains EDL933 (EHEC), E2348/69 (Human EPEC) and RDEC-1 (rabbit EPEC) were used as templates to PCR amplify the different probes espA2-espB1-tir2, espA3-espB2-tir3 and espA1-espB3-tir1 respectively. The amplified probes were sequenced to confirm their identity and printed onto the pathotype microarray as shown in FIG. 1. For some virulence determinants, several genes of the cluster were targeted such as hly (hlyA, hlyC), pap (papAH, papEF, papC, papG), sfa (sfaDE, sfaa), agg (aggA, aggc). Utilization of several genes per cluster assisted in the confirmation of positive signals in addition to the assessment of cluster integrity. DNA probes detecting the genetic variants of Shiga-toxins (stx1, stx2, stxA1, sixA2, stxB1 and stxB2), cytolethal distending toxin (cdt1, cdt2 and cdt3), cytotoxic necrosing factor (cnf1, cnt2), and papG alleles (papGI, papGII and papGIII) were also included. In total, this gene sequence analysis resulted in the selection of 104 gene probes (Table 2).

Probe Amplification, Purification and Sequencing

E coli strains were grown overnight at 37° C. in Luria-Bertani medium. A 200 μl sample of the culture was centrifuged, the pellet was washed and resuspended in 200 μl of distilled water. The suspension was boiled 10 min and centrifuged. A 5 μl aliquot of the supernatant was used as a template for PCR amplification. PCR reactions were carried out in a total volume of 100 μl containing 50 pmol of each primer, 25 pmol of dNTP, 5 μl of template, 10 μl of 10×Taq buffer (500 mM KCl, 15 mM MgCl₂, 100 mM Tris-HCl, pH 9) and 2.5 U of Taq polymerase (Amersham-Pharmacia). PCR products were analyzed by electrophoresis on 1% agarose gels in TAE (40 mM Tris-acetate, 2 mM Na₂EDTA), then purified with the Qiaquick™ PCR Purification Kit (Qiagen, Mississauga, Ontario) and eluted in distilled water. Since the annealing temperature of the various PCR primers ranged from 40° to 65° C. and genomric DNA from 36 E. coli strains were used as template, all the PCR amplifications were done separately. A total of 103 virulence factor probes and two positive control probes, uidA and uspA, were amplified successfully as determined by amplicon size and DNA sequence. The purity of the amplified DNA was confirmed by agarose gel electrophoresis of 50-100 ng of each amplified fragment. The size of the PCR products ranged from 117 bp (east1) to 2121 bp (katP) with an average length of 500 bp for the majority of the DNA probes (Table 1). For quality control purposes all PCR fragments were partially sequenced for gene verification (Applied Biosystem 377 DNA sequencer using the dRhodamine Terminator Cycle Sequencing Ready™ reaction Kit).

Genomic DNA Extraction and Labeling

Cells, collected by centrifuging 5 ml of an overnight culture at 12,000 rpm, were washed with 4 ml of solution 1 (0.5 M NaCl, 0.01 M EDTA pH 8), resuspended in 1.2 ml of buffer 2 (solution 1 containing 1 mg/ml of lysozyme), then incubated at room temperature for 30 min. After proteinase K and SDS additions, a two hours incubation at 37° C. and a phenol-chloroform extraction, total DNA was precipitated by adding one volume of isopropanol. The harvested pellet was washed with one volume of 70% (v/v) ethanol, dried then resuspended in 100 μl of Tris-EDTA buffer. When desired, a volume of 5 ul of RNAse (10 mg/mL) was added to remove any trace of unwanted RNA in the suspension.

Before labeling, total DNA was reduced in size by restriction enzyme digestion (New England BioLabs, Mississauga, Ontario) and following digestion, the enzymes removed by phenol-chloroform extraction. Cy 3 dye was covalently attached to DNA using a commercial chemical labeling method (Mirus' Label IT™, PANVERA) with the extent of labeling depending primarily on the ratio of reagent to DNA and the reaction time. These parameters were varied to generate labeled DNA of different intensity. Two μg of the digested DNA were chemically labeled using 4 μl of Label IT™reagent, 3 μl of 10× Mirus™ labeling buffer A and distilled water in a 30 μl total volume. The reactions were carried out at 37° C. for 3 h. Labeled DNA was then separated from free dye by washing four times with water and centrifugation through Microcon™ YM-30 filters (Millipore, Bedford, USA). The amount of incorporated fluorescent cyanine dye was quantified by scanning the probe from 200 nm to 700 nm and subsequently inputting the data into the % incorporation calculator found at http://www. Dangloss.com/seidel/Protocols/Dercent inc.html. This method is based on the calculation of the ratio of μg of incorporated fluorescence: μg of labeled DNA. Alternatively, genomic E. coli DNA is fluorescently labeled with a simple random-priming protocol based on invitrogen's Bioprime DNA Labeling kit. The kit is used as a source of random octamers, reaction buffer, and high concentration klenow (40 U/pl). The dNTP mix provided in the kit, which contains biotin-labeled dCTP, is replaced by 1.2 mM dATP, 1.2 mM dGTP, 1.2 mM dTTP and 0.6 mM dCTP in 10 mM Tris pH 8.0 and 1 mM EDTA. In addition, 2 μl of Cy5-dCTP 1 mM from NEN were used to fluorescently label the DNA. The labeled samples are then purified on QIAquick™ columns according to the manufacturer's protocol after adding 2.5 μl 3 M NaOAcetate pH 5.2 to lower the pH of the solution. The microarrays are pre-hybridized for 1 hour at hybridization temperature with DIG buffer (Roche) and 10% (v/v) salmon sperm DNA (10 mg/ml), washed for 10 minutes in water and dried with gaseous; nitrogen 500 ng of labeled DNA, dried and resuspended in 6 μl of DIG buffer with salmon sperm DNA was used for the hybridization which is performed at 47° C. under a 11 mm×11 mm coverslip. Three stringency washes are performed after the hybridization: 1×SSC-0.2% (w/v) SDS at 42° C., 0.1×SSC-0.2% (w/v) SDS at 37° C. and 0.1×SSC at 37° C. The slide is dried with gaseous nitrogen and scanned.

Optimization of Microarray Detection Threshold Using a Prototype Microarray

A prototype chip was constructed and used to assess parameters, namely fragment length and extent of fluorescent labeling of the target (test) DNA, to optimize the spot detection threshold of the microarray. DNA amplicons from 34 E. coli virulence genes including the following EHEC virulence gene probes: espP, EHEC-hlyA, stx1, stx2, stxc, stxaII, paa and eae were generated by PCR amplification and printed in triplicate. The probe lengths ranged from 125 bp (east1) to 1280 bp (irp1). A HindIII/EcoRI digestion was used to generate large fragments (average size ˜6 Kb) and Sau3A/AluI digestion to produce smaller DNA fragments (average size ˜0.2 Kb) from E. coli O157:H7 strain STJ348 genomic DNA. The restricted DNAs were labeled and used as the target for hybridization with the prototype microarray. In the present experiments, the strongest hybridization signal was obtained by using larger fragments labeled at an optimal Cy3 rate in the range of 7.5 to 12.5. An estimate of the microarray's sensitivity was calculated by the following equation as described by De Boer and Beumer (De Boer, E., et al. (1999) Int J Food Microbiol. 50:119-130):
Sensitivity (%)=(number of true positive spots (p)/p+number of false negative spots)×100.
Construction of the E. coli Pathotype Microarray

Virulence factor probes were grouped by pathotype with the resulting array being composed of eight subarrays each corresponding to well characterized E. coli categories (FIG. 1). The enterohemorrhagic (EHEC) subarray included Shiga-toxin gene probes (stx1, stx2, stxA1, sbcA2, stxB1, stxB2 and stxB3), attaching and effacing genes, (espA, espb, tir, eae, and paa), EHEC specific pO157 plasmid genes (etpD, ehxA, L9075, katP, espP) and 0157 and 0111 somatic antigen genes (rtbE0157 and rfbO111). enteropathogenic E. coli (EPEC) was targeted by spotting LEE specific gene probes (eae, fir, espA, espB), espC and EPEC EAF plasmid probes (bfpA, eat). The enterotoxigenic subarray (ETEC) included probes for human heat-stable toxin (STaH), porcine heat-stable toxin (STaP), heat-stable toxin type II (STb), heat-labile toxin (LT), adhesion factors shared by human ETEC (CFAI, CS1, CS3, LngA) or by animal ETEC (F4, F5, F6, F18, F41). DNA probes for O101 specific somatic antigen (rtbO101) and ETEC toxin (leoA) were also included. To identify uropathogenic strains, the UPEC subarray was composed of 27 probes selected for detection of extraintestinal E. coli adhesins Pap (papGI, papGII, papGIII, papAH, papEF, papC), Sfa (sfaA, sfaDE), Drb (drb122), Afa (afa3, afa5, afaE7, afaD8), F1C (focG), nonfimbrial adhesin-1 (nfaE), M-agglutinin subunit (bmaE), CS31A (cIpG), toxins including hemolysins (hlyA and hlyC), cytotoxic necrosing factor (cnf1), and colicin V (cvaC), aembactin receptor (iutA), capsular specific genes kfiB (K5), kpsMTII (K1, K5, K12), KpsMTIII (K10, K54) in addition to the surface exclusion gene (traT) and uspA probes. The cell-detaching subarray (CDEC) contained toxin probes cnf1, cnf2, cdt1, cdt2 and cdt3. The genes iucD, neuC, ibe10, rfbO9 and rfO4 were designed to represent the meningitis-associated E. coli pathotype (MENEC). Enteroaggregative E. coli probes (EAEC) were derived from fimbrial specific genes aggA and aggC whereas enteroinvasive pathotype (EIEC) was targeted by invasin gene probes ipaC and invX. The AIDA (adhesin involved in diffuse adherence) probe was the unique marker for the diffusely adherent pathotype (DAEC).

Some virulence genes, such as fimA, fimH, irp1, irp2, iss, fyuA, ompA, east1, iha, fliC, tsh and ompT are shared by several E. coli pathotypes, and are thus indicative of subsets of pathotypes rather than specific to any one pathotype in particular. Finally a positive control, the uidA gene probe as well as a negative control composed of 50% (v/v) DMSO solution were added. An estimate of the specificity of the virulence microarray was calculated by the following equation (De Boer, E., et al. (1999) Int J Food Microbol. 50:119-130):
Specificity (%)=(number of true negative spots(n)/n+number of false positive spots)×100.
Printing and Processing of the Microarrays

Two μg of each DNA amplicon were lyophilized in a speed-vacuum and resuspended in filtered (0.22 μm) 50% (v/v) DMSO. The concentration of amplified products was adjusted to 200 ng/μl and 10 μl of each DNA amplicon were transferred to a 384-well microplate and stored at −20° C. until the printing step. DNA was then spotted onto CMT-GAPS™ slides (Corning Co., Corning, N.Y.) using a VIRTEK ChipWriter™ with Telechem SMP3™ microspotting pins. Each DNA probe was printed in triplicate on the microarray. After printing, the arrays were subjected to ultraviolet crosslinking at 1200 μJoules (U.V. Stratalinker™1800, STRATAGEN) followed by heating at 80° C. for four hours. Slides were then stored in the dark at room temperature until use.

Microarray Hybridization and Analysis

Microarrays were prehybridized at 42° C. for one hour under a 22×22 mm coverslip (SIGMA) in 20 μl of pre-warmed solution A (DIG Easy Hyb™ buffer, Roche, containing 10 μg of tRNA and 10 μg of denatured salmon sperm DNA). After the coverslip was removed by dipping the slide in 0.1×SSC (1×SSC contained 150 mM NaCl and 15 mM trisodium citrate, pH 7), the array was rinsed briefly in water and dried by centrifugation at room temperature in 50 ml conical tubes for five min at 800 rpm. Fluorescently-labeled DNA was chemically denatured as described by the manufacturer and added to 20 μl of a fresh solution of pre-warmed solution A. Hybridization was carried out overnight at 42° C. as recommended by the manufacturer. After hybridization, the coverslip was then removed in 0.1×SSC and the microarray washed three times in pre-warmed 0.1×SSC/0.1% (w/v) SDS solution and once in 0.1×SSC for 10 min at 50° C. After drying by centrifugation (800 rpm, five min, room temperature), the array was analyzed using a fluorescent scanner (Canberra-Packard, Mississauga, Ontario). The slides were scanned at a resolution of 5 μm at 85% laser power and the fluorescence quantified after background subtraction using QuantArray™ software (Canberra-Packard). All hybridization experiments were replicated between two to five times per genome.

### EXAMPLE 2

### Assessment of the Pathotype Microarray for Virulence Pattern Analysis

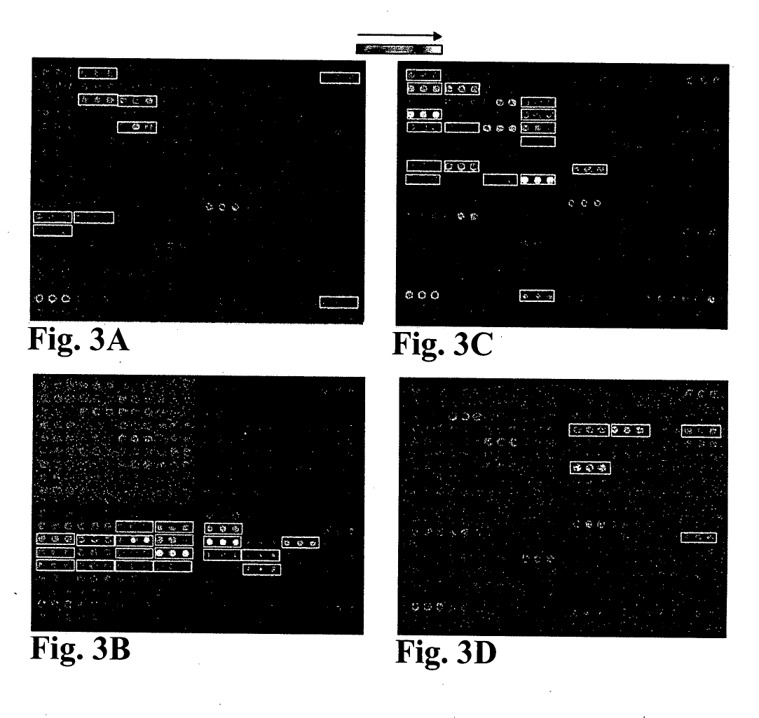

To identify known virulence genes and consequently, the pathotype of the E. coli strain being examined, genomic DNA from several previously characterized E. coli strains was labeled and hybridized to the pathotype microarray. The K12-derived E. coli strain DH5α was included as a nonpathogenic control. Interestingly, E. coli DH5α produced a fluorescent hybridization signal with the uidA, fimA₁, fimA₂, fimH, ompA, ompT, traT, fliC and iss probes (FIG. 3A). Genbank analysis of the sequenced K12 strain MG1655 genome revealed the presence of the first seven genes whereas the iss probe is 90% similar to ybcU, a gene encoding a bacteriophage lambda Bor protein homolog (sequence K12). Surprisingly, a false positive signal was obtained with the cdt1 and aggA gene probes. These genes are absent in the E. coli K12 genome and their sequences are not homologous to any K12 genes. Moreover, these genes were not positive with K12 or O157:H7 strain EDL933 in earlier generations of the virulence chip. The signal is the result of amplicon contamination in the final printing. Therefore, these two probes were not included in all subsequent hybridization analyses.

Since the genomic sequence of E coli O157:H7 strain EDL933 is available on GENBANK (NC_{—}002655), this strain represented a good choice to assess the detection threshold and hybridization specificity of the E. coli virulence factors on the microarray. After hybridizing the pathotype microarray with Cy3-labeled genomic DNA from E. coli O157:H7, the scanned image (FIG. 3B) showed fluorescent signals with the EHEC specific genes encoding Shiga-toxins, the attaching and effacing cluster present in EHEC and EPEC E. coli, the genes carried on the EHEC pO157 plasmid, antigen and flagellar specific genes as well as iha, an adhesin encoding gene (AF401752) found in both the EHEC and UPEC pathotypes. Therefore the EHEC pathotype of E. coli 0157:H7 was easily confirmed by a rapid visual scan of the virulence gene pattern (FIG. 1) of the scanned image.

The UPEC strain J96 (O4:K6) is a prototype E. coli strain from which various extraintestinal E. coli virulence factors have been cloned and characterized. This strain possesses two copies of the gene clusters encoding P (pap-encoded) and P-related (prs-encoded) fimbriae, produces FIC (focG), contains two hly gene clusters encoding hemolysin and produces cytotoxic necrosing factor type 1 (cnf1). E. coli strain J96 DNA was labeled and hybridized to the pathotype microarray. The scanned array resulted in a UPEC pathotype hybridization pattern (FIG. 3C). All of the UPEC virulence genes cited above were detected, as well as other uropathogenic specific genes. From a taxonomic perspective, the microarray also permitted the detection of the O4 antigen gene (rfcO4).

An enterotoxin-producing strain of E. coli isolated from a case of cholera-like diarrhea, E. coli strain H-10407, was used as a control strain to assess the ability of the microarray to identify the ETEC pathotype (FIG. 3D). Hybridization results showed the presence of a heat-stable enterotoxin Stah, antigenic surface-associated colonization factor cfaI, heat-labile enterotoxin LT, east1 toxin, and a weak signal was obtained with stap probe. The hybridization pattern correlated well with the virulence profile and pathotype group of this strain.

### EXAMPLE 3

### Determination of Virulence Patterns of Uncharacterized Clinical E. Coli Strains

To further validate the pathotype chip, virulence gene detection was assessed by hybridization with genomic DNA from five clinical E. coli strains isolated from human (H87-5406) and animal (Av01-4156, B004830, Ca01-E179, B99-4297) sources. Genomic DNAs from these strains were fragmented and Cy3-labeled and the microarray hybridization patterns obtained were compared with PCR amplification results.

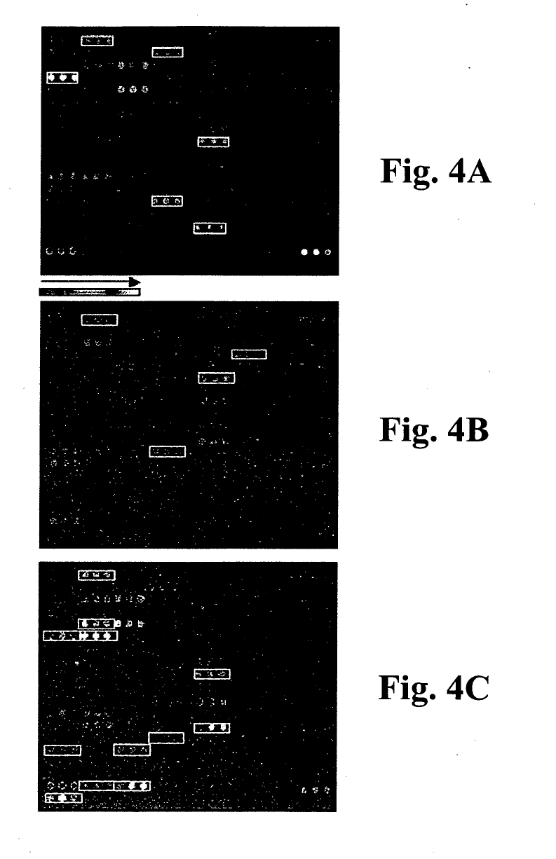

The virulence gene pattern obtained after microarray hybridization analysis with Cy3-labeled E. coli genomic DNA of avian-origin (Av01-456) showed the presence of the extra-intestinal E. coli virulence genes (iucD, iroN, traT, iut4) and genes present in our K12 strain (fimA1, fimA2, fimH, iss, ompA, and ompt) (FIG. 4A). The temperature-sensitive hemagglutinin gene (tsh) that was often located on the ColV virulence plasmid in avian-pathogenic E. coli (APEC) was also detected on the Av01-4156 virulence gene array. A strong hybridization signal was also obtained with the rtx probe derived from a gene located on the O157:H7 chromosome and encoding a putative RTX family exoprotein. The overall virulence factor detection pattern indicates that this strain is involved in extraintestinal infections.

When the pathotype microarray was hybridized with genomic DNA from strain B004830 isolated from bovine ileum, genes encoding ETEC fimbriae F5 and heat stable toxin StaP were detected (FIG. 4B) indicating that this strain belongs to animal ETEC pathotype. The hybridization pattern also showed the presence of traT, ompA, fimA1, fimA2, formH, fliC genes and the EHEC-associated gene etpD.

The virulence pattern obtained after microarray hybridization analysis with Cy3-labeled human-origin E. coli genomic DNA H87-5406 strain was very complex and did not fall within a single pathotype category. The hybridization pattern revealed the presence of espP, iss, rtx, fimA1, formA2, fimH, ompA, and ompT genes as well as Shiga-toxin gene, stx1, detected in the enterohemorragic pathotype (FIG. 4C). Moreover, virulence genes involved in extra-intestinal infections (cdt2, cdt3, afaD8, bmaE, iucD, iroN, traT and iutA) were also observed. Strain H87-5406 was also positive for the type 2 cytotoxic necrosing factor encoded by cnf2 gene.

The virulence patterns of two other isolates, the pulmonary isolated strain Ca01-E179 and the bovine strain B994297 (used elsewhere in this study) were clearly identified as UPEC pathotype and Shiga-toxin positive E. coli respectively. The presence of all the pathotype-specific virulence factors that were positively identified by the microarray data for the above animal and human isolates, was further confirmed by PCR amplification of each positive signal.

### EXAMPLE 4

### Discrimination Between Homologous Genes Belonging to Different Subclasses

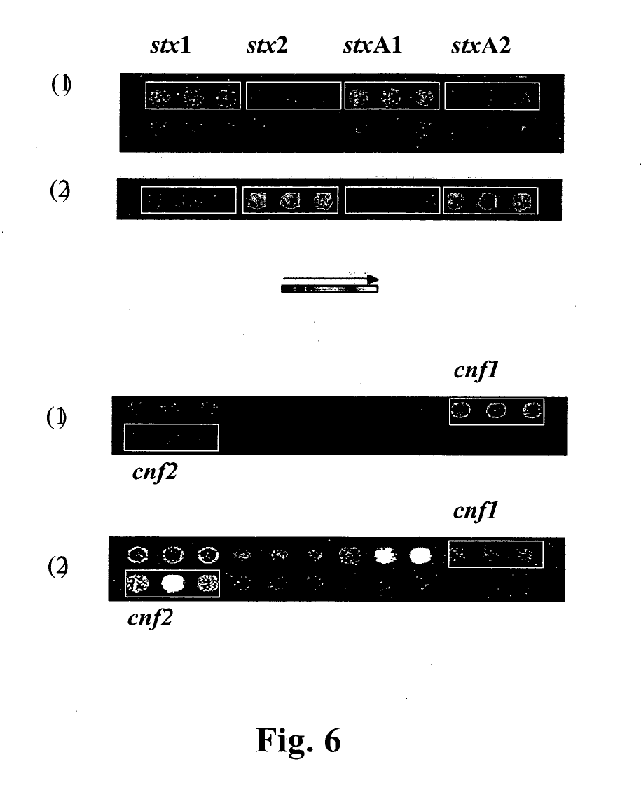

Given the importance of the stx gene family, amplicons sbcA1 and stxA2 specific for the A subunits of the stc1 and stx2 family (Table 5) were designed, in addition to using the published amplicons stx1 and stx2 (Table 2) which overlap the A and B subunits of the genes. Sequence similarity is of the order of 57% between the published stx1 and stx2 amplicons; similarity between the stxA1 and stxA2 amplicons designed herein is slightly higher, at 61%. As shown in FIG. 6A, the DNA probes used in this study for detection of stx1 and stx2 gene variants were successful in distinguishing stx1 from stx2, using either the previously published amplicons or the stxA subunit probes.

To further explore the potential of microarrays to distinguish gene variants within homologous gene families, primers used for cnf1 and cnf2 probe amplification were derived from studies on the detection of cnf variant genes by PCR amplification. The resulting amplicons have 85% sequence similarity. Hybridization results obtained with genomic DNA from cnf-positive strains H87-5406 and Ca01-E1799 (FIG. 6B) showed a clear distinction on the microarray between cnf1 and cnf2 gene variants, a significant result given the high degree of similarity and the size (over 1 kb) of the amplicons used.

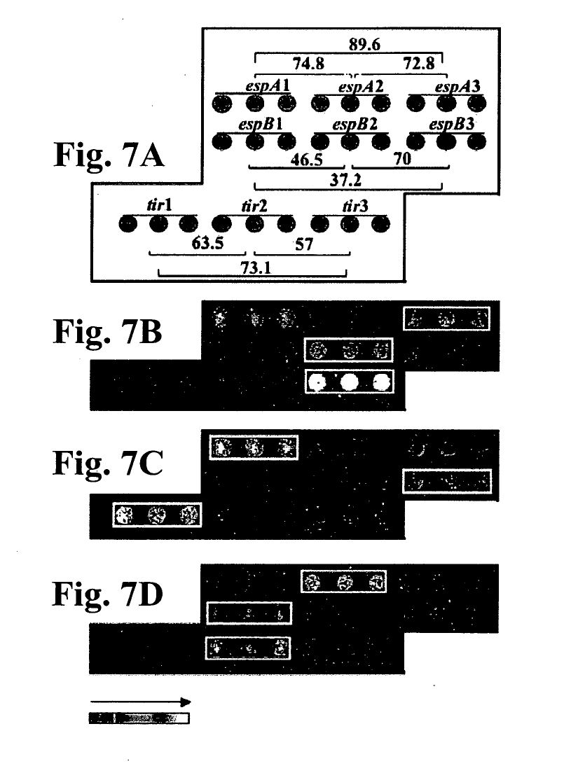

Since the DNA microarray showed initial promise in discriminating between the known gene variants of stx and cnf, a more defined group of genes were selected in order to test the ability of the pathotype microarray to differentiate between different phylogenetic groups of genes with a sequence divergence cutoff value of >10%. The DNA sequence similarity values of espA, espB and tir probes from the three different groups are summarized in FIG. 7A. The microarray was hybridized with labeled genomic DNA from EDL933 (EHEC) and E2348/69 (EPEC1) strains. Labeled DNA from another strain P86-1390 belonging to the same phylogenetic group as RDEC-1 was used to validate the hybridization specificity of the arrayed virulence genes. Hybridizations with the pathotype microarray were performed at 42° C. and 50° C. and, as shown in FIGS. 7B, C and D, the labeled DNA hybridized as expected to probes specific for each phylogenetic group. Genomic DNA from strain P86-1390 hybridized with espA1, espB3, tir1 probes, indicating that this strain belongs to the same group as RDEC-1, which correlates well with the phylogenetic analysis. A strong cross-hybridization signal was obtained between the espA1 and espA3 probes due to their high DNA-similarity score (89.6%). These hybridization patterns were obtained at 42° C. as well as at 50° C. indicating that DNA sequence divergences of 25% can be resolved under standard hybridization conditions. These results demonstrated that the pathotype microarray can be a useful tool for strain genotyping.

### EXAMPLE 5

### Antibiotic Resistance Assay on on Enterotoxigenic Escherichia coli

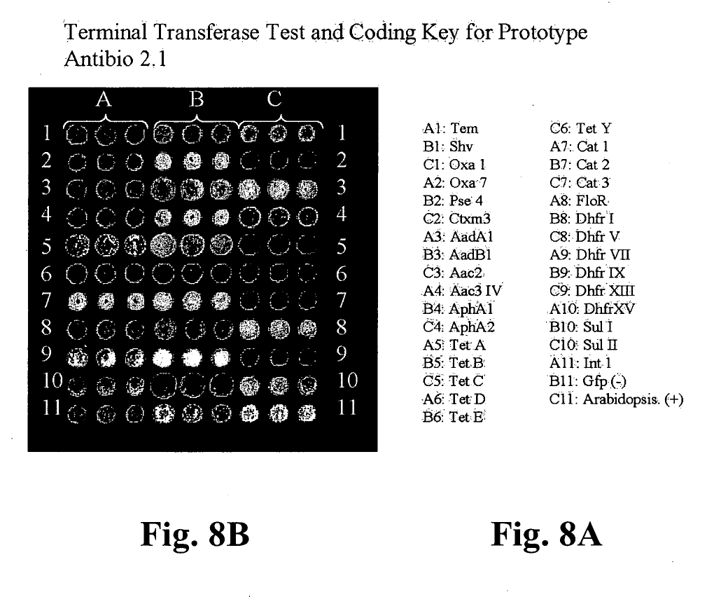

A prototype of microarray for testing antibiotic resistance has been constructed. FIG. 8 shows the coding key (8B) for the antimicrobial resistance gene prototype, together with a quality control test (8A) that shows that the probes for each gene were successfully immobilized on the DNA microarray.

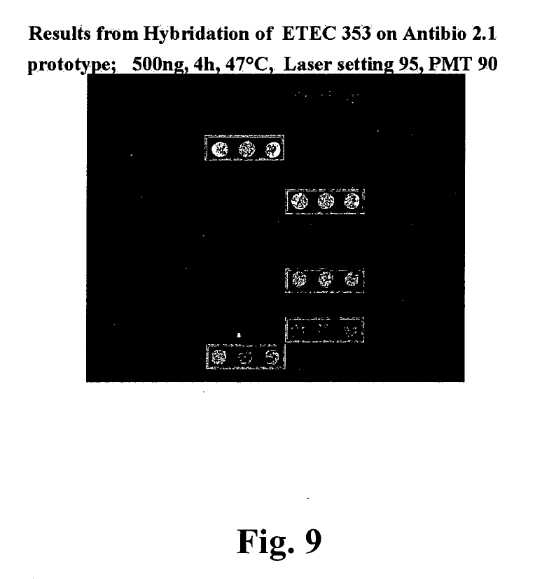

FIG. 9 shows results obtained with enterotoxigenic Escherichia coli (ETEC) strain 353 (from J. M. Fairbrother's collection). The fluorescent spots dearly indicate the presence of antimicrobial resistance genes corresponding to the known antimicrobial resistance phenotype of this isolate. The validity of these results has been confirmed independently by PCR and membrane hybridization.

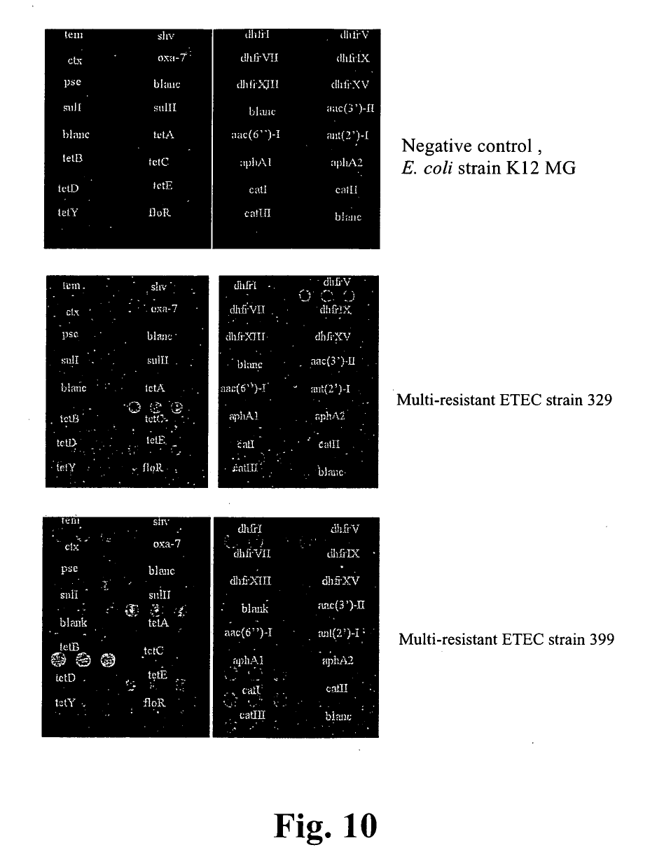

Other results in the form of a comparison between two multiresistant Escherichia coli enterotoxigenic strains (ETEC 329 and ETEC 399) are shown in FIG. 10, compared to a negative control E. coli which does not have antibiotic resistance genes. The spots visible for strains 329 and 399 clearly indicate the presence of several antibiotic resistance genes. The faint spots for the negative control can be clearly distinguished from the positive signal.

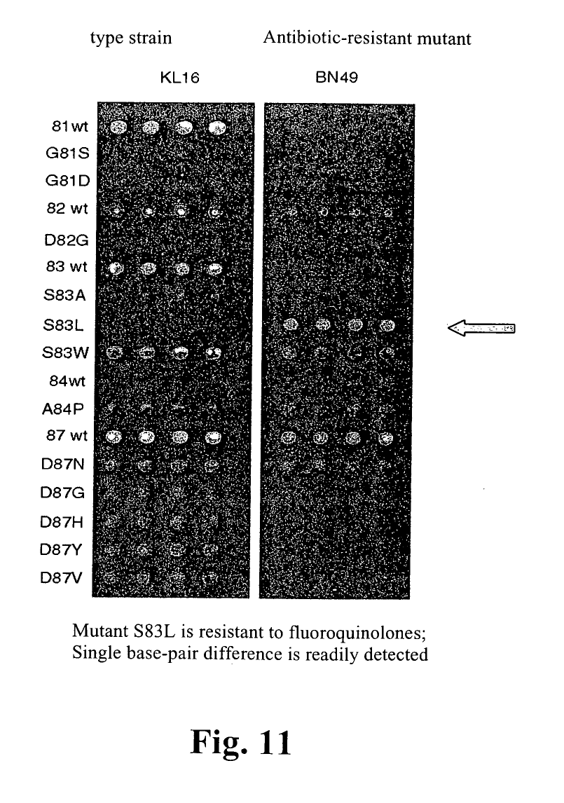

The present invention also allow to discriminate a single base pair mutation. FIG. 11 shows that careful application of the hybridization strategy described herein can distinguish the single base pair mutant involved in mutation S83L, involved in fluoroquinolone resistance in E. coli. The capacity to identify such subtle mutations is an important aspect of the invention.

In accordance with the present invention, there is provided together several known methods optimized to achieve the various steps described above. The key elements are i) the use of synthetic oligonucleotides as DNA probes (see Table 7 below for examples)—these are superior to generally used PCR amplicons in terms of ease of manufacture and purification, but require optimized DNA labeling and hybridization conditions in order to generate sufficient signal. The optimized DNA labeling procedures are described in Bekal et al. (Bekal, S., et al., Journal of Clinical Microbiology, 2003. 41 (5): p. 2113-2125), the disclosure of which is incorporated herein by reference; ii) the use of a bias-free, combined DNA amplification and labeling method to save time, reduce costs and, greatly improve sample processing and robustness of the procedure. Amplification is based upon commercial kits, which is generally known in the art; and iii) the use of shortened hybridization time under carefully controlled conditions to save time. Hybridization time has been shortened from overnight (18 h) to four hours, with partial results available after one hour, in one embodiment of the invention.

The studies described herein entailed designing a DNA microarray containing 103 gene probes distributed into eight subarrays corresponding to various E. coli pathotypes. To evaluate the microarray regarding the specificity of the amplified virulence factor gene fragments, genomic DNAs from different E. coli strains were labeled and hybridized to the virulence factor microarray. To this end, applicants developed a simple protocol for probe and target preparation, labeling and hybridization. The use of PCR amplification for probe generation, and fragmented genomic DNA as labeled target allowed the detection of all known virulence factors within characterized E. coli strains. Direct chemical labeling of genomic DNA with a single fluorescent dye (Cy3) facilitated the work.

Since the fluorescent assay used herein was based on direct detection (single Cy dye) rather than differential hybridization (multiple dyes), optimization of the signal detection threshold was performed. It was determined that the signal intensity, apart from DNA homology and DNA labeling efficiency, depended on (i) immobilized amplicon size (ii) gene copy number in target genomic DNA and (iii) size of the labeled target DNA. Within the large range of probe sizes (117 bp and 2121 bp) tested, hybridization signal intensity could be affected by probe length when using homologous DNA. Quality control analysis of the printed microarray using terminal transferase showed heterogeneity in the spotted amplicons. Using two strains with known genomes (K12 and EDL933), the level of accuracy (sensitivity and specificity) of the current virulence/antibiotic resistance chip as outlined in the Examples herein can be estimated. The average sensitivity or accuracy in discriminating among the different virulence or antibiotic resistance genes approached 97%.

Gene location is another factor to consider when designing gene detection microarrays. After hybridization with genomic DNA from E. coli O157:H7 strain EDL933, it was found strong hybridization signals to etpD, ehxA, L7590, katP and espP. Since these genes are located on the pO157 plasmid (Accession number AF074613), the stronger signal can be attributed to a higher copy number or gene dose. Moreover, many virulence genes are located on mobile elements like plasmids, phages, or transposons and are encoded by foreign DNA acquired via horizontal gene transfer and inserted in the genome. These pathogenicity islands (PAIs) are highly unstable and are constantly shuttled between strains. However, in addition to their total horizontal transfer or deletion, several studies suggested that PAIs are subject to continuous modifications in their virulence factor composition. In earlier work, the detection of a single PAI gene reflected the presumed presence of all the additional virulence genes encoded by the PAI but due to the potential for genetic rearrangements described above, this assumption is risky. Microarray technology represents an excellent tool to circumvent this PAI plasticity and identify genetic rearrangements by gene deletion or insertion on PAI clusters.

Recent investigations of E. coli virulence have revealed new information regarding the prevalence of virulence genes within a specific E. coli pathotype. For example the cytolethal-distending factor (cdt) was first described as virulence factor associated with EPEC E. coli and other diarrhea-associated pathotypes. Later, this gene was detected in strains involved in extraintestinal infections in humans and dogs. More recently, cdt and the urinary tract infection-associated gene (omp T) have been found to be as or more prevalent than traditional neonatal bacterial meningitis NBM-associated traits, such as ibeA, sfaS, and K1 capsule. The usefulness of the virulence microarray concept for exploring the global virulence pattern of strains and the potential detection of unexpected virulence genes was revealed by total genomic hybridizations with uncharacterized clinical strains. The rtx probe (encoding a putative RTX family exoprotein, accession number AE005229) located on the O157:H7 chromosome was amplified using genomic DNA from strain EDL933. Blast analysis did not reveal significant similarities with any available sequences. Analysis of the hybridization patterns of the extraintestinal strain Av014156 and strain H87-5406 revealed a strong signal with the rtx probe indicating the presence of a gene homologous to the rtx probe (FIG. 4). This gene was successfully amplified in both strains using the fix-specific primers. To the inventors' knowledge, this is the first report of the presence of this gene in non-0157 strains.

The potential for possessing different combinations or sets of virulence genes within a given E. coli strain could lead to the emergence of new pathotypes. Consistent with this hypothesis, it was found that in the clinical strain H87-5406, a combination of virulence factors from different pathotypes was observed. Moreover, microarray hybridization permitted detection of the Shiga-toxin gene stx1 associated with EHEC strains in addition to virulence genes involved in extra-intestinal infections (cdt2, cdt3, afaD8, bmaE, iucD, iroN, traT, iutA). Starcic et al. (Starcic, M., et al. (2002) Vet Microbiol. 85:361-77) recently reported a case of a “bifunctional” E. coli strain isolated from dogs with diarrhea. When analyzed, only a few strains were positive for heat stable toxin (ST) and none of them produced diarrhea-associated fimbriae K88 or K99 in contrast with previous studies. However, most of these strains were positive for cytonecrosing toxin (cnf1) as well as P-fimbriae and hemolysin (hly) that are involved in extra-intestinal infections in humans and animals. It was thus concluded that hemolytic E. coli isolated from dogs with diarrhea have characteristics of both uropathogenic and necrotoxigenic strains.

Another example illustrating the ability of the virulence microarray to provide a more thorough analysis of virulence genes and consequently the detection of potentially new pathotypes is further supported by the present study in which the ETEC pathotype of the bovine clinical strain B00-4830 was confirmed. In addition to the presence of the ETEC-associated virulence genes encoding StaP and F5 revealed in the hybridization pattern, the etpD gene, described by Schmidt et al. (Schmidt, H., et al. (1997) FEMS Microbiol Lett. 148:265-72) as an EHEC type 11 secretion pathway, was unexpectedly found to be present. In their study, Schmidt et al. (supra), reported that the etp gene cluster was detected in all 30 of the EHEC strains tested by hybridization (using the 11.9 Kb etp cluster from EDL933 as a probe) and by PCR using etpD-specific primers. However, none of the other E. coli pathotypes tested (EPEC, EAEC, EIEC, and ETEC) were positive for the etp gene cluster. As our results are contrary to this study, we assayed for the presence of the etpD gene in strain B00-4830 by PCR using the reverse primer described by Schmidt et al (supra) and a forward one designed in our study. Amplification of the expected 509 bp fragment was consistent with the microarray results confirming that etpD gene can be found in ETEC strains.

Another unexpected finding of the study described herein was the prevalence of fimH and ompT genes that have been epidemiologically associated with extraintestinal infections. BLAST analysis of ompT and fimH genes indicated the presence of both genes in E. coli K12 strain MG1655 and in enterohemorrhagic E. coli O157:H7 strain EDL933 and strain RIMD 0509952. In addition, the hybridization results herein revealed the presence of the formH gene in all strains tested in this study, including non-pathogenic E. coli, EPEC, ETEC and UPEC strains. The ompT gene was less prevalent but present in the Shiga-toxin producing strain H87-5406. It was also found in another Shiga-toxin producing strain B99-4297 as well as in the EPEC strains P86-1390 and E2348/69. The use of these genes as indicators of the UPEC pathotype should be reconsidered.

The studies described herein thus demonstrate that DNA microarray technology can be a valuable tool for pathotype and antibiotic resistance identification and assessing the virulence potential and the antibiotic resistance of E. coli strains including the emergence of new pathotypes or new resistances. The DNA chip design described herein should facilitate epidemiological and phylogenetic studies since the prevalence of each virulence and antibiotic resistance gene can be determined for different and strains and the phylogenefic associations elucidated between virulence pattern and serotypes of a given strain. In addition, unlike traditional hybridization formats, microchip technology is compatible with the increasing number of newly recognized virulence and resistance genes since thousands of individual probes can be immobilized on one chip.

The DNA labeling methodology, hybridization and pathotype/antibiotic resistance assessment described herein is both rapid and sensitive. The applications of such microarrays extend broadly from the medical field to drinking water, food quality control and environmental research, and can easily be expanded to virulence and antibiotic resistance gene detection in a variety of microorganisms.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. An array comprising:
(a) a substrate; and f
(b) a plurality of nucleic acid probes, each of said probes being bound to said substrate at a discrete location;
said plurality of probes comprising at least one probe for a pathotype of a species of a microorganism and at least one other probe for an antibiotic resistance gene of said species.

2. The array of claim 1, comprising at least two probes for a pathotype, wherein said at least two probes are not identical.

3. The array of claim 1, comprising at least two probes for an antibiotic resistance gene, wherein said at least two probes are not identical.

4. The array of claim 2 wherein said array comprises a subarray, wherein said subarray comprises said at least two probes at adjacent discrete locations on said substrate.

5. The array of claim 1 wherein at least one of said plurality of probes is for a virulence gene or a fragment thereof or a sequence substantially identical thereto, wherein said virulence gene is associated with pathogenicity of said microorganism.

6. The array of claim 1, wherein said microorganism is a bacterium.

7. The array of claim 6, wherein said bacterium is of the Enterobactefiaceae family.

8. The array of claim 7, wherein said bacterium is E. coli.

9. The array of claim 1, wherein said pathotype is selected from the group consisting of:
a) enterotoxigenic E. coli (ETEC);
b) enteropathogenic E. coli (EPEC);
c) enterohemorrhagic E. coli (EHEC);
d) enteroaggregative E. coli (EAEC);
e) enteroinvasive E. coli (EIEC);
f) uropathogenic E. coli (UPEC);
g) E. coli strains involved in neonatal meningitis (MENEC);
h) E. coli strains involved in septicemia (SEPEC);
i) cell-detaching E. coli (CDEC); and
j) diffusely adherent E. coli (DAEC).

10. The array of claim 1, wherein said antibiotic resistance gene is selected from the group consisting of aac(3)-IV, aac(3)-IIa, aac(3′-II, aac(6), aac(6′)-aph(2′), aac(6′)-Ii, ant(2″-Ia, ant(2′)-IIb, ant(2′)-laant(3″)-Ia, ant(3′)-Ia, ant(4′), ant(9)-Ia, aph(2″)-Id, aph(3′)-IIIa, aph(3′)-Ia, aph(3′)-Ia, aph(3′)-Ia, aph(3)-IIa, bla_{CTX-M-3}, ba_{OXA-1}, bla_{OXA-7}, bla_{PSE-4}, bla_{SHV}, bla_{TEM}, blaZ, catI, catII, catIII, Class 1 integron, dhfrO, dhfrIX, dhfrV, dhfrVII, dhfrXIII, dhfrXV, ermA, ermB, ermC, ermTR, floR, linA, mecA, mefA, mrsB, msrA, mupR, sat4, sulI, sulII, tet(A), tet(B), tet(C), tet(D), tet(E), tet(K), tet(L), tef(M), tet(O), tet(O), tet(S), tet(Y), tet(A)P, vanA, vanB, vanC, vanC3, vanD, vanE, vatA, vatC, vatD, vatE vga, vgb, and vgbB.

11. The array of claim 9, wherein said pathotype is selected from the group consisting of enteroaggregative E. coli (EAEC), enteroinvasive E. coli (EIEC), E. coli strains involved in neonatal meningitis (MENEC), E coli strains involved in septicemia (SEPEC), cell-detaching E. coli (CDEC), and diffusely adherent E. coli (bAEC).

12. The array of claim 5, wherein said virulence gene encodes a polypeptide of a class of proteins selected from the group consisting of toxins, adhesion factors, secretory system proteins, capsule antigens, somatic antigens, flagellar antigens, invasins, autotransporter proteins, and aerobactin system proteins.

13. The array of claim 5, wherein said virulence gene is selected from the group consisting of afaBC3, afaE5, afaE7, afaD8, aggA, aggC, aida, bfpA, bmaE, cdt1, cdt2, cdt3, cfaI, clpG, cnf1, cnf2, cs1, cs3, cs31a, cvaC, derb122, eae, eaf, east1, ehxA, espA group I, espA group II, espA group III, espB group I, espB group II, espB group III, espC, espP, etpD, F17A, F17G, F18, F4, F41, F5, F6, fimA group I, fimA group II, fimH, fliC, focG, fyuA, hlyA, hlyC, ibe10, iha, invX, ipaC, iroN, irp1, irp2, iss, iucD, iutA, katP, kfiB, kpsMTII, kpsMTIII, 17095, leoA, IngA, it, neuC, nfaE, ompA, ompT, paa, papAH, papC, papEF, papG group I, papG group II, papG group III, pai, rfb O9, rfb O101, rfb O111, rfbE O157, rfbE O157H7, rfc O4, rtx, sfaDE, sfaA, stah, stap, stb, stx1, stx2, stxA I, stxA II, stxB I, stxB II, stxB III, tir group I, tir group II, tir group III, traT, and tsh.

14. The array of claim 1 wherein said probe comprises at least one nucleic acid sequence selected from the group consisting of SEQ ID NO:1 to SEQ ID NO:104, or a fragment thereof, or a sequence substantially identical thereto.

15. The arrays of claim 1, wherein said probe is made of oligonucleotides to provide fine resolution of small genetic differences that may be of interest in pathogenicity and antibiotic resistance determination.

16. The array of claim 1, wherein said probe comprises at least one nucleic acid sequence from the group shown in Table 7, or a fragment thereof, or a sequence substantially identical thereto.

17. A method of detecting the presence of a microorganism in a sample, said method comprising:
(a) contacting the array of claim 1 with a sample nucleic acid of said sample; and
(b) detecting association of said sample nucleic acid to at least one of said plurality of nucleic acid probes on said array;
wherein association of said sample nucleic acid with at least one of said plurality of nucleic acid probes is indicative that said sample comprises a microorganism having a virulence gene and an antibiotic resistance gene from which the nucleic acid sequence of said probes is derived.

18. The method of claim 17, wherein said method further comprises extracting said sample nucleic acid from said sample prior to contacting it with said array.

19. The method of claim 17, wherein said sample nucleic acid is not amplified by PCR prior to contacting it with said array.

20. The method of claim 17, wherein said method further comprises digesting said sample nucleic acid with a restriction endonuclease to produce fragments of said sample nucleic acid.

21. The method of claim 20, wherein said fragments are of an average size of about 0.2 Kb to about 12 Kb.

22. The method of claim 17, wherein said sample is selected from the group consisting of environmental sample, biological sample and food.

23. The method of claim 22 wherein said environmental sample is selected from the group consisting of water, air and soil.

24. The method of claim 22 wherein said biological sample is selected from the group consisting of blood, urine, amniotic fluid, feces, tissues, cells, cell cultures and biological secretions, excretions and discharge.

25. The method of claim 13, wherein said sample is a tissue, body fluid, secretion or excretion from a subject.

26. A method for simultaneously determining a pathotype of a species of said microorganism and antibiotic resistance of said microorganism in a sample, said method comprising:
(a) contacting the array of claim 1 with a sample nucleic acid of said sample; and
(b) detecting association of said sample nucleic acid to at least one of said plurality of nucleic acid probes on said array;
wherein association of said sample nucleic acid with at least one of said plurality of nucleic acid probes is indicative that said microorganism is of said pathotype and has an antibiotic resistance gene from which the nucleic acid sequence of said probes is derived.

27. A method for diagnosing an infection by a microorganism in a subject, said method comprising:
(a) contacting the array of claim 1 with a sample nucleic acid of said subject; and
(b) detecting association of said sample nucleic acid to at least one of said plurality of nucleic acid probes on said array;
wherein association of said sample nucleic acid with at least one of said plurality of nucleic acid probes is indicative that said subject is infected by a microorganism having a virulence gene and an antibiotic resistance gene from which the nucleic acid sequence of said probes is derived.

28. The method of claim 27, wherein said subject is a mammal.

29. The method of claim 25, wherein said subject is a human.

30. A commercial package comprising the array of claim 1 together with instructions for
(a) detecting the presence of a microorganism in a sample;
(b) determining the pathotype of a microorganism in a sample;
(c) determining antibiotic resistance of a microorganism in a sample;
(d) diagnosing an infection by a microorganism in a subject;
(e) diagnosing a condition related to infection by a microorganism, in a subject; or
(f) any combination of (a) to (e).

31. A method of producing an array for simultaneously detecting virulence and antibiotic resistance of a microorganism in a sample, said method comprising:
a) providing a plurality of nucleic acid probes, said plurality of probes comprising at least one probe for a pathotype of a species of said microorganism and at least one probe for an antibiotic resistance gene of said species; and
b) applying each probe of said plurality of probes to a different discrete location of a substrate.

32. A method of producing an array for simultaneously detecting virulence and antibiotic resistance of a microorganism in a sample, said method comprising:
a) selecting a plurality of nucleic acid probes, said plurality of probes comprising at least one probe for a pathotype of a species of said microorganism and at least one probe for an antibiotic resistance gene of said species; and
b) synthesizing each of said plurality of probes at a different discrete location of a substrate.
